(19) United States
(12) Patent Application Publication
(10) Pub. No.: US 2023/0293686 A1
(43) Pub. Date: Sep. 21, 2023

(54) COMPOSITIONS AND METHODS FOR MODULATION OF SIRPALPHA-MEDIATED SIGNALING

(71) Applicant: The Board of Trustees of the Leland Stanford Junior University, Stanford, CA (US)

(72) Inventors: Kenan Christopher GARCIA, Menlo Park, CA (US); Ricardo A. FERNANDES, Oxford (GB); Junming REN, Palo Alto, CA (US)

(21) Appl. No.: 18/006,119

(22) PCT Filed: Jul. 22, 2021
(86) PCT No.: PCT/US2021/042767

§ 371 Date: Jan. 19, 2023

Related U.S. Application Data
(60) Provisional application No. 63/056,156, filed on Jul. 24, 2020.

(51) Int. Cl. A61K 39/00; C07K 14/705; C07K 16/28; A61P 35/00; C12N 5/0784

(52) U.S. Cl. CPC A61K 39/4615; A61K 39/0011; A61P 35/00; C07K 14/70503; C07K 16/2803; C07K 16/289; C12N 5/0639; A61K 2039/5154; C07K 2317/622; C07K 2319/00; C12N 2501/599; C12N 2501/998

(57) ABSTRACT

The present disclosure relates generally to compositions and methods for modulating cell surface receptor signaling by specifically recruiting membrane phosphatases, in cis, to a spatial proximity of a signal regulatory protein α (SIRPα) molecule. More particularly, the disclosure provides novel multivalent protein-binding molecules that specifically bind SIRPα and antagonize the SIRPα-mediated signaling through recruitment of a phosphatase activity to dephosphorylate the intracellular domain of SIRPα. Also provided are compositions and methods useful for producing such molecules, methods for promoting maturation dendritic cells and for production of vaccine, as well as methods for the prevention and/or treatment of health conditions associated with the inhibition of signal transduction mediated by SIRPα and/or CD47.

### STATEMENT REGARDING FEDERALLY SPONSORED R&D

This invention was made with Government support under contract CA177684 awarded by the National Institutes of Health. The Government has certain rights in the invention.

### CROSS-REFERENCE TO RELATED APPLICATION

The present application claims priority to U.S. Provisional Pat. Application Serial No. 63/056,156, filed on Jul. 24, 2020, the disclosure of which is incorporated by reference herein in its entirety, including any drawings.

### INCORPORATION OF THE SEQUENCE LISTING

This application contains a Sequence Listing, which is hereby incorporated herein by reference in its entirety. The accompanying Sequence Listing text file, named “Sequence Listing_078430-523001WO_ST25.txt,” was created on Jul. 19, 2021 and is 40 KB.

### FIELD

The present disclosure relates generally to the field of immuno-therapeutics, and particularly relates to multivalent protein-binding molecules designed to specifically bind a signal-regulatory protein α (SIRPα) molecule and antagonize SIRPα-mediated signaling through recruitment of a phosphatase activity. The disclosure also provides compositions and methods useful for producing such multivalent polypeptides, methods for promoting maturation dendritic cells and for production of vaccine. Also provided are compositions and methods useful for the prevention and treatment of health conditions associated with the inhibition of signal transduction mediated by SIRPα and/or CD47.

### BACKGROUND

Signal-regulatory protein α (SIRPα) is an innate immune checkpoint receptor expressed primarily on myeloid cells, including monocytes, macrophages, dendritic cells (DCs), and neutrophils. SIRPα suppresses innate immunity upon interaction with its ligand, CD47. CD47 is broadly expressed on normal tissues and is up-regulated by virtually all human tumors in order to escape macrophage recognition and programmed cell removal. It has been reported that this SIRPα/CD47 interaction negatively controls effector function of innate immune cells such as host cell phagocytosis. In particular, inhibitory signals delivered by CD47 through SIRPα diminishes FcyR-dependent antibody effector functions, including macrophage and neutrophil-mediated antibody-dependent cellular phagocytosis (ADCP) and cytotoxicity (ADCC), limiting the induction of antibody-dependent innate immunity and promoting resistance to antitumor antibody therapy.

In recent year, targeting the CD47-SIRPα pathway represents a novel therapeutic approach to enhance anti-cancer immunity by promoting both innate and adaptive immune responses. Unlike CD47, which is expressed ubiquitously, SIRPα expression is mainly restricted to myeloid cells. Therefore, compared to CD47-targeted therapies, targeting SIRPα may result in differential safety and efficacy profiles, potentially enabling lower effective doses and improved pharmacokinetics and pharmacodynamics.

Currently, for antagonism of SIRPα, the most prevalent strategy is through blockade of ligand binding between the extracellular domains (ECDs) of SIRPα through the use of, for example, antagonistic antibodies directed to an ECD of SIRPα. In this scenario, the blocking molecules (e.g., the antagonist antibodies) function by competing with the natural ligand for binding to the ECD of SIRPα. However, the development of effective SIRPα antagonistic antibodies has been reported to be challenging and restricted by polymorphisms within the CD47-binding domain of SIRPα, which necessitates pan-allele reactive anti-SIRPα antibodies for therapeutic intervention in diverse patient populations. In addition, these blocking antibodies have been reported to be ineffective in many patients, and not capable of eliminating the basal intracellular signaling activity of SIRPα (also referred to as resting intracellular signaling activity) that signals through phosphorylation mechanisms. This failure to eliminate basal signaling activity frequently limits the effectiveness of ECD ligand blocking strategies. Thus, new methods are needed to directly reduce or eliminate the intracellular signaling of SIRPα by alternative mechanisms other than ECD ligand blocking mechanism which would reduce or eliminate both resting and ligand-activated signaling.

Accordingly, there remains a need for alternative approaches other than direct SIRPα-ligand blockade by antibodies or other agents, to complement existing therapeutic standards of care for immunotherapy of cancer and other immune diseases.

### SUMMARY

The present disclosure relates generally to the immuno-therapeutics, such as multivalent polypeptides, multivalent antibodies, and pharmaceutical compositions comprising the same for use in treating various health conditions, such as those associated with the inhibition of cell signaling mediated by a cell surface receptor of interest. In particular, as described in greater detail below, some embodiments of the disclosure provide compositions and methods for modulating cell signaling mediated the SIRPα/CD47 pathway by, for example, specifically recruiting membrane phosphatases to a spatial proximity of the SIRPα through, for example, direct ligation using a multivalent protein-binding agent. More particularly, the disclosure provides novel multivalent protein-binding molecules that specifically bind to SIRPα and thereby completely or partially antagonizing the SIRPα signaling through recruitment of a phosphatase activity. This approach, termed “Receptor Inhibition by Phosphatase Recruitment” (RIPR), was described previously in, for example, WO2019/222547A1. In some particular embodiments, the multivalent protein-binding molecules of the disclosure are multivalent polypeptides. In some embodiments, the multivalent polypeptides are multivalent antibodies. The disclosure also provides compositions and methods useful for producing such multivalent polypeptides, methods for promoting maturation dendritic cells and for production of vaccine. Also provided are compositions and methods for the prevention and/or treatment of health conditions associated with the inhibition of signal transduction mediated by the SIRPα/CD47 pathway.

In one aspect, provided herein are multivalent polypeptides which include a first amino acid sequence including (a) a first polypeptide module capable of binding to a signal regulatory protein α (SIRPα); and (b) a second amino acid sequence comprising a second polypeptide module capable of binding to one or more receptor protein-tyrosine phosphatases (RPTPs) of R1/R6 subfamily.

Non-limiting exemplary embodiments of the multivalent polypeptide of the disclosure can include one or more of the following features. In some embodiments, the one or more RPTPs includes CD45 or a functional variant thereof. In some embodiments, at least one of the first and second polypeptide modules includes an amino acid sequence for a protein-binding ligand or an antigen-binding moiety. In some embodiments, the antigen-binding moiety is selected from the group consisting of a single-chain variable fragment (scFv), an antigen-binding fragment (Fab), a nanobody, a V_{H} domain, a V_{L} domain, a single domain antibody (dAb), a V_{NAR} domain, and a V_{H}H domain, a diabody, or a functional fragment of any thereof. In some embodiments, the protein-binding ligand includes an extracellular domain (ECD) of a cell surface receptor, or an ECD of a RPTP, or a functional variant of any thereof. In some embodiments, the protein-binding ligand includes an ECD of CD47 or a functional variant thereof. In some embodiments, the first polypeptide module is operably linked to the second polypeptide module via a polypeptide linker sequence. In some embodiments, the polypeptide linker sequence comprises a glycine-serine (GS) linker or a 3C linker.

In some embodiments, a multivalent polypeptide of the disclosure include: (a) (i) a CD47 ECD, (ii) a polypeptide linker, and (iii) a CD45 scFv; (b) (i) a SIRPα scFv, (ii) a polypeptide linker; and (iii) a CD45 scFv; or (c)(i) a CD45 V_{H}H, (ii) a polypeptide linker, and (iii) a SIRPα scFv. In some embodiments, a multivalent polypeptide of the disclosure includes, in N-terminus to C-terminus direction: (a) (i) a CD47 ECD, (ii) a GS linker, and (iii) a CD45 scFv; or (b) (i) a CD47 ECD, (ii) a C3 linker, and (iii) a CD45 scFv. In some embodiments, a multivalent polypeptide of the disclosure includes, in N-terminus to C-terminus direction: (a) (i) a SIRPα scFv, (ii) a GS linker, and (iii) a CD45 scFv; or (b) (i) a SIRPα scFv, (ii) a C3 linker, and (iii) a CD45 scFv. In some embodiments, a multivalent polypeptide of the disclosure includes, in N-terminus to C-terminus direction: (a) (i) a CD45 V_{H}H, (ii) a GS linker, and (iii) a SIRPα scFv; or (b) (i) a CD45 V_{H}H, (ii) a C3 linker, and (iii) a SIRPα scFv.

In some embodiments, a multivalent polypeptide of the disclosure further includes an amino acid sequence that has at least 80% sequence identity to an amino acid sequence selected from the group consisting of SEQ ID NOS: 1-6.

In one aspect, provided herein are recombinant nucleic acid molecules comprising a nucleotide sequence encoding a multivalent polypeptide of the disclosure. In some embodiments, the nucleic acid molecules include a nucleotide sequence encoding an amino acid sequence that has at least 80% sequence identity to an amino acid sequence selected from the group consisting of SEQ ID NOS: 1-6.

In another aspect, some embodiments disclosed herein relate to a recombinant cell that includes a nucleic acid molecule as disclosed herein, and/or a multivalent polypeptide as disclosed herein. In some embodiments, the recombinant cell is a phagocytic cell. In some embodiments, the phagocytic cell is a bone marrow-derived macrophage (BMDM). In some embodiments, the phagocytic cell is a dendritic cell. In some embodiments, the dendritic cell is a bone marrow-derived dendritic cell (BMDC).

In another aspect, provided herein are methods for promoting the maturation of immature dendritic cells (DCs) in vitro, the methods include: (a) exposing immature DCs to an antigen; and (b) culturing the immature DCs in the presence of a multivalent polypeptide of the disclosure to induce the maturation of immature DCs into mature DCs. In a related aspect, also provided herein are mature DCs prepared by a method of the disclosure.

In another aspect, provided are methods for manufacturing a vaccine, the methods include: (a) exposing immature DCs to an antigen in vitro to produce a sufficient number of antigen-presenting immature DCs; and (b) promoting the maturation of the antigen-presenting immature DCs in the presence of a multivalent polypeptide of the disclosure to produce mature antigen-presenting DCs. Accordingly, vaccines manufactured by a method disclosed herein are also within the scope of the present disclosure. In some embodiments, the vaccines of the disclosure further include one or more of the following: a diluent, an excipient, an auxiliary adjuvant, a bacterial adjuvant, and/or a systemic adjuvant.

In another aspect, some embodiments of the disclosure relate to pharmaceutical compositions which include a pharmaceutical acceptable excipient and one or more of the following: (a) a multivalent polypeptide of the disclosure; (b) a recombinant nucleic acid molecule of the disclosure; (c) a recombinant cell of the disclosure; (d) a mature DC of the disclosure; and (e) a vaccine of the disclosure.

In another aspect, disclosed herein are embodiments of methods for modulating cell signaling mediated by CD47 and/or SIRPα in a subject, the methods including administering to the subject a composition that includes one or more of the following: (a) a multivalent polypeptide of the disclosure; (b) a recombinant nucleic acid molecule of the disclosure; (c) a recombinant cell of the disclosure; (d) a pharmaceutical composition of the disclosure; (e) a mature DC of the disclosure; and (f) a vaccine of the disclosure.

In yet another aspect, provided herein are methods for preventing or treating a health condition in a subject in need thereof, the method including administering to the subject a composition that includes one or more of the following: (a) a multivalent polypeptide of the disclosure; (b) a recombinant nucleic acid molecule of the disclosure; (c) a recombinant cell of the disclosure; (d) a pharmaceutical composition of the disclosure; (e) a mature DC of the disclosure; and (f) a vaccine of the disclosure.

Non-limiting exemplary embodiments of the embodiments of the methods of the disclosure can include one or more of the following features. In some embodiments, the administered composition recruits the RPTP activity to a spatial proximity of SIRPα, potentiates dephosphorylation of SIRPα, reduces SIRPα-mediated signaling, promotes macrophage phagocytosis, and/or promotes dendritic cell maturation. In some embodiments, the administered composition confers an enhancement in macrophage-mediated phagocytosis. In some embodiments, the subject is a mammal. In some embodiments, the subject has or is suspected of having a health condition associated with CD47 and/or SIRPα. In some embodiments, the health condition is a cancer or a chronic infection.

In another aspect, provided herein are methods for preventing or treating a cancer in a subject in need thereof, the method comprising: (i) culturing immature DCs to an antigen in vitro with a cancer-associated antigen or infection-associated antigen to produce antigen-presenting immature DCs; (ii) promoting the maturation of antigen-presenting immature DCs in the presence of a multivalent polypeptide as described herein to produce mature antigen-presenting DCs; and (iii) administering the subject with the produced mature antigen-presenting DCs.

In another aspect, provided herein are methods for preventing or treating a subject infected or suspected of being suspected with a parasite, a virus, a microfungus, or a bacterium, the method comprising: (i) culturing immature DCs with an antigen derived from a parasite, a virus, a microfungus, or a bacterium to produce antigen-presenting dendritic cells; (ii) promoting the maturation of dendritic cells in the presence of a multivalent polypeptide as described herein to produce mature antigen-presenting DCs; and (iii) administering the subject with the produced mature antigen-presenting DCs. In some embodiments, the subject is a mammalian subject. In some embodiments, the mammalian subject is a human.

In some embodiments, the composition is administered to the subject individually (e.g., monotherapy) or as a first therapy in combination with a second therapy (e.g., multitherapy). In some embodiments, the second therapy is selected from the group consisting of chemotherapy, radiotherapy, immunotherapy, hormonal therapy, toxin therapy, or surgery.

In another aspect, some embodiments of the disclosure relate to kits for modulating cell signaling in a subject and/or for treating a health condition in a subject in need thereof, wherein the kits instructions for use thereof and one or more of the following one or more of the following: (a) a multivalent polypeptide of the disclosure; (b) a recombinant nucleic acid molecule of the disclosure; (c) a recombinant cell of the disclosure; (d) a pharmaceutical composition of the disclosure; (e) a mature DC of the disclosure; and (f) a vaccine of the disclosure.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1B depict graphical illustration of a non-limiting example of the modulation of cellular SIRPα-mediated signaling by in cis phosphatase recruitment in accordance with some embodiments of the disclosure. Macrophage phagocytosis of target cells mediated by ON- (“don’t eat me”) versus OFF-state (“eat me”) of SIRPα.

FIG. 1A is a schematic depiction of ligand-independent (tonic) and ligand-induced signaling by the SIRPα receptor. Antibody blockade of SIRPα interaction with CD47 is expected to reduce ligand-induced but not ligand-independent signaling. Anti-SIRPα or anti-CD47 Abs thus potentiate macrophage phagocytosis of targets but not to the full extent due to the residual activity of the intracellular domain (ICD) of SIRPα.

FIG. 1B is a schematic depiction of the mechanistic basis for RIPR-mediated inhibition of SIRPα signaling. The binding of CD45 and SIRPα by SIRPα-RIPR results in recruitment of CD45 phosphatase to SIRPα on macrophages.

FIGS. 2A-2C schematically illustrate a non-limiting example of “Velcro” SIRPα-RIPR design and strategy.

FIG. 2A depicts a schematic representation of the SIRPα-RIPR molecule composed by “Velcro” CD47 ECD (Velcro).

FIG. 2B depicts the amino acid sequences of “Velcro” SIRPα-RIPR with a glycine-serine (GS) linker (GGGGTGGS; SEQ ID NO: 9), and “Velcro” SIRPα-RIPR with a 3C linker (LEVLFQGP; SEQ ID NO: 11) are shown.

FIG. 2C summarizes binding affinity of ‘“Velcro” design and wild-type (WT) CD47 ECD to SIRPα (adopted from PCT Publication No. WO2016179399A1).

FIGS. 3A-3B schematically summarize the results of experiments performed to demonstrate that SIRPα-RIPR design robustly reduces SIRPα tyrosine phosphorylation. In these experiments, HEK293 cells were transiently transfected with target receptor human HA-SIRPα, Lck and human CD45. 24 hours after transfection, cells were left untreated (lane 4) or incubated for 20 min at 37° C. with SIRPα-RIPR(GS) (lane 1, 2 and 3) or SIRPα-RIPR(3C) (lane 5, 6 and 7) to induce in cis recruitment of the CD45 intracellular domain to the intracellular domains of SIRPα. A CD45 phosphatase-deficient group was included for control purposes (CD45 dead; C853S). After lysis, chimeric receptors were immunoprecipitated with anti-HA antibody directly conjugated to magnetic beads. Samples were probed for phosphotyrosine (pTyr) and SIRPα by western blot. Data are representative of three independent biological repeats.

FIGS. 4A-4C graphically illustrate non-limiting phagocytosis assays designed to testing SIRPα-RIPR function on phagocytosis and antibody-dependent cell-mediated cytotoxicity (ADCP).

FIG. 4A is a schematic depiction of CD47-SIRPα “don’t eat me” signal axis in macrophage. In the basal state, recruitment of CD47 to tumor cells with SIRPα on macrophages results in SHP1 and 2 recruitment and activation and inhibits phagocytosis.

FIG. 4B is a schematic depiction of phagocytosis assay for testing the effect of SIRPα-RIPR. SIRPα-RIPR silencing the SIRPα signaling by recruiting a transmembrane phosphatase activity CD45, thereby disinhibiting phagocytosis.

FIG. 4C is a schematic depiction of antibody dependent cellular phagocytosis (ADCP) assay for testing the effect of SIRPα-RIPR. Again, SIRPα-RIPR design could silence the SIRPα signaling by recruiting CD45.

FIGS. 5A-5C schematically summarize the results of experiments performed to demonstrate that human SIRPα-RIPR ligands enhance Rituximab-mediated ADCP.

FIG. 5A summarizes the results of phagocytosis assays performed to test various SIRPα-RIPR polypeptides of the disclosure. In these experiments, 5 × 10⁴ human macrophages were pretreated with “Velcro” human SIRPα-RIPR(GS) or “Velcro” human SIRPα-RIPR(3C) for 30 min at 37° C., the macrophages were co-cultured with 1 × 10⁵ Raji cells (CFSE labelled) for 2 hours at 37° C. Anti CD47 was included for a positive control.

FIG. 5B summarizes the results of ADCP assay used to test a number of SIRPα-RIPR polypeptides of the disclosure. 5 × 10⁴ human macrophages were pretreated with ‘Velcro’, human SIRPα-RIPR(GS) or SIRPα-RIPR(3C) for 30 min at 37° C., 1 × 10⁵ Raji cells were pretreated with or without 5 µg/mL anti-CD20 antibody (Rituximab) for 30 min at 37° C., the macrophages were co-cultured with Raji cells for 2 hours at 37° C. The cells were harvested and stained with CD11b for 20 min at 4° C. Phagocytosis was quantified by flow cytometry. Data are mean ± SD from n = 2 biological replicates from 1 representative of 2 independent experiments.

FIG. 5C summarizes the results of PBMC macrophage phagocytosis. Human macrophages were pretreated with or without human SIRPα-RIPR for 30 min at 37° C. Raji cells were pretreated with varying Rituximab concentrations (from 0 to 5 µg/mL) for 30 min at 37° C. The macrophages were co-cultured with Raji cells for 2 hours at 37° C. Phagocytosis was quantified by flow cytometry. Data are mean ± SD from n = 2 biological replicates from 1 representative of 2 independent experiments.

FIGS. 6A-6B schematically illustrate a non-limiting example of a bispecific antibody SIRPα-RIPR design in accordance with some embodiments of the disclosure.

FIG. 6A summarizes binding affinity of SIRPα antibody AB21 with human SIRPα and mouse SIRPα from different mouse strains (adopted from PCT Publication No. WO2018057669A1).

FIG. 6B depicts the amino acid sequences of AB21, SIRPα-RIPR with a GS linker (GGGGTGGS; SEQ ID NO: 9), SIRPα-RIPR with a 3C linker (LEVLFQGP; SEQ ID NO: 11), and a schematic representation of AB21 scFv and AB21-based human SIRPα-RIPR molecules.

FIGS. 7A-7C schematically summarize the results of experiments performed to demonstrate that the AB21-based SIRPα-RIPR bispecific antibody described in FIGS. 6A-6B above can potentiate dephosphorylation of human SIRPα.

FIG. 7A is a schematic depiction of SIRPα-RIPR mechanism.

FIG. 7B is a schematic depiction of bispecific diabody having a binding affinity for CD45 and SIRPα.

FIG. 7C is a HEK293 cells were transiently transfected with human HA-SIRPα, Lck and human CD45, 24 hours after transfection, cells were left untreated (lane 1) or incubated for 20 min at 37° C. with Ab21 (lane 2 and 3) or SIRPα-RIPR(GS) (lane 4, 5) to induce in cis recruitment of the CD45 phosphatase to the intracellular domains of SIRPα. A CD45 dead group was included for control purposes. After lysis, chimeric receptors were immunoprecipitated with anti-HA antibody directly conjugated to magnetic beads. Samples were probed for pTyr and SIRPα by western blot. Data are representative of three independent biological repeats.

FIGS. 8A-8C schematically summarize the results of experiments performed to demonstrate that AB21 SIRPα-RIPR reduces SIRPα tonic signaling and enhances ADCP of human macrophages.

FIG. 8A summarizes the results of experiments performed to detect SIRPα phosphorylation after immunoprecipitation from resting THP1 macrophages. THP1 macrophages were incubated with 500 nM AB21, or SIRPα-RIPR for 30 min at 37° C. prior to SIRPα IP.

FIG. 8B is a graph illustrating in vitro antibody dependent cellular phagocytosis (ADCP) assay using macrophages isolated from human PBMCs and incubated with Raji cells pretreated with Rituximab at the indicated concentrations. Macrophage cells were incubated with target cells for 2 hours at 37° C. Phagocytosis was quantified by flow cytometry. Data are mean ± SD from n = 2 biological replicates from 1 representative of 2 independent experiments.

FIG. 8C is a graph illustrating in vitro antibody dependent cellular phagocytosis (ADCP) assay using macrophages isolated from human PBMCs and incubated with Raji cells pretreated with Rituximab at 1 µg/mL. Macrophage cells were incubated with target cells for 2 hours at 37° C. Phagocytosis was quantified by flow cytometry. Data are mean ± SD from n = 2 biological replicates from 1 representative of 2 independent experiments.

FIGS. 9A-9B schematically illustrate another non-limiting example of a bispecific antibody SIRPα-RIPR design in accordance with some embodiments of the disclosure.

FIG. 9A depicts the amino acid sequence of AB21, SIRPα-RIPR with a GS linker (GGGGTGGS; SEQ ID NO: 9), SIRPα-RIPR with a 3C linker ((LEVLFQGP; SEQ ID NO: 11) and schematic representation of AB21 and AB21 based mouse SIRPα-RIPR molecules.

FIG. 9B illustrates the protein analysis by size-exclusion chromatography. AB21 and SIRPα-RIPRs were expressed in Hi5 cells.

FIGS. 10A-10C schematically summarize the results of experiments performed to demonstrate that the bispecific antibody SIRPα-RIPR described in FIGS. 9A-9B above can reduce SIRPα tonic signaling and enhances ADCP of mouse macrophages.

FIG. 10A illustrates in cis recruitment of the CD45 intracellular domain to the intracellular domains of SIRPα. HEK293 cells were transiently transfected with mouse HA-SIRPα, Lck and mouse CD45, 24 hours after transfection, cells were left untreated (lane 1) or incubated for 30 min at 37° C. with Ab21 (lane 2 and 3) or SIRPα-RIPR(GS) (lane 4, 5) to induce in cis recruitment of the CD45 intracellular domain to the intracellular domains of SIRPα. A CD45 dead group was included for control purposes. After lysis, chimeric receptors were immunoprecipitated with anti-HA antibody directly conjugated to magnetic beads. Samples were probed for pTyr and SIRPα by western blot. Data are representative of three independent biological repeats.

FIG. 10B illustrates detection of SIRPα phosphorylation after immunoprecipitation from resting J774 macrophages. J774 macrophages were incubated with AB21, or mouse SIRPα-RIPR for 30 min at 37° C. prior to SIRPα IP.

FIG. 10C is a graph illustrating phagocytosis. Mouse bone marrow derived macrophage (BMDM) cells were incubated with B16F10 (CFSE labeled) pretreated with or without 2 µg/mL anti TRP-1 mAb (TA99) for 2 hours at 37° C. Mouse CD47 nanobody A4 was included for control purposes. Phagocytosis was quantified by flow cytometry. Data are mean ± SD from n = 2 biological replicates from 1 representative of 2 independent experiments.

FIGS. 11A-11B schematically summarize the results of experiments performed to demonstrate that an exemplary bispecific antibody SIRPα-RIPR described in FIGS. 9A-9B above can promote maturation of mouse bone marrow dendritic cells (BMDCs).

FIG. 11A depicts schematic representation of AB21 and mouse SIRPα-RIPR.

FIG. 11B is a graph illustrating analysis of CD86 on CD11c⁺ population. The BMDC cells were stimulated with 200 nM AB21 scFv or SIRPα-RIPR for 24 hours at 37° C. CD86 was analyzed on CD11c⁺ population by flow cytometry. A control group treated with lipopolysaccharide (LPS, 1 µg/mL) was also included for control purposes.

FIGS. 12A-12D schematically summarize the results of experiments performed to illustrated that mouse SIRPα-RIPR enhances the maturation of mouse bone marrow dendritic cells (BMDCs). FIG. 12A is a schematic depiction of BMDC differentiation. FIGS. 12B-12D show the analysis of surface expression of co-stimulatory molecules (CD83, CD86), MHC molecules (MHC-I, MHC-II), chemokine receptor CCR-7 and PD-L1 on CD11c+ population. BMDC cells were stimulated with 200 nM AB21 scFv or SIRPα-RIPR for 24 hours at 37° C. Surface expression of co-stimulatory molecules (CD83, CD86), MHC molecules (MHC-I, MHC-II), chemokine receptor CCR-7 and PD-L1 was analyzed on CD11c+ population by flow cytometry.

FIG. 13A schematically illustrates an exemplary workflow of testing SIRPα-RIPR on conventional dendritic cells (cDC2) and red pulp macrophages (RPM) in C57BL/6 spleen. In these experiments, cDC1 and RPM served as controls for studying cDC2. It was observed that cDC2 and RPM express much higher level of SIRPα than cDC1.

FIG. 13B illustrates surface expression of CCR-7 on cDC1 and cDC2. C57BL/6 mice were treated with 200 µg AB21 scFv or SIRPα-RIPR for 6 hours. Splenocytes were isolated. Surface expression of CCR-7 was analyzed on cDC1, cDC2 or RPM by flow cytometry.

FIG. 13C illustrates surface expression of CD80, CD86, MHC-I, MHC-II, PD-L1 and PD-L2 on cDC1, cDC2, and RPM. C57BL/6 mice were treated with 200 µg AB21 scFv or SIRPα-RIPR for 6 hours. Splenocytes were isolated. Surface expressions of CD80, CD86, MHC-I, MHC-II, PD-L1 and PD-L2 were analyzed on cDC1, cDC2 or RPM by flow cytometry.

FIG. 14 illustrates that SIRPα-RIPR potentiates proinflammatory cytokines production in BMDCs. 100,000 BMDC cells were stimulated with 200 nM AB21 scFv or SIRPα-RIPR for 24 hours at 37° C. IL-12 and IFNγ in supernatant were quantified by ELISA.

FIGS. 15A-15C schematically summarize that SIRPα-RIPR potentiates cross presentation of DCs.

FIG. 15A is a schematic depiction of cross presentation of ovalbumin peptide 257-264 (SIINFEKL; SEQ ID NO: 32).

FIG. 15B graphically illustrates the dose response effect of OVA257-264 peptide on OT-I cells proliferation. OT-I cells were isolated from lymph nodes of OT-I mice and purified by CD8 MACS kit. BMDC cells were pulsed with 10 pM OVA257-264 peptides for 3 hours at 37° C. 50,000 APC cells were co-cultured 1:1 with Cell Trace Violet (CTV)+ OT-I cells in the presence of 200 nM AB21 scFv or SIRPα-RIPR for 5 days. The dose response effect of OVA257-264 peptide on OT-I cells proliferation were quantified by FACS.

FIG. 15C graphically illustrates dilution of CTV in OT-I cells analyzed by flow cytometry and gated on CD3⁺ CD8⁺ population.

FIGS. 16A-16C schematically summarize that SIRPα-RIPR potentiates the capacity of BMDCs to induce OT-II cells proliferation.

FIG. 16A is a schematic depiction of antigen presentation of ovalbumin peptide 323-239 (ISQAVHAAHAEINEAGR; SEQ ID NO: 33).

FIG. 16B graphically illustrates the dose response effect of OVA323-339 peptide on OT-II cells proliferation. OT-II cells were isolated from lymph nodes of OT-II mice. BMDC cells were pulsed with 1 nM or 100 nM ovalbumin (323-339) peptide for 4 hours at 37° C. 50,000 APC cells were co-cultured 1:1 with CTV+ OT-II cells in the presence of 200 nM AB21 scFv or SIRPα-RIPR for 5 days. OT-II cells were counted by FACS.

FIG. 16C graphically illustrates dilution of CTV in OT-II cells analyzed by flow cytometry and gated on CD3⁺ CD4⁺ population.

FIGS. 17A-17C schematically summarize that SIRPα-RIPR enhances the capacity of BMDCs to induce proliferation of allogeneic T cells.

FIG. 17A is a schematic depiction of mixed-lymphocyte reaction (MLR).

FIG. 17B graphically illustrates the results of MLR. BMDCs from BALB/c mice were incubated with 50,000 allogeneic spleen T cells from C57BL/6 at different ratios in the presence of 500 nM AB21 scFv or SIRPα-RIPR. CD8+ T cells were counted by FACS.

FIG. 17C a graph illustrating dilution of CTV in CD8⁺ T cells analyzed by flow cytometry and gated on CD3⁺ CD8⁺ population.

FIG. 18 illustrates that SIRPα-RIPR potentiates the antitumor response in KP1 lung cancer. F1 mice were implanted with 1×10⁶ KP1 cells and then treated with 200 µg AB21 scFv (n=5), SIRPα-RIPR (n=5) or anti-CD47 (n=5) every other day starting from Day 9. Tumor size was measured starting from Day 8.

FIGS. 19A-19B schematically summarize that SIRPα-RIPR enhances the infiltration of tumor associated macrophages in KP1 tumor.

FIG. 19A graphically illustrates tumor infiltrating lymphocytes isolated by Ficoll and stained for pan macrophage markers F4/80 and CD11b (n=10).

FIG. 19B graphically illustrates quantification of CD206⁺ tumor associated macrophages by FACS (n=10).

FIGS. 20A-20B schematically summarize that SIRPα-RIPR enhances the DC maturation in KP1 tumor.

FIG. 20A is a graph illustrating tumor infiltrating lymphocytes stained for DC marker CD11c (n=10).

FIG. 20B graphically illustrates quantification of CD86⁺ DCs by FACS (n=10).

### DETAILED DESCRIPTION OF THE DISCLOSURE

The present disclosure generally relates to, inter alia, compositions and methods for modulating cell surface receptor signaling by specifically recruiting a membrane phosphatase activity to the spatial proximity of the inhibitory receptor SIRPα (also known as CD172a, SHPS-1, or BIT). This method for inhibiting SIRPα receptor signaling represents an alternative approach to ECD ligand blockade. More particularly, the disclosure provides novel multivalent protein-binding molecules that specifically bind the SIRPα receptor and antagonize the receptor’s signaling, either completely or partially, through recruitment of a phosphatase activity, e.g., a transmembrane phosphatase. This approach, termed “Receptor Inhibition by Phosphatase Recruitment” (RIPR), was described previously in, for example, WO2019/222547A1. In particular, the experimental data presented herein has demonstrated that disruption of SIRPα signaling, by either anti-SIRα antibodies or RIPR constructs targeting SIRPα (“RIPR-SIRPα”) results in signal inhibition and promotion of macrophage phagocytosis.

As described in greater detail below, a number of RIPR molecules capable of targeting SIRPα have been designed, constructed, and subsequently evaluated for their ability to enhance macrophage activity in vitro. Blockade of SIRPα interaction with CD47 has been shown to potentiate antibody dependent phagocytosis (ADCP) and antibody dependent cellular cytotoxicity (ADCC).

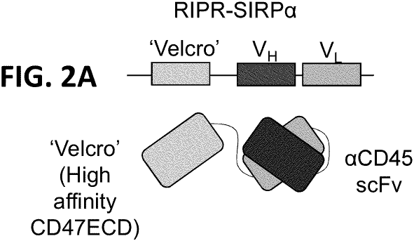

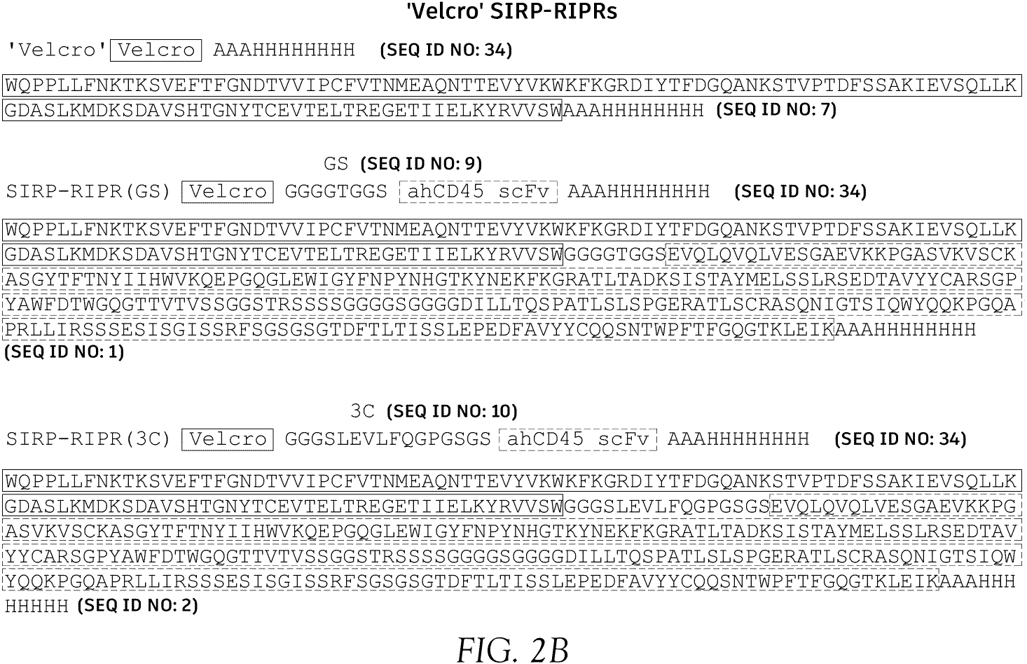

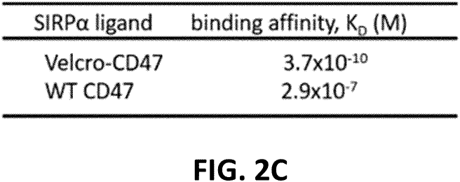

In some embodiments, to construct an exemplary RIPR molecule capable of binding SIRPα, a previously engineered high affinity CD47 ectodomain, named Velcro (Ho C.C. et al., J. Biol. Chem. 290, 12650-12663, 2015) was fused to an anti-CD45 scFv (see, e.g., FIGS. 2A-2B and Example 2). In an in vitro co-culture assay performed with human macrophages, Raji B cells and Rituximab (anti-CD20 antibody), some RIPR-SIRPα molecules of the disclosure were observed to enhance ADCP to higher levels than those achieved by a control sample containing Velcro alone. In addition, treatment with 3C was found to eliminate the RIPR effect.

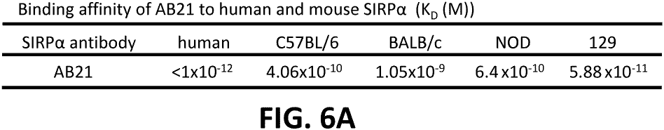

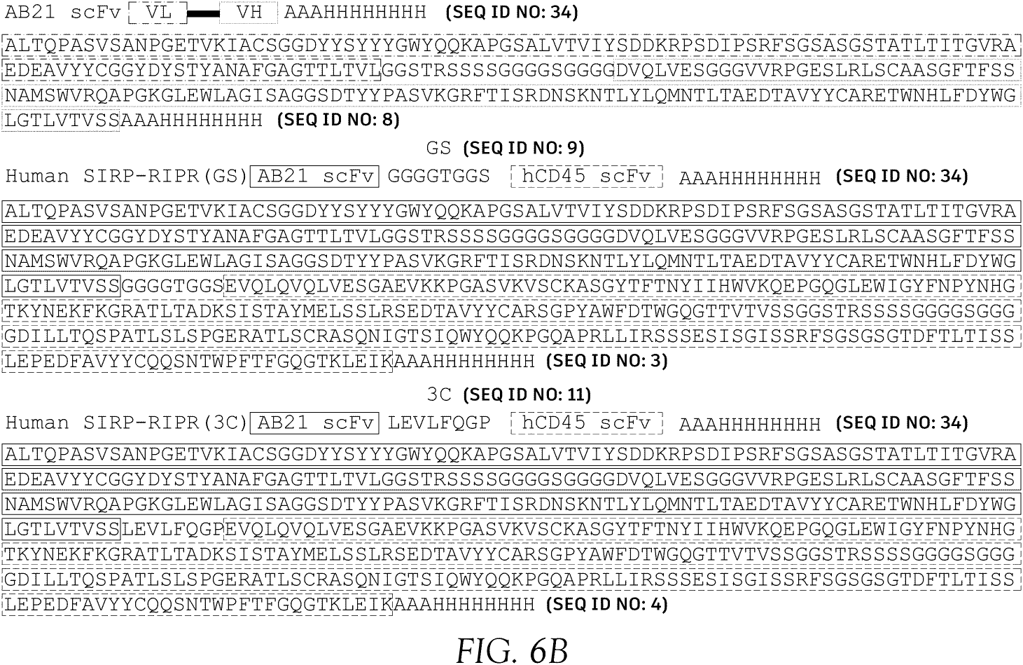

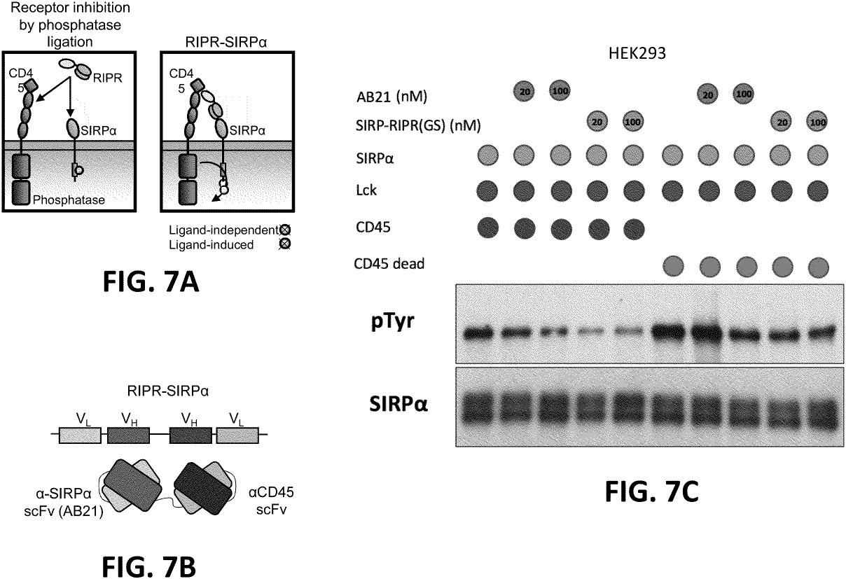

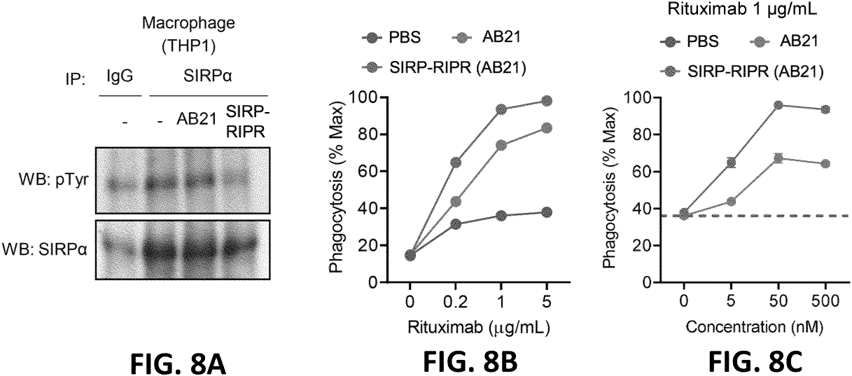

In some experiments described below, a second exemplary design of RIPR-SIRPα molecule was generated and composed of an anti-SIRPα blocking scFv which has been shown to enhance ADCP (clone AB21; FIGS. 6A-6B). This exemplary RIPR-SIRPα molecule was capable to reduce SIRPα phosphorylation in a reconstitution assay in HEK293 cells that were transiently transfected with SIRPα, Lck and CD45 (see, FIG. 7C). As shown in FIG. 7C, in “resting” macrophages, RIPR-SIRPα, but not AB21, also reduced SIRPα phosphorylation. Furthermore, it was also observed that RIPR-SIRPα was more potent than AB21 at enhancing phagocytosis after co-incubation of macrophages and Raji B cells with Rituximab, a chimeric monoclonal antibody against the CD20, which is primarily found on the surface of immune system B cells. RIPR-SIRPα induced higher phagocytosis for a wide range of Rituximab or AB21 concentrations (see, e.g., FIGS. 8A-8C). Taken together, these results indicate that recruitment of phosphatase CD45 to a SIRPα molecule present in the same cell (i.e., in cis) can be used to reduce receptor signaling of one more targets of interest.

In some embodiments, the recruitment of phosphatase is achieved via physical ligation. In some embodiments of the disclosure, the multivalent protein-binding molecules are multivalent polypeptides (e.g., bivalent or trivalent) including a first polypeptide fragment capable of binding to a receptor protein-tyrosine phosphatase (RPTP), and a second polypeptide fragment capable of binding to the SIRPα receptor which signals through a phosphorylation mechanism. The disclosure also relates to compositions and methods useful for producing such multivalent protein-binding molecules, as well as methods for the treatment of health conditions associated with the inhibition of signal transduction mediated by SIRPα.

As described in greater detail below, the present disclosure provides for, inter alia, engineered multivalent polypeptides, each exhibiting binding affinity to at least two cellular targets: a RPTP and a SIRPα molecule. Without being bound by any particular theory, it is believed that the multivalent polypeptides disclosed herein are capable of recruiting the phosphatase activity encoded by RPTP to the spatial proximity of the SIRPα molecule, subsequently reduces its phosphorylation. It is also believe that the multivalent molecule facilitates the modulation of the activity of a SIRPα molecule by binding to the extracellular domain of the SIRPα and the extracellular domain of a transmembrane phosphatase such that the intracellular domains of the SIRPα molecule and phosphatase are brought into sufficiently close proximity such that intracellular domain of the phosphatase dephosphorylates the intracellular domain of the SIRPα (or associated phosphorylated molecules), thereby reducing the activity of the SIRPα molecule. In the case where the RPTP is CD45, ligation of a module which binds to the extracellular domain of the SIRPα molecule to a module which binds to the extracellular domain of the receptor protein-tyrosine phosphatase CD45 results in dephosphorylation of SIRPα, reduces SIRPα tonic signaling, and enhances ADCP of macrophages. It is also believed that, without being bound by any particular theory, reducing the activity of SIRPα is expected to enhance ADCP of macrophages and is useful as a therapy for a wide range of diseases, including cancer and chronic infection. This novel approach bypasses the current traditional strategy of regulating cellular receptor function through ligand blockade and allows regulating cellular receptor function by dephosphorylation of the receptor intracellular domain(s).

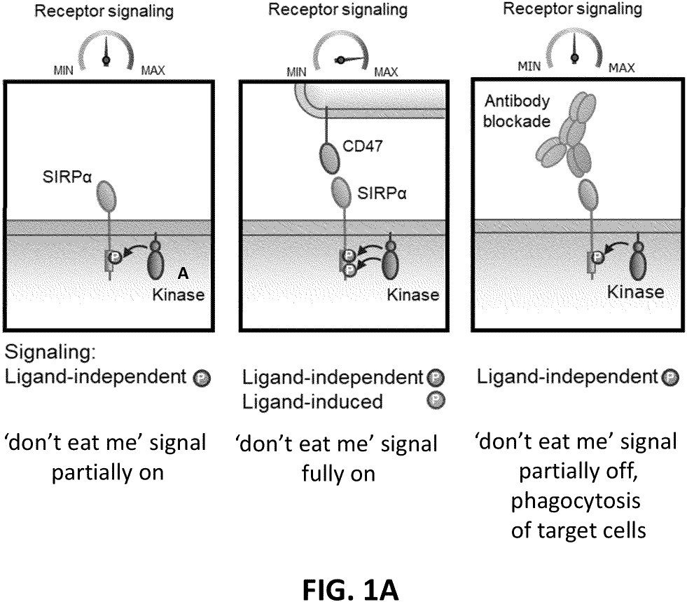

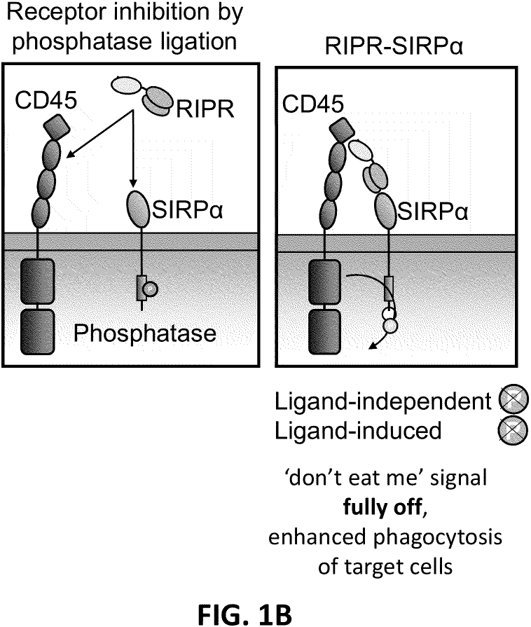

As discussed in greater detail below, it has been recognized that the current clinical options to modulate cell surface receptors is limited to ECD blocking antibodies, which block a receptor-ligand interaction from occurring at the surface of the cell. For example, in the case of SIRPα, blocking the extracellular SIRPα/CD47 interaction with high affinity antibodies has, to date, been the only available means to reduce SIRPα signaling. However, antibody blocking does not directly affect SIRPα phosphorylation and, importantly, does not reverse the basal, tonic, phosphorylation of SIRPα and sustained SIRPα from past interactions with CD47 (see, e.g., FIG. 1A). As described in greater detail below, antibody blockade of SIRPα interaction with CD47 is expected to reduce ligand-induced but not ligand-independent signaling. Anti-SIRPα or anti-CD47 antibodies thus potentiate macrophage phagocytosis of targets but not to the full extent due to the residual activity of the SIRPα intracellular domain (ICD). Without being bound by any particular theory, it is believed that existing blocking antibodies are not capable of completely eliminating SIRPα. basal signaling in order to recover full T-cell activity. As described in some embodiments of the present disclosure, newly engineered multivalent antibodies address this problem by directly recruiting a phosphatase to dephosphorylate SIRPα. For example, the present disclosure shows that CD45 recruitment is able to eliminate the exhausted phenotype induced by SIRPα, in the presence or absence of CD47. Accordingly, recruitment of phosphatases, and in particular of CD45, to receptors of interest represents a novel way to modulate the activity of cell surface receptors of interest.

The methods disclosed herein implemented the previously disclosed RIPR technology to target a cell surface receptor SIRPα, which is the receptor for CD47. Antagonists of the CD47/SIRPα axis are in clinical trial for cancer and other diseases. Currently antibodies to both SIRPα and CD47 are being evaluated clinically. One possible advantage of targeting SIRPα is that it is expressed only on macrophages, whereas CD47 is expressed on most tissues.

While SIRPα antagonism blocks the “don’t eat me” signal effectively, for reasons explained above, SIRPα and other ITAM/ITIM/ITSM have some levels of residual receptor signal even when not bound to their ligands. This phenomenon is called tonic signaling. Ligand or receptor blocking antibodies do not interfere with this tonic signaling.

As described below, the RIPR approach was employed to tether CD45 activity to SIRPα using bi-specific molecules capable of binding to both SIRPα and CD45. These newly designed SIRPα-RIPR molecules were found to inhibit the SIRPα tonic signaling. In particular, in macrophage phagocytosis assays, it was observed that SIRPα-RIPR showed increased macrophage phagocytosis of target cells over SIRPα-blocking antibodies, showing approximately a 10-20% enhancement. Thus, the data presented herein demonstrate that these SIRPα-RIPR molecules are enhanced inhibitors of the “don’t eat me” signal that can be used in clinical applications.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. The term “about”, as used herein, can have its ordinary meaning of approximately. If the degree of approximation is not otherwise clear from the context, “about” can mean either within, for example, plus or minus 10% of the provided value, or rounded to the nearest significant figure, in all cases inclusive of the provided value. Where ranges are provided, they are inclusive of the boundary values.

The terms “administration” and “administering”, as used herein, refer to the delivery of a bioactive composition or formulation by an administration route comprising, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

“Cancer” refers to the presence of cells possessing several characteristics typical of cancer-causing cells, such as uncontrolled proliferation, immortality, metastatic potential, rapid growth and proliferation rate, and certain characteristic morphological features. Cancer cells can aggregate into a mass, such as a tumor, or can exist alone within a subject. A tumor can be a solid tumor, a soft tissue tumor, or a metastatic lesion. As used herein, the term “cancer” also encompasses other types of non-tumor cancers. Non-limiting examples include blood cancers or hematological cancers, such as leukemia. Cancer can include premalignant, as well as malignant cancers.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

As used herein, the term “multivalent polypeptide” as used herein refers to a polypeptide comprising two or more protein-binding modules that are operably linked to each other. For example, a “bivalent” polypeptide of the disclosure includes two protein-binding modules, whereas a “trivalent” polypeptide of the disclosure includes three protein-binding modules. The amino acid sequences of the polypeptide modules may normally exist in separate proteins that are brought together in the multivalent polypeptide or they may normally exist in the same protein but are placed in a new arrangement in the multivalent polypeptide. A multivalent polypeptide may be created, for example, by chemical synthesis, or by creating and translating a polynucleotide in which the peptide regions are encoded in the desired relationship.

As used herein, and unless otherwise specified, a “therapeutically effective amount” or a “therapeutically effective number” of an agent is an amount or number sufficient to provide a therapeutic benefit in the treatment or management of a disease, e.g., cancer, or to delay or minimize one or more symptoms associated with the disease. A therapeutically effective amount or number of a compound means an amount or number of therapeutic agent, alone or in combination with other therapeutic agents, which provides a therapeutic benefit in the treatment or management of the disease. The term “therapeutically effective amount” can encompass an amount or number that improves overall therapy of the disease, reduces or avoids symptoms or causes of the disease, or enhances therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human subject) and non-human animals. In some embodiments, a “subject” or “individual” is a patient under the care of a physician. Thus, the subject can be a human patient or a subject who has, is at risk of having, or is suspected of having a disease of interest (e.g., cancer) and/or one or more symptoms of the disease. The subject can also be a subject who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all subcombinations of the various embodiments and elements thereof are also specifically embraced by the present disclosure and are disclosed herein just as if each and every such sub- combination was individually and explicitly disclosed herein.

### CD45 and the R1/R6 Subtype of RPTPs

Reversible protein tyrosine phosphorylation is a major mechanism regulating cellular signaling that affects fundamental cellular events including metabolism, proliferation, adhesion, differentiation, migration, communication, and adhesion. For example, protein tyrosine phosphorylation determines protein functions, including protein-protein interactions, conformation, stability, enzymatic activity and cellular localization. Disruption of this key regulatory mechanism contributes to a variety of human diseases including cancer, diabetes, and auto-immune diseases. Net protein tyrosine phosphorylation is determined by the dynamic balance of the activity of protein tyrosine kinases (PTKs) and protein tyrosine phosphatases (PTPs). Aberrant regulation of the delicate balance between PTKs and PTPs is involved in the pathogenesis of a number of human diseases such as cancer, diabetes, and autoimmune diseases.

The PTPs can be further sub-divided into transmembrane receptor-like PTPs (RPTPs) and non-transmembrane PTPs based on their overall structure. Of these, receptor-type protein tyrosine phosphatases (RPTPs) are a family of integral cell surface proteins that possess intracellular PTP activity, and extracellular domains (ECDs) that have sequence homology to cell adhesion molecules (CAMs). Intracellular domains (ICDs) of most of the RPTPs contain two tandem PTP domains, termed D1 and D2. Generally, membrane proximal PTP domain (D1) possesses most of the catalytic activity, whereas membrane-distal PTP domain (D2) has weak, if any, catalytic activity. The ECDs of RPTPs contain combinations of CAM-like motifs with sequences homologous to fibronectin type III (FN3), meprin, A5, PTPµ (MAM), immunoglobulin (Ig), and carbonic anhydrase (CA). Collectively, the molecular structure of RPTPs enables direct coupling of extracellular adhesion-mediated events to regulation of intracellular signaling pathways.

Based on the structure of their ECDs, the RPTP family can be grouped into eight sub-families: R1/R6, R2A, R2B, R3, R4, R5, R7, and R8. Representative members of these sub-families include CD45, LAR, RPTP-κ, DEP1, RPTP-α, RPTP-ζ, PTPRR, and IA2, respectively. Further information regarding the structural features that define each of the sub-families, their molecular/biochemical structure, mode of regulation, substrate specificity, and biological functions has been extensively documented and can be found in, e.g., Xu Y. et al. (J. Cell Commun. Signal. 6:125, 138, 2012). Without being bound to any particular theory, any phosphatase present in lymphocytes with an extracellular region (RPTP) could be used for the RIPR technology, with expected varying degrees of efficiency.

The receptor type protein tyrosine phosphatase CD45, also called the leukocyte common antigen (LCA), is a member of the R1/R6 subfamily of RPTPs. CD45 is a type I transmembrane protein that is in various forms present on all differentiated hematopoietic cells, except erythrocytes and plasma cells, and assists in the activation of those cells (a form of co-stimulation). CD45 is expressed in lymphomas, B-cell chronic lymphocytic leukemia, hairy cell leukemia, and acute nonlymphocytic leukemia. Human CD45, which is encoded by the gene PTPRC, is a cell membrane tyrosine phosphatase expressed by all cells of lymphoid origin, including hematopoietic cells, with the exception of platelets and erythrocytes, and functions as a key regulator of T and B cell signaling. CD45 consists of an extracellular region, short transmembrane segment and tandem PTP domains in the cytoplasmic region. Multiple isoforms of CD45 are generated by complex alternative splicing of exons in the extracellular domain of the molecule, which are expressed in a cell type specific manner depending on the cell differentiation and activation status. Non-limiting examples of CD45 isoforms include CD45RA, CD45RB, CD45RC, CD45RAB, CD45RAC, CD45RBC, CD45R0, CD45R (ABC). CD45RA is located on naive T cells and CD45R0 is located on memory T cells. CD45R is the longest protein and migrates at 200 kDa when isolated from T cells. B cells also express CD45R with heavier glycosylation, bringing the molecular weight to 220 kDa, hence the name B220; B cell isoform of 220 kDa. B220 expression is not restricted to B cells and can also be expressed on activated T cells, on a subset of dendritic cells and other antigen-presenting cells. Naive T lymphocytes express large CD45 isoforms and are usually positive for CD45RA. Activated and memory T lymphocytes express the shortest CD45 isoform, CD45R0, which lacks RA, RB, and RC exons. This shortest isoform is believed to facilitate T-cell activation. In principle, all of the CD45 isoforms discussed above can be suitable for the methods and compositions disclosed herein. Without being bound to any particular theory, it is believed that the intracellular CD45 phosphatase is not affected by the different isoforms, therefore from the intracellular point of view, all CD45 isoforms are essentially the same.

CD45 plays important roles in immune system development and function and is required for antigen-specific lymphocyte stimulation and proliferation. CD45 regulates immune responses by controlling the TCR activation threshold, modulating cytokine responses, and regulating lymphocyte survival. All of these processes are essential in the pathogenesis of autoimmune and infectious diseases.

CD45 is a suitable RPTP target for being recruited to cell surface receptors such as SIRPα, because it will act on a broad range of substrates if they are brought into a spatial proximity of one to another, e.g. the two RPTP-binding and SIRPα-binding modules are in sufficient proximity to achieve dephosphorylation of the intracellular domain of the SIRPα molecule. CD45 mediates T- and B-cell receptor function by regulating tyrosine phosphorylation of the Src family of PTKs (SFKs) like Lyn and Lck. CD45 dephosphorylates the inhibitory C-terminal phosphorylation site in Lyn and Lck, thereby potentiating the activity of these SFKs. Attenuation of SFK activity by CD45-mediated dephosphorylation of other tyrosines has also been reported. Studies in CD45 knockout mice show that CD45-mediated activation of Fyn and Lck is important in thymocyte development. Upon TCR ligation, activated Fyn and Lck phosphorylate components of the TCR complex like TCR-zeta and CD3-epsilon. These tyrosine-phosphorylated proteins provide docking sites for Src-homology 2 (SH2) domain-containing proteins to transmit down-stream signal. In CD45-null thymocytes, ligation of TCR does not lead to Lyn or Lck activation or to subsequent tyrosine phosphorylation of the TCR complex. Therefore, none of the down-stream signaling events occur; indicating the essential role of CD45 in TCR activation. CD45 has also been identified as a PTP that dephosphorylates the CD3-zeta and CD3-epsilon ITAMs, Janus kinases (JAKs) and negatively regulates cytokine receptor activation.

### Signal Regulatory Protein Α (SIRPα) and CD47

SIRPα (also known as CD172a, SHPS-1, or BIT) is a regulatory membrane glycoprotein belonging to SIRP family expressed mainly by macrophages. SIRPα acts as inhibitory receptor and interacts with a broadly expressed transmembrane protein CD47, an anti-phagocytic signal that distinguishes live cells from dying cells, which is also called the “don’t eat me” signal. This interaction negatively controls effector function of innate immune cells such as host cell phagocytosis. The interaction between SIRPα and CD47 can be modified by endocytosis, or cleavage of the SIRPα receptor, or interaction with surfactant proteins A and D. Surfactant protein A and D are soluble ligands, highly expressed in the lung, that bind to the same region of SIRPα as CD47 and can therefore competitively block binding. SIRPα diffuses laterally on the macrophage membrane and accumulates at a phagocytic synapse to bind CD47 and signal “self”, which inhibits the cytoskeleton-intensive process of phagocytosis by the macrophage.

The cytoplasmic region of SIRPα is highly conserved between rats, mice and humans. Cytoplasmic region contains a number of tyrosine residues, which likely act as ITIMs. Upon CD47 binding, SIRPα is phosphorylated and recruits phosphatases like SHP1 and SHP2. The extracellular region contains three Immunoglobulin superfamily domains -single V-set and two C1-set IgSF domains. SIRPβ and SIRPγ have the similar extracellular structure but different cytoplasmic regions giving contrasting types of signals. SIRPα polymorphisms are found in ligand-binding IgSF V-set domain but it does not affect ligand binding.

In SIRPα-mediated signaling, the extracellular domain of SIRPα binds to CD47 and transmits intracellular signals through its cytoplasmic domain (e.g., intracellular domain). CD47-binding is mediated through the NH₂-terminal V-like domain of SIRPα. The cytoplasmic region contains four ITIMs that become phosphorylated after binding of ligand. The phosphorylation mediates activation of tyrosine kinase SHP2. SIRPα has been shown to bind also phosphatase SHP1, adaptor protein SCAP2 and FYN-binding protein. Recruitment of SHP phosphatases to the membrane leads to the inhibition of myosin accumulation at the cell surface and results in the inhibition of phagocytosis. The SIRPα / CD47 interaction is unusual in that it can lead to bidirectional signaling through both SIRPα and CD47. Engagement of SIRPα by CD47 provides a down regulatory signal that inhibits host cell phagocytosis, and CD47 therefore functions as a “don’t-eat-me” signal to macrophages of the immune system which has made it a potential therapeutic target in some cancers, and more recently, for the treatment of pulmonary fibrosis. CD47 is involved in a range of cellular processes, including apoptosis, proliferation, adhesion, and migration. Furthermore, it plays a key role in immune and angiogenic responses. CD47 is ubiquitously expressed in human cells and has been found to be overexpressed in many different tumor cells. Expression in equine cutaneous tumors has been reported as well.

In addition, SIRPα also plays an inhibitory function of signal regulatory in dendritic cell survival and activation. In tumors from human liver cancer patients, infiltrative DCs expressed elevated levels of SIRPα, which is correlated with the induction of immune tolerance within the tumors. Silencing of SIRPα resulted in a significant increase in the longevity of antigen-pulsed DCs in the draining lymph nodes. Furthermore, SIRPα controls the activation and output of DCs. Silencing of DC-expressed SIRPα induced spontaneous and enhanced production of IL-12 and costimulatory molecules, resulting in more potent cytotoxic T lymphocyte responses, including the eradication of previously established solid tumors. SIRPα exerted such effects, at least in part, via the association and sequestration of p85 subunit of PI3K. Thus, SIRPα is widely considered to be an important regulator of DC lifespan and activity, and its inhibition can be used in improving the clinical efficacy of DC-based tumor vaccines. More information in this regard can be found in, for example, Liu et al., Oncoimmunology, 2016,.

### Compositions of the Disclosure

As described in greater detail below, one aspect of the present disclosure relates to multivalent protein-binding molecules that specifically bind the SIRPα receptor and antagonize the receptor’s signaling through recruitment of a RPTP activity, e.g., transmembrane phosphatase CD45. Also provided are (i) recombinant nucleic acids encoding such multivalent protein-binding molecules, (ii) recombinant cells that have been engineered to express a multivalent protein-binding molecule as disclosed herein.

### Multivalent Polypeptides and Multivalent Antibodies

In one aspect, some embodiments disclosed herein relate to a novel chimeric polypeptides containing multiple polypeptide modules, e.g., modular protein-binding moieties, each capable of binding to one or more target protein(s). In some embodiments, the disclosed chimeric polypeptide includes (i) a first amino acid sequence including a first polypeptide module capable of binding to a SIRPα molecule, and (ii) a second amino acid sequence including a second polypeptide module capable of binding to one or more RPTPs of R1/R6 subfamily. In some embodiments, the first polypeptide module is operably linked to the second polypeptide module. In some embodiment, the disclosed chimeric polypeptide is a multivalent polypeptide. In some embodiment, the multivalent polypeptide is a multivalent antibody. The binding of a first polypeptide module and a second polypeptide module to their respective target can be either in a competitive or non-competitive fashion with a natural ligand of the target. Accordingly, in some embodiments of the disclosure, the binding of a first polypeptide module and/or second polypeptide module to their respective target can be ligand-blocking. In some other embodiments, the binding of a first polypeptide module and/or second polypeptide module to their respective target does not block binding of the natural ligand.

Designation of the amino acid sequence of the multivalent polypeptide that includes a first polypeptide module capable of binding to a SIRPα molecule as the “first” amino acid sequence and the amino acid sequence of the multivalent polypeptide including a polypeptide module capable of binding to a RPTP as the “second” amino acid sequence is not intended to imply any particular structural arrangement of the “first” and “second” amino acid sequences within the multivalent polypeptide. By way of non-limiting example, in some embodiments of the disclosure, the multivalent polypeptide or multivalent antibody may include an N-terminal polypeptide module capable of binding to a SIRPα molecule and a C-terminal polypeptide module including a polypeptide capable of binding to a RPTP. In other embodiments, the multivalent polypeptide or multivalent antibody may include an N-terminal polypeptide module capable of binding to a RPTP and a C-terminal polypeptide module capable of binding to a SIRPα molecule. In addition or alternatively, the multivalent polypeptide or multivalent antibody may include more than one polypeptide module capable of binding to a SIRPα, and/or more than one polypeptide module capable of binding to a RPTP. Accordingly, in some embodiments, a first amino acid sequence of the multivalent polypeptide or multivalent antibody includes at least two, three, four, five, six, seven, eight, nine, or ten polypeptide modules each capable of binding to a SIRPα. In some embodiments, the at least two, three, four, five, six, seven, eight, nine, or ten polypeptide modules of a first amino acid sequence are each capable of binding to the same RPTP. In some embodiments, the at least two, three, four, five, six, seven, eight, nine, or ten polypeptide modules of a first amino acid sequence are each capable of binding to different RPTPs.

In addition or alternatively, as alluded to above, the multivalent polypeptides and antibodies as disclosed herein can incorporate both natural and unnatural amino acids at positions that affect the binding affinity of the multivalent polypeptides or multivalent antibodies with the respective target protein(s). As such, the binding affinity of the polypeptide modules to their respective target (e.g., RPTP or SIRPα) can be tuned to achieve a desired target cell specificity. For example, since CD45 is widely expressed, the SIRPα-binding module can be configured to form a high affinity binding module, while the CD45-binding module can be configured to have lower binding affinity. For instance, in some embodiments, a SIRPα-binding module has a higher affinity (lower K_{d}) to the SIRPα when compared to the binding affinity of the RPTP-binding module to the RPTP. In some embodiments, the difference in affinity is at least one order of magnitude or at least two orders of magnitude (e.g., the ratio of the K_{d} for the interaction of the RPTP-binding module to the RPTP to the K_{d} for the interaction of the SIRPα-binding module to the SIRPα is at least 10, at least 20, at least 50, or at least 100). One skilled in the art will appreciate that this concept of a multivalent polypeptide or multivalent antibody having high affinity for the RPTP or its target receptor (e.g., SIRPα), and lower affinity for the other can be an important part of tuning RIPR activity for target cell specificity. Accordingly, in some embodiments, the binding affinity of the RPTP-binding polypeptide module can be different from the binding affinity of the SIRPα-binding polypeptide module. For example, in some embodiments, the RPTP-binding polypeptide module has high affinity to its target (e.g., RPTP) and the SIRPα-binding polypeptide module has low affinity to its target (e.g., SIRPα). In some embodiments, the RPTP-binding polypeptide module has low affinity to its target and the SIRPα-binding polypeptide module has high affinity to its target. In some embodiments, the RPTP-binding and SIRPα-binding modules have the same affinity to the respective target proteins.

In some embodiments, the binding affinity of the SIRPα-binding and RPTP-binding modules each having an affinity for the extracellular domain of its respective target, is independently from K_{d} =10⁻⁵ to 10⁻¹² M, such as e.g., a K_{d} of about 10⁻⁵ to about 10⁻¹¹ M, alternatively a K_{d} of about 10⁻⁵ to about 10⁻¹⁰ M, alternatively a K_{d} of about 10⁻⁶ to about 10⁻ ¹² M, alternatively a K_{d} of about 10⁻⁷ to about 10⁻¹² M, alternatively a K_{d} of about 10⁻⁸ to about 10⁻¹² M, alternatively a K_{d} of about 10⁻⁹ to about 10⁻¹² M, alternatively a Kd of about 10⁻¹⁰ to about 10⁻¹² M, alternatively a K_{d} of about 10⁻¹¹ to about 10⁻¹² M, alternatively a K_{d} of about 10⁻⁵ to about 10⁻¹¹ M, alternatively a K_{d} of about 10⁻⁵ to about 10⁻¹⁰ M, alternatively a K_{d} of about 10⁻⁵ to about 10-⁹ M, alternatively a K_{d} of about 10⁻⁵ to about 10⁻⁸ M, alternatively a K_{d} of about 10⁻⁵ to about 10⁻⁷ M, alternatively a K_{d} of about 10⁻⁵ to about 10⁻⁶ M.

In some embodiments, the multivalent polypeptide or multivalent antibody as disclosed herein has a binding affinity for a RPTP (e.g., CD45) with a K_{d} of about 1,000 nM, about 800 nM, about 700 nM, about 600 nM, about 500 nM, about 400 nM, about 200 nM, about 100 nM, about 10 nM, about 5 nM, or about 1 nM. In some embodiments, the multivalent polypeptide or multivalent antibody as disclosed herein have low binding affinity for a RPTP, e.g. with a K_{d} of more than about 10⁻⁵ M, such as e.g., a K_{d} of more than about 10⁻⁴ M, more than about 10⁻³ M, more than about 10⁻² M, or more than about 10⁻¹ M. In some embodiments, the binding affinity (Kd) for a RPTP (e.g., CD45) can be about 700 nM. In some embodiments, the binding affinity of the multivalent polypeptide or multivalent antibody for CD45 can be about 300 nM.

In some embodiments, the multivalent polypeptide or multivalent antibody as disclosed herein can have binding affinity for a SIRPα molecule with a K_{d} of 1,000 nM, about 800 nM, about 700 nM, about 600 nM, about 500 nM, about 400 nM, about 200 nM, about 150 nM, about 100 nM, about 80 nM, about 60 nM, about 40 nM, about 20 nM, about 10 nM, about 5 nM, or about 1 nM. In some embodiments, the multivalent polypeptide or multivalent antibody as disclosed herein has a high binding affinity for a SIRPα molecule, e.g. with a K_{d} of less than about 10⁻⁸ M, less than about 10-⁹ M, less than about 10⁻¹⁰ M, less than about 10⁻¹¹ M, or less than about 10⁻¹² M. In some embodiments, the binding affinity of a multivalent polypeptide or multivalent antibody disclosed herein for a SIRPα molecule has a K_{d} of about 7 nM. In some embodiments, the binding affinity of a multivalent polypeptide or multivalent antibody disclosed herein for a SIRPα molecule has a K_{d} of about 6 nM. In some embodiments, the binding affinity for a SIRPα molecule can be about 5 nM.

In some embodiments, a first amino acid sequence of the multivalent polypeptide or multivalent antibody is directly linked to a second amino acid sequence. In some embodiments, a first amino acid sequence is directly linked to a second amino acid sequence via at least one covalent bond. In some embodiments, a first amino acid sequence is directly linked to a second amino acid sequence via at least one peptide bond. In some embodiments, the C-terminal amino acid of a first amino acid sequence can be operably linked to the N-terminal amino acid of a second polypeptide module. Alternatively, the N-terminal amino acid of a first polypeptide module can be operably linked to the C-terminal amino acid of a second polypeptide module.

In some embodiments, a first amino acid sequence of the multivalent polypeptide or multivalent antibody is operably linked to a second amino acid sequence via a linker. There is no particular limitation on the linkers that can be used in the multivalent polypeptides described herein. In some embodiments, the linker is a synthetic compound linker such as, for example, a chemical cross-linking agent. Non-limiting examples of suitable cross-linking agents that are commercially available include N- hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES). Other examples of alterative structures and linkages suitable for the multivalent polypeptides and multivalent antibodies of the disclosure include those described in Spiess et al., Mol. Immunol. 67:95-106, 2015.

In some embodiments, a first amino acid sequence of a multivalent polypeptide or multivalent antibody disclosed herein is operably linked to a second amino acid sequence via a linker polypeptide sequence (peptidal linkage). In principle, there are no particular limitations to the length and/or amino acid composition of the linker polypeptide sequence. In some embodiments, any arbitrary single-chain peptide comprising about one to 100 amino acid residues (e.g., 2, 3, 4, 5, 6, 7, 8, 9, 10, 11, 12, 13, 14, 15, 16, 17, 18, 19, 20, etc. amino acid residues) can be used as a polypeptide linker. In some embodiments, the linker polypeptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker polypeptide sequence includes about 1 to 10, about 5 to 15, about 10 to 20, about 15 to 25, about 20 to 40, about 30 to 50, about 40 to 60, about 50 to 70 amino acid residues. In some embodiments, the linker polypeptide sequence includes about 40 to 70, about 50 to 80, about 60 to 80, about 70 to 90, or about 80 to 100 amino acid residues. In some embodiments, the linker polypeptide sequence includes about 1 to 10, about 5 to 15, about 10 to 20, about 15 to 25 amino acid residues.

In some embodiments, the length and amino acid composition of the linker polypeptide sequence can be optimized to vary the orientation and/or proximity of a first and a second polypeptide modules relative to one another to achieve a desired activity of the multivalent polypeptide. In some embodiments, the orientation and/or proximity of a first and a second polypeptide modules relative to one another can be varied as a “tuning” tool to achieve a tuning effect that would enhance or reduce the RPTP activity of the multivalent polypeptide. In some embodiments, the orientation and/or proximity of a first and a second polypeptide modules relative to one another can be optimized to create a partial antagonist to full antagonist versions of the bispecific polypeptide. In certain embodiments, the linker contains only glycine and/or serine residues (e.g., glycine-serine linker). Examples of such polypeptide linkers include: Gly, Ser; Gly Ser; Gly Gly Ser; Ser Gly Gly; Gly Gly Gly Ser (SEQ ID NO: 12); Ser Gly Gly Gly (SEQ ID NO: 13); Gly Gly Gly Gly Ser (SEQ ID NO: 14); Ser Gly Gly Gly Gly (SEQ ID NO: 15); Gly Gly Gly Gly Gly Ser (SEQ ID NO: 16); Ser Gly Gly Gly Gly Gly (SEQ ID NO: 17); Gly Gly Gly Gly Gly Gly Ser (SEQ ID NO: 18); Ser Gly Gly Gly Gly Gly Gly (SEQ ID NO: 19); (Gly Gly Gly Gly Ser)n (SEQ ID NO: 20), wherein n is an integer of one or more; and (Ser Gly Gly Gly Gly)n (SEQ ID NO: 21), wherein n is an integer of one or more. In some embodiments, the linker polypeptides are modified such that the amino acid sequence Gly Ser Gly (GSG) (that occurs at the junction of traditional Gly/Ser linker polypeptide repeats) is not present. For example, in some embodiments, the polypeptide linker includes an amino acid sequence selected from the group consisting of: (GGGXX)nGGGGS (SEQ ID NO: 22) and GGGGS(XGGGS)n (SEQ ID NO: 23), where X is any amino acid that can be inserted into the sequence and not result in a polypeptide comprising the sequence GSG, and n is 0 to 4. In some embodiments, the sequence of a linker polypeptide is (GGGX1X2)nGGGGS (SEQ ID NO: 24) and X1 is P and X2 is S and n is 0 to 4. In some embodiments, the sequence of a linker polypeptide is (GGGX1X2)nGGGGS (SEQ ID NO: 25) and X1 is G and X2 is Q and n is 0 to 4. In some other embodiments, the sequence of a linker polypeptide is (GGGX1X2)nGGGGS (SEQ ID NO: 26) and X1 is G and X2 is A and n is 0 to 4. In some embodiments, the sequence of a linker polypeptide is GGGGS(XGGGS)n (SEQ ID NO: 27), and X is P and n is 0 to 4. In some embodiments, a linker polypeptide of the disclosure comprises or consists of the amino acid sequence (GGGGA)₂GGGGS (SEQ ID NO: 28). In some embodiments, a linker polypeptide comprises or consists of the amino acid sequence (GGGGQ)₂GGGGS (SEQ ID NO: 29). In some embodiments, a linker polypeptide comprises or consists of the amino acid sequence (GGGPS)₂GGGGS (SEQ ID NO: 30). In some embodiments, a linker polypeptide comprises or consists of the amino acid sequence GGGGS(PGGGS)₂ (SEQ ID NO: 31).

In addition, or alternatively, in some embodiments, the multivalent polypeptides and multivalent antibodies of the disclosure can include one or more RPTP-binding modules chemically linked to one or more SIRPα-binding modules. In some embodiments, the multivalent polypeptides and multivalent antibodies of the disclosure can include (i) one or more RPTP-binding modules chemically linked to one or more SIRPα-binding modules; and (ii) one or more RPTP-binding modules linked to one or more SIRPα-binding modules via peptidyl linkages.

In some embodiments disclosed herein, at least one of the first and second polypeptide modules of the disclosed multivalent polypeptide or multivalent antibody includes an amino acid sequence for a protein-binding ligand or an antigen-binding moiety. In some embodiments, at least one of the first and second polypeptide modules includes an amino acid sequence for a protein-binding ligand. Generally, any suitable protein-binding ligands can be used for the compositions and methods of the present disclosure and can be, for example, any recombinant polypeptide or naturally-occurring polypeptide which has a specific binding affinity to a target antibody or a target protein (e.g., a recombinant or natural ligand of a RPTP or a SIRPα molecule) (see, also, Verdoliva et al., J. Immuno. Methods, 2002; Naik et al., J. Chromatography, 2011). For example, non-limiting examples of suitable ligands for phosphatase CD45 include its natural ligands, such as e.g., lectin CD22 (Hermiston ML et al., Annu. Rev. Immunol. 2003) and Galactin-1 (Walzel H. et al., J. Immunol. Lett. 1999 and Nguyen JT et al. J Immunol. 2001). In some embodiments, at least one of the first and second polypeptide modules of the disclosed multivalent polypeptide or multivalent antibody include an amino acid sequence for one or more extracellular domains (ECDs) of a SIRPα or of a RPTP. Accordingly, in some embodiments, a first polypeptide module of the disclosed multivalent polypeptide includes one or more ECDs of a SIRPα molecule operably linked to a second module of the multivalent polypeptide. Accordingly, in some embodiments, a first polypeptide module of the disclosed multivalent polypeptide includes one or more ECDs of CD47 operably linked to a second module of the multivalent polypeptide. In some embodiments, a second polypeptide module of the disclosed multivalent polypeptide includes one or more ECDs of a RPTP operably linked to a first module of the multivalent polypeptide.

As discussed above, non-limiting examples of protein-binding ligands suitable for the compositions and methods of the disclosure include natural ligands of SIRPα. For example, suitable natural ligands for SIRPα include CD47, as well as surfactant protein A and surfactant protein D or a fragment of any thereof, which are members of the surfactant protein family.

In addition or alternatively, the protein-binding ligand can be an agonist or an antagonist version of the target’s natural ligand. Thus, in some embodiments, the protein-binding ligand is an agonist ligand of the RPTP or the SIRPα. In some other embodiments, the protein-binding ligand is an antagonist ligand of the RPTP or the SIRPα. In some embodiments, the protein-binding ligand can be a synthetic molecule such as, for example, peptides or small molecules.

In some embodiments, at least one of a first and a second polypeptide modules of the disclosed multivalent polypeptide or multivalent antibody includes an amino acid sequence for an antigen-binding moiety that binds to the target protein, e.g., a RPTP or a SIRPα. In some embodiments, the antigen-binding moiety includes one or more antigen-binding determinants of an antibody or a functional antigen-binding fragment thereof. Blocking antibodies and non-blocking antibodies are both suitable. As used herein, the term “blocking” antibody or an “antagonist” antibody refers to an antibody that prevents, inhibits, blocks, or reduces biological or functional activity of the antigen to which it binds. Blocking antibodies or antagonist antibodies can substantially or completely prevent, inhibit, block, or reduce the biological activity or function of the antigen. For example, a blocking anti-SIRPα antibody can prevent, inhibit, block, or reduce the binding interaction between SIRPα and surfactant protein A, thus preventing, blocking, inhibiting, or reducing the immunosuppressive functions associated with the SIRPα/CD47 interaction. The term “non-blocking” antibody refers to an antibody that does not interfere, inhibits, blocks, or reduces biological or functional activity of the antigen to which it binds.

The term “antigen-binding fragment” as used herein refers to an antibody fragment such as, for example, a diabody, a Fab, a Fab′, a F(ab′)2, an Fv fragment, a disulfide stabilized Fv fragment (dsFv), a (dsFv)2, a bispecific dsFv (dsFv- dsFv′), a disulfide stabilized diabody (ds diabody), a single-chain antibody molecule (scFv), an scFv dimer (e.g., bivalent diabody -bi-scFv or divalent diabody -di-scFv), or a multispecific antibody formed from a portion of an antibody including one or more complementarity-determining regions (CDRs) of the antibody. The antigen-binding moiety can include naturally-derived polypeptides, antibodies produced by immunization of a non-human animal, or antigen-binding moieties obtained from other sources, e.g., camelids (see, e.g., Bannas et al. Front. Immunol., 22 Nov. 2017; McMahon C. et al., Nat Struct Mol Biol. 25(3): 289-296, 2018). The antigen-binding moiety can be engineered, synthesized, designed, humanized (see, e.g., Vincke et al., J. Biol. Chem. 30;284(5):3273-84, 2009), or modified so as to provide desired and/or improved properties.

Accordingly, in some embodiments, at least one of a first and a second polypeptide modules of the disclosed multivalent polypeptide or multivalent antibody includes an amino acid sequence for an antigen-binding moiety selected from the group consisting of antigen-binding fragments (Fab), single-chain variable fragments (scFv), nanobodies, V_{H} domains, V_{L} domains, single domain antibodies (dAb), V_{NAR} domains, and V_{H}H domains, diabodies, or a functional fragment of any one of the foregoing. In some embodiments, the antigen-binding moiety includes a single-chain variable fragment (scFv). In some embodiments, the antigen-binding moiety includes a diabody. In some embodiments, the antigen-binding moiety includes a bi-scFv or di-scFv, in which two scFv molecules are operably linked to each other. In some embodiments, the bi-scFv or di-scFv includes a single peptide chain with two V_{H} and two V_{L} regions, yielding tandem scFvs. In some embodiments, the antigen-binding moiety includes a nanobody. In some embodiments, the antigen-binding moiety includes a heavy chain variable region and a light chain variable region.

In some embodiments, the heavy chain variable region and the light chain variable region of the antigen-binding moiety are operably linked to each other via one or more intervening amino acid residues that are positioned between the heavy chain variable region and the light chain variable region. In some embodiments, the one or more intervening amino acid residues include a linker polypeptide sequence. In principle, there are no particular limitations to the length and/or amino acid composition of the linker polypeptide sequence. In some embodiments, any arbitrary single-chain peptide including about one to 100 amino acid residues (e.g., 2, 3, 4, 5, 6, 7, 8, 9, 10, 11, 12, 13, 14, 15, 16, 17, 18, 19, 20, etc. amino acid residues) can be used as a polypeptide linker. In some embodiments, the linker polypeptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker polypeptide sequence includes about 1 to 10, about 5 to 15, about 10 to 20, about 15 to 25, about 20 to 40, about 30 to 50, about 40 to 60, about 50 to 70 amino acid residues. In some embodiments, the linker polypeptide sequence includes about 40 to 70, about 50 to 80, about 60 to 80, about 70 to 90, or about 80 to 100 amino acid residues. In some embodiments, the linker polypeptide sequence includes about 1 to 10, about 5 to 15, about 10 to 20, about 15 to 25 amino acid residues. In some embodiments, the length and amino acid composition of the linker polypeptide sequence can be optimized to vary the orientation and/or proximity of a first and a second polypeptide modules relative to one another to achieve a desired activity of the multivalent polypeptide. In some embodiments, the orientation and/or proximity of a first and a second polypeptide modules relative to one another can be varied as a “tuning” tool or effect that would enhance or reduce the RPTP activity of the multivalent polypeptide. In some embodiments, the orientation and/or proximity of a first and a second polypeptide modules relative to one another can be optimize to create a partial antagonist to full antagonist versions of the multivalent polypeptide.

In certain embodiments, the linker contains only glycine and/or serine residues (e.g., glycine-serine linker). Examples of such polypeptide linkers include: Gly, Ser; Gly Ser; Gly Gly Ser; Ser Gly Gly; Gly Gly Gly Ser (SEQ ID NO: 12); Ser Gly Gly Gly (SEQ ID NO: 13); Gly Gly Gly Gly Ser (SEQ ID NO: 14); Ser Gly Gly Gly Gly (SEQ ID NO: 15); Gly Gly Gly Gly Gly Ser (SEQ ID NO: 16); Ser Gly Gly Gly Gly Gly (SEQ ID NO: 17); Gly Gly Gly Gly Gly Gly Ser (SEQ ID NO: 18); Ser Gly Gly Gly Gly Gly Gly (SEQ ID NO: 19); (Gly Gly Gly Gly Ser)n (SEQ ID NO: 20), wherein n is an integer of one or more; and (Ser Gly Gly Gly Gly)n (SEQ ID NO: 21), wherein n is an integer of one or more. In some embodiments, the linker polypeptides are modified such that the amino acid sequence GSG (that occurs at the junction of traditional Gly/Ser linker polypeptide repeats) is not present. For example, in some embodiments, the polypeptide linker includes an amino acid sequence selected from the group consisting of: (GGGXX)nGGGGS (SEQ ID NO: 22) and GGGGS(XGGGS)n (SEQ ID NO: 23), where X is any amino acid that can be inserted into the sequence and not result in a polypeptide including the sequence GSG, and n is 0 to 4. In some embodiments, the sequence of a linker polypeptide is (GGGX1X2)nGGGGS (SEQ ID NO: 24) and X1 is P and X2 is S and n is 0 to 4. In some other embodiments, the sequence of a linker polypeptide is (GGGX1X2)nGGGGS (SEQ ID NO: 25) and X1 is G and X2 is Q and n is 0 to 4. In some other embodiments, the sequence of a linker polypeptide is (GGGX1X2)nGGGGS (SEQ ID NO: 26) and X1 is G and X2 is A and n is 0 to 4. In yet some other embodiments, the sequence of a linker polypeptide is GGGGS(XGGGS)n (SEQ ID NO: 27), and X is P and n is 0 to 4. In some embodiments, a linker polypeptide of the disclosure comprises or consists of the amino acid sequence (GGGGA)2GGGGS (SEQ ID NO: 28). In some embodiments, a linker polypeptide comprises or consists of the amino acid sequence (GGGGQ)2GGGGS (SEQ ID NO: 29). In some embodiments, a linker polypeptide comprises or consists of the amino acid sequence (GGGPS)2GGGGS (SEQ ID NO: 30). In some embodiments, a linker polypeptide comprises or consists of the amino acid sequence GGGGS(PGGGS)2 (SEQ ID NO: 31). In some embodiments, the linker polypeptide includes a 3C protease cleavage site. Incorporation of a 3C cleavage site in the SIRPα-RIPR molecules of the disclosure allows for various cleavage experiments to be performed in which an SIRPα-RIPR molecule can be is and split into its two components, e.g., anti-SIRP binding module and anti-CD45 binding module. A SIRPα-RIPR molecule cut by 3C does not crosslink CD45 to SIRP and thus offers the possibility to test the RIPR concept. In yet some embodiments, a linker polypeptide comprises or consists of an amino acid sequence set forth in SEQ ID NOs: 9-11 in the Sequence Listing.

In some embodiments, a first polypeptide module of the multivalent polypeptides and multivalent antibodies disclosed herein includes an antigen-binding moiety capable of binding one or more target RPTPs. Non-limiting examples of suitable RPTPs include members of sub-families R1/R6. In some embodiments, a second polypeptide module of the multivalent polypeptides and multivalent antibodies disclosed herein includes an antigen-binding moiety capable of binding CD45 phosphatase or a functional variant thereof, such as e.g., a homolog thereof. In some embodiments, the CD45 phosphatase is a human CD45 phosphatase. In general, any isoforms of CD45 can be used. In some embodiments, the RPTP is a CD45 isoform selected from the group consisting of CD45RA, CD45RB, CD45RC, CD45RAB, CD45RAC, CD45RBC, CD45R0, CD45R. Exemplary CD45-binding moieties suitable for the compositions and methods disclose herein include, but are not limited to those described in U.S. Pat. Nos. 7,825,222 and 9,701,756.

Non-limiting exemplary embodiments of the multivalent polypeptide of the disclosure can include one or more of the following features. In some embodiments, the one or more RPTPs includes CD45 or a functional variant thereof. In some embodiments, at least one of the first and second polypeptide modules includes an amino acid sequence for a protein-binding ligand or an antigen-binding moiety. In some embodiments, the antigen-binding moiety is selected from the group consisting of a single-chain variable fragment (scFv), an antigen-binding fragment (Fab), a nanobody, a V_{H} domain, a V_{L} domain, a single domain antibody (dAb), a V_{NAR} domain, and a V_{H}H domain, a diabody, or a functional fragment of any thereof. In some embodiments, the protein-binding ligand includes an extracellular domain (ECD) of a SIRPα molecule, or an ECD of a RPTP, or a functional variant of any thereof. In some embodiments, the protein-binding ligand includes an ECD of CD47 or a functional variant thereof. In some embodiments, the protein-binding ligand includes “Velcro”, a high affinity CD47 or a functional variant thereof. Velcro was described previously in Ho C.C. et al., supra, 2015. In some embodiments, the first polypeptide module is operably linked to the second polypeptide module via a polypeptide linker sequence. In some embodiments, the polypeptide linker sequence comprises a glycine-serine (GS) linker. In some embodiments, the GS linker comprises or consists of SEQ ID NO: 9. In some embodiments, the polypeptide linker sequence comprises a 3C linker. In some embodiments, the 3C linker comprises or consists of SEQ ID NO: 11.

In some embodiments, a multivalent polypeptide of the disclosure include: (a) a CD47 ECD, (b) a polypeptide linker, and (c) a CD45 scFv. In some embodiments, a multivalent polypeptide of the disclosure include, in the N-terminal to C-terminal direction:

(a) a CD47 ECD, (b) a polypeptide linker, and (c) a CD45 scFv. In some embodiments, a multivalent polypeptide of the disclosure include, in the N-terminal to C-terminal direction:

(a) a CD45 scFv, (b) a polypeptide linker, and (c) a CD47 ECD.

In some embodiments, a multivalent polypeptide of the disclosure include: (a) a SIRPα scFv, (b) a polypeptide linker; and (c) a CD45 scFv. In some embodiments, a multivalent polypeptide of the disclosure include, in the N-terminal to C-terminal direction:

(a) a SIRPα scFv, (b) a polypeptide linker; and (c) a CD45 scFv. In some embodiments, a multivalent polypeptide of the disclosure include, in the N-terminal to C-terminal direction:

(a) a CD45 scFv, (b) a polypeptide linker; and (c) a SIRPα scFv.

In some embodiments, a multivalent polypeptide of the disclosure include: (a) a CD45 V_{H}H, (b) a polypeptide linker, and (c) a SIRPα scFv. In some embodiments, a multivalent polypeptide of the disclosure include, in the N-terminal to C-terminal direction:

(a) a CD45 V_{H}H, (b) a polypeptide linker, and (c) a SIRPα scFv. In some embodiments, a multivalent polypeptide of the disclosure include, in the N-terminal to C-terminal direction:

(a) a SIRPα scFv, (b) a polypeptide linker, and (c) a CD45 V_{H}H.

In some embodiments, a multivalent polypeptide of the disclosure includes, in N-terminus to C-terminus direction: (i) a CD47 ECD, (ii) a GS linker, and (iii) a CD45 scFv. In some embodiments, a multivalent polypeptide of the disclosure includes, in N-terminus to C-terminus direction: (i) a CD47 ECD, (ii) a C3 linker, and (iii) a CD45 scFv.

In some embodiments, a multivalent polypeptide of the disclosure includes, in N-terminus to C-terminus direction: (i) a SIRPα scFv, (ii) a GS linker, and (iii) a CD45 scFv. In some embodiments, a multivalent polypeptide of the disclosure includes, in N-terminus to C-terminus direction: (i) a SIRPα scFv, (ii) a C3 linker, and (iii) a CD45 scFv.

In some embodiments, a multivalent polypeptide of the disclosure includes, in N-terminus to C-terminus direction: (i) a CD45 V_{H}H, (ii) a GS linker, and (iii) a SIRPα scFv. In some embodiments, a multivalent polypeptide of the disclosure includes, in N-terminus to C-terminus direction: (i) a CD45 V_{H}H, (ii) a C3 linker, and (iii) a SIRPα scFv.

In some embodiments, the CD47 ECD is a high affinity CD47 or a functional variant thereof. In some embodiments, the CD47 ECD is “Velcro.” In some embodiments, the polypeptide linker comprises a Gly-Ser (GS) linker. In some embodiments, the GS linker comprises the sequence of SEQ ID NO: 9. In some embodiments, the polypeptide linker comprises a 3C linker (LEVLFQGP; SEQ ID NO: 11).

In some embodiments, a multivalent polypeptide of the disclosure includes an amino acid sequence that has at least 80%, at least 85%, at least 90%, at least 95%, at least 96%, at least 97%, at least 98%, or at least 99% sequence identity to an amino acid sequence selected from the group consisting of SEQ ID NOS: 1-6. In some embodiments, a multivalent polypeptide of the disclosure includes an amino acid sequence that has at least 80%, at least 85%, at least 90%, at least 95%, at least 96%, at least 97%, at least 98%, or at least 99% sequence identity to the amino acid sequence of SEQ ID NO: 1. In some embodiments, a multivalent polypeptide of the disclosure includes an amino acid sequence that has at least 80%, at least 85%, at least 90%, at least 95%, at least 96%, at least 97%, at least 98%, or at least 99% sequence identity to the amino acid sequence of SEQ ID NO: 2. In some embodiments, a multivalent polypeptide of the disclosure includes an amino acid sequence that has at least 80%, at least 85%, at least 90%, at least 95%, at least 96%, at least 97%, at least 98%, or at least 99% sequence identity to the amino acid sequence of SEQ ID NO: 3. In some embodiments, a multivalent polypeptide of the disclosure includes an amino acid sequence that has at least 80%, at least 85%, at least 90%, at least 95%, at least 96%, at least 97%, at least 98%, or at least 99% sequence identity to the amino acid sequence of SEQ ID NO: 4. In some embodiments, a multivalent polypeptide of the disclosure includes an amino acid sequence that has at least 80%, at least 85%, at least 90%, at least 95%, at least 96%, at least 97%, at least 98%, or at least 99% sequence identity to the amino acid sequence of SEQ ID NO: 5. In some embodiments, a multivalent polypeptide of the disclosure includes an amino acid sequence that has at least 80%, at least 85%, at least 90%, at least 95%, at least 96%, at least 97%, at least 98%, or at least 99% sequence identity to the amino acid sequence of SEQ ID NO: 6.

In some embodiments, a multivalent polypeptide of the disclosure includes an amino acid sequence that has 100% sequence identity to an amino acid sequence selected from the group consisting of SEQ ID NOS: 1-6. In some embodiments, a multivalent polypeptide of the disclosure includes an amino acid sequence that has 100% sequence identity to the amino acid sequence of SEQ ID NO: 1. In some embodiments, a multivalent polypeptide of the disclosure includes an amino acid sequence that has 100% sequence identity to the amino acid sequence of SEQ ID NO: 2. In some embodiments, a multivalent polypeptide of the disclosure includes an amino acid sequence that has 100% sequence identity to the amino acid sequence of SEQ ID NO: 3. In some embodiments, a multivalent polypeptide of the disclosure includes an amino acid sequence that has 100% sequence identity to the amino acid sequence of SEQ ID NO: 4. In some embodiments, a multivalent polypeptide of the disclosure includes an amino acid sequence that has 100% sequence identity to the amino acid sequence of SEQ ID NO: 5. In some embodiments, a multivalent polypeptide of the disclosure includes an amino acid sequence that has 100% sequence identity to the amino acid sequence of SEQ ID NO: 6.

In some particular embodiments, the multivalent polypeptide of the present disclosure can be a multivalent antibody (e.g., bivalent antibody or trivalent antibody) including at least two antigen-binding moieties each possessing specific binding for a target protein. In some embodiments, the at least two antigen-binding moieties possess specific binding for the same target protein. Such antibody is multivalent, monospecific antibody. In some embodiments, the at least two antigen-binding moieties possessing specific binding for at least two different target proteins. Such antibody is multivalent, multispecific antibody (e.g., bispecific, trispecific, etc.) Accordingly, some embodiments disclosed herein relate to a multivalent antibody or functional fragment thereof, which includes (i) a first polypeptide module specific for one or more RPTPs, and (ii) a second polypeptide module specific for a SIRPα, wherein the first polypeptide module is operably linked to the second polypeptide module. Accordingly, in some embodiments, the disclosed multivalent antibody can be a bivalent, monospecific antibody. In some embodiments, the disclosed multivalent antibody can be a trivalent, monospecific antibody. In some embodiments, the disclosed multivalent antibody can be a bivalent, bispecific antibody. In some embodiments, the disclosed multivalent antibody can be a trivalent, trispecific antibody.

One skilled in the art will appreciate that the complete amino acid sequence can be used to construct a back-translated gene. For example, a DNA oligomer containing a nucleotide sequence coding for a given polypeptide can be synthesized. For example, several small oligonucleotides coding for portions of the desired polypeptide can be synthesized and then ligated. The individual oligonucleotides typically contain 5′ or 3′ overhangs for complementary assembly.

In addition to generating multivalent polypeptides via expression of nucleic acid molecules that have been altered by recombinant molecular biological techniques, a subject multivalent polypeptide or multivalent antibody in accordance with the present disclosure can be chemically synthesized. Chemically synthesized polypeptides are routinely generated by those of skill in the art.

Once assembled (by synthesis, site-directed mutagenesis or another method), the DNA sequences encoding a multivalent polypeptide or multivalent antibody as disclosed herein will be inserted into an expression vector and operably linked to an expression control sequence appropriate for expression of the multivalent polypeptide or multivalent antibody in the desired transformed host. Proper assembly can be confirmed by nucleotide sequencing, restriction mapping, and expression of a biologically active polypeptide in a suitable host. As is known in the art, in order to obtain high expression levels of a transfected gene in a host, the gene must be operably linked to transcriptional and translational expression control sequences that are functional in the chosen expression host.

The binding activity of the multivalent polypeptides and multivalent antibodies of the disclosure can be assayed by any suitable method known in the art. For example, the binding activity of the multivalent polypeptides and multivalent antibodies of the disclosure can be determined by, e.g., Scatchard analysis (Munsen, et al. 1980 Analyt. Biochem. 107:220-239). Specific binding may be assessed using techniques known in the art including but not limited to competition ELISA, BIACORE® assays and/or KINEXA® assays. An antibody or polypeptide that “preferentially binds” or “specifically binds” (used interchangeably herein) to a target protein or target epitope is a term well understood in the art, and methods to determine such specific or preferential binding are also known in the art. An antibody or polypeptide is said to exhibit “specific binding” or “preferential binding” if it reacts or associates more frequently, more rapidly, with greater duration and/or with greater affinity with a particular protein or epitope than it does with alternative proteins or epitopes. An antibody or polypeptide “specifically binds” or “preferentially binds” to a target if it binds with greater affinity, avidity, more readily, and/or with greater duration than it binds to other substances. Also, an antibody or polypeptide “specifically binds” or “preferentially binds” to a target if it binds with greater affinity, avidity, more readily, and/or with greater duration to that target in a sample than it binds to other substances present in the sample. For example, an antibody or polypeptide that specifically or preferentially binds to a SIRPα epitope is an antibody or polypeptide that binds this epitope with greater affinity, avidity, more readily, and/or with greater duration than it binds to other SIRPα epitopes or non-SIRPα epitopes. It is also understood by reading this definition, for example, that an antibody or polypeptide (or moiety or epitope) which specifically or preferentially binds to a first target may or may not specifically or preferentially bind to a second target. As such, “specific binding” or “preferential binding” does not necessarily require (although it can include) exclusive binding.

A variety of assay formats may be used to select an antibody or polypeptide that specifically binds a molecule of interest. For example, solid-phase ELISA immunoassay, immunoprecipitation, Biacore® (GE Healthcare, Piscataway, NJ), KinExA, fluorescence-activated cell sorting (FACS), Octet® (ForteBio, Inc., Menlo Park, CA) and Western blot analysis are among many assays that may be used to identify an antibody that specifically reacts with an antigen or a receptor, or ligand binding portion thereof, that specifically binds with a cognate ligand or binding partner. Generally, a specific or selective reaction will be at least twice the background signal or noise, more typically more than 10 times background, even more typically, more than 50 times background, more typically, more than 100 times background, yet more typically, more than 500 times background, even more typically, more than 1000 times background, and even more typically, more than 10,000 times background. Also, an antibody is said to “specifically bind” an antigen when the equilibrium dissociation constant (K_{D}) is < 7 nM.

The term “binding affinity” is herein used as a measure of the strength of a noncovalent interaction between two molecules, e.g., an antibody or portion thereof and an antigen. The term “binding affinity” is used to describe monovalent interactions (intrinsic activity). Binding affinity between two molecules may be quantified by determination of the dissociation constant (K_{D}). In tum, K_{D} can be determined by measurement of the kinetics of complex formation and dissociation using, e.g., the surface plasmon resonance (SPR) method (Biacore). The rate constants corresponding to the association and the dissociation of a monovalent complex are referred to as the association rate constants kₐ (or kₒₙ) and dissociation rate constant k_{d} (or k_{off}), respectively. K_{D} is related to kₐ and k_{d} through the equation K_{D} = kₐ / kₐ. The value of the dissociation constant can be determined directly by well-known methods, and can be computed even for complex mixtures by methods such as those set forth in Caceci et al. (1984, Byte 9: 340-362). For example, the K_{D} may be established using a double-filter nitrocellulose filter binding assay such as that disclosed by Wong & Lohman (1993, Proc. Natl. Acad. Sci. USA 90: 5428- 5432). Other standard assays to evaluate the binding ability of antibodies or polypeptides of the present disclosure towards target antigens are known in the art, including for example, ELISAs, Western blots, RIAs, and flow cytometry analysis, and other assays exemplified elsewhere herein. The binding kinetics and binding affinity of the antibody also can be assessed by standard assays known in the art, such as Surface Plasmon Resonance (SPR), e.g., by using a Biacore® system, or KinExA.

### Nucleic Acid Molecules

In one aspect, some embodiments disclosed herein relate to recombinant nucleic acid molecules encoding the multivalent polypeptides and multivalent antibodies of the disclosure, expression cassettes, and expression vectors containing these nucleic acid molecules operably linked to regulator sequences which allow expression of the multivalent polypeptides and multivalent antibodies in a host cell or ex-vivo cell-free expression system.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR §1.822 is used herein.

Nucleic acid molecules of the present disclosure can be nucleic acid molecules of any length, including nucleic acid molecules that are generally between about generally between about 0.5 Kb and about 20 Kb, for example between about 0.5 Kb and about 20 Kb, between about 1 Kb and about 15 Kb, between about 2 Kb and about 10 Kb, or between about 5 Kb and about 25 Kb, for example between about 10 Kb to 15 Kb, between about 15 Kb and about 20 Kb, between about 5 Kb and about 20 Kb, about 5 Kb and about 10 Kb, or about 10 Kb and about 25 Kb.

In some embodiments disclosed herein, the nucleic acid molecules of the disclosure include a nucleotide sequence encoding a multivalent polypeptide which include (i) a first amino acid sequence including a first polypeptide module capable of binding to a RPTP, and (ii) a second amino acid sequence including a second polypeptide module capable of binding to one or more SIRPα molecules that signal through a phosphorylation mechanism, wherein the first polypeptide module is operably linked to the second polypeptide module. In some embodiments, the nucleic acid molecules of the disclosure include a nucleotide sequence encoding a multivalent antibody which includes a (i) a first polypeptide module specific for one or more RPTPs, and (ii) a second polypeptide module specific for one or more SIRPα molecules that signal through a phosphorylation mechanism.

In some embodiments disclosed herein, the nucleic acid molecules include a nucleotide sequence encoding a polypeptide that includes (i) an amino acid sequence having at least 80% sequence identity to the amino acid sequence of a multivalent polypeptide as disclosed herein or a functional fragment thereof; or (ii) an amino acid sequence having at least 80% sequence identity to the multivalent antibody of or a functional fragment thereof as disclosed herein. The nucleic acid molecules include a nucleotide sequence encoding a polypeptide that includes (i) an amino acid sequence having at least 90%, at least 95%, at least 96%, at least 97%, at least 98%, at least 99%, or 100% sequence identity to the amino acid sequence of a multivalent polypeptide as disclosed herein or a functional fragment thereof; or (ii) an amino acid sequence having at least 90%, at least 95%, at least 96%, at least 97%, at least 98%, at least 99%, or 100% sequence identity to the multivalent antibody of or a functional fragment thereof as disclosed herein.

In some embodiments, the nucleic acid molecules include a nucleotide sequence encoding an amino acid sequence that has at least 80%, at least 85%, at least 90%, at least 95%, at least 96%, at least 97%, at least 98%, at least 99%, or 100% sequence identity to an amino acid sequence selected from the group consisting of SEQ ID NOS: 1-6 or a functional fragment thereof. In some embodiments, the nucleic acid molecules include a nucleotide sequence encoding an amino acid sequence that has at least 80%, at least 85%, at least 90%, at least 95%, at least 96%, at least 97%, at least 98%, at least 99%, or 100% sequence identity to SEQ ID NO: 1, or a functional fragment thereof. In some embodiments, the nucleic acid molecules include a nucleotide sequence encoding an amino acid sequence that has at least 80%, at least 85%, at least 90%, at least 95%, at least 96%, at least 97%, at least 98%, at least 99%, or 100% sequence identity to SEQ ID NO: 2, or a functional fragment thereof. In some embodiments, the nucleic acid molecules include a nucleotide sequence encoding an amino acid sequence that has at least 80%, at least 85%, at least 90%, at least 95%, at least 96%, at least 97%, at least 98%, at least 99%, or 100% sequence identity to SEQ ID NO: 3, or a functional fragment thereof. In some embodiments, the nucleic acid molecules include a nucleotide sequence encoding an amino acid sequence that has at least 80%, at least 85%, at least 90%, at least 95%, at least 96%, at least 97%, at least 98%, at least 99%, or 100% sequence identity to SEQ ID NO: 4, or a functional fragment thereof. In some embodiments, the nucleic acid molecules include a nucleotide sequence encoding an amino acid sequence that has at least 80%, at least 85%, at least 90%, at least 95%, at least 96%, at least 97%, at least 98%, at least 99%, or 100% sequence identity to SEQ ID NO: 5, or a functional fragment thereof. In some embodiments, the nucleic acid molecules include a nucleotide sequence encoding an amino acid sequence that has at least 80%, at least 85%, at least 90%, at least 95%, at least 96%, at least 97%, at least 98%, at least 99%, or 100% sequence identity to SEQ ID NO: 6, or a functional fragment thereof.

In some embodiments, the recombinant nucleic acid molecules as disclosed herein can be incorporated into an expression cassette or an expression vector. Accordingly, some embodiments disclosed herein relate to vectors or expression cassettes including a recombinant nucleic acid molecule as disclosed herein. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure include a coding sequence for a multivalent polypeptide as disclosed herein, which is operably linked to expression control elements, such as a promoter, and optionally, any or a combination of other nucleic acid sequences that affect the transcription or translation of the coding sequence.

In some embodiments, the nucleic acid molecules of the disclosure can be incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector. Accordingly, also provided herein are vectors, plasmids or viruses containing one or more of the nucleic acid molecules encoding any of the multivalent polypeptides and multivalent antibodies disclosed herein. The nucleic acid molecules described above can be contained within a vector that is capable of directing their expression in, for example, a cell that has been transduced with the vector. Suitable vectors for use in eukaryotic and prokaryotic cells are known in the art and are commercially available or readily prepared by a skilled artisan. Additional vectors can also be found, for example, in Ausubel, F. M., et al., Current Protocols in Molecular Biology, (Current Protocol, 1994) and Sambrook et al., “Molecular Cloning: A Laboratory Manual,” 2nd ED. (1989).

It should be understood that not all vectors and expression control sequences will function equally well to express the DNA sequences described herein. Neither will all hosts function equally well with the same expression system. However, one of skill in the art may make a selection among these vectors, expression control sequences and hosts without undue experimentation. For example, in selecting a vector, the host must be considered because the vector must replicate in it. The vector’s copy number, the ability to control that copy number, and the expression of any other proteins encoded by the vector, such as antibiotic markers, should also be considered. For example, vectors that can be used include those that allow the DNA encoding the multivalent polypeptides and multivalent antibodies of the present disclosure to be amplified in copy number. Such amplifiable vectors are known in the art. They include, for example, vectors able to be amplified by DHFR amplification (see, e.g., Kaufman, U.S. Pat. No. 4,470,461) or glutamine synthetase (“GS”) amplification (see, e.g., U.S. Pat. No. 5,122,464 and European published application EP 338,841).

Accordingly, in some embodiments, the multivalent polypeptides and multivalent antibodies of the present disclosure can be expressed from vectors, generally expression vectors. The vectors are useful for autonomous replication in a host cell or may be integrated into the genome of a host cell upon introduction into the host cell, and thereby are replicated along with the host genome (e.g., non-episomal mammalian vectors). Expression vectors are capable of directing the expression of coding sequences to which they are operably linked. In general, expression vectors of utility in recombinant DNA techniques are often in the form of plasmids (vectors). However, other forms of expression vectors, such as viral vectors (e.g., replication defective retroviruses, adenoviruses, and adeno-associated viruses) are also included.

Exemplary recombinant expression vectors can include one or more regulatory sequences, selected on the basis of the host cells to be used for expression, operably linked to the nucleic acid sequence to be expressed.

DNA vector can be introduced into prokaryotic or eukaryotic cells via conventional transformation or transfection techniques. Suitable methods for transforming or transfecting host cells can be found in Sambrook et al. (1989) Molecular Cloning: A Laboratory Manual (2nd ed., Cold Spring Harbor Laboratory Press, Plainview, N.Y.) and other standard molecular biology laboratory manuals.

The nucleic acid sequences encoding the multivalent polypeptides and multivalent antibodies of the present disclosure can be optimized for expression in the host cell of interest. For example, the G-C content of the sequence can be adjusted to levels average for a given cellular host, as calculated by reference to known genes expressed in the host cell. Methods for codon optimization are known in the art. Codon usages within the coding sequence of the multivalent polypeptides and multivalent antibodies disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Vectors suitable for use include T7-based vectors for use in bacteria, the pMSXND expression vector for use in mammalian cells, and baculovirus-derived vectors for use in insect cells. In some embodiments nucleic acid inserts, which encode the subject multivalent polypeptide or multivalent antibody in such vectors, can be operably linked to a promoter, which is selected based on, for example, the cell type in which expression is sought.

In selecting an expression control sequence, a variety of factors should also be considered. These include, for example, the relative strength of the sequence, its controllability, and its compatibility with the actual DNA sequence encoding the subject multivalent polypeptide or multivalent antibody, particularly as regards potential secondary structures. Hosts should be selected by consideration of their compatibility with the chosen vector, the toxicity of the product coded for by the DNA sequences of this disclosure, their secretion characteristics, their ability to fold the polypeptides correctly, their fermentation or culture requirements, and the ease of purification of the products coded for by the DNA sequences.

Within these parameters one of skill in the art may select various vector/expression control sequence/host combinations that will express the desired DNA sequences on fermentation or in large scale animal culture, for example, using CHO cells or COS 7 cells.

The choice of expression control sequence and expression vector, in some embodiments, will depend upon the choice of host. A wide variety of expression host/vector combinations can be employed. Non-limiting examples of useful expression vectors for eukaryotic hosts, include, for example, vectors with expression control sequences from SV40, bovine papilloma virus, adenovirus and cytomegalovirus. Non-limiting examples of useful expression vectors for bacterial hosts include known bacterial plasmids, such as plasmids from E. coli, including col El, pCRI, pER32z, pMB9 and their derivatives, wider host range plasmids, such as RP4, phage DNAs, e.g., the numerous derivatives of phage lambda, e.g., NM989, and other DNA phages, such as M13 and filamentous single stranded DNA phages. Non-limiting examples of useful expression vectors for yeast cells include the 2µ plasmid and derivatives thereof. Non-limiting examples of useful vectors for insect cells include pVL 941 and pFastBac® 1.

In addition, any of a wide variety of expression control sequences can be used in these vectors. Such useful expression control sequences include the expression control sequences associated with structural genes of the foregoing expression vectors. Examples of useful expression control sequences include, for example, the early and late promoters of SV40 or adenovirus, the lac system, the trp system, the TAC or TRC system, the major operator and promoter regions of phage lambda, for example PL, the control regions of fd coat protein, the promoter for 3-phosphoglycerate kinase or other glycolytic enzymes, the promoters of acid phosphatase, e.g., PhoA, the promoters of the yeast a-mating system, the polyhedron promoter of Baculovirus, and other sequences known to control the expression of genes of prokaryotic or eukaryotic cells or their viruses, and various combinations thereof.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans will readily appreciate numerous promoters and other regulatory elements which can be used to direct expression of nucleic acids.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed, and thus permits phenotypic selection of the transfected cells. Those of skill in the art can readily determine whether a given regulatory element or selectable marker is suitable for use in a particular experimental context.

Viral vectors that can be used in the disclosure include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

In selecting an expression system, care should be taken to ensure that the components are compatible with one another. For example, an multivalent polypeptide or multivalent antibody as disclosed herein can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed multivalent polypeptides or multivalent antibodies can be purified from the expression system using routine biochemical procedures, and can be used, e.g., as therapeutic agents, as described herein.

In some embodiments, multivalent polypeptides or multivalent antibodies obtained will be glycosylated or unglycosylated depending on the host organism used to produce the multivalent polypeptides or multivalent antibodies. If bacteria are chosen as the host then the multivalent polypeptide or multivalent antibody produced will be unglycosylated. Eukaryotic cells, on the other hand, will glycosylate the multivalent polypeptides or multivalent antibodies, although perhaps not in the same way as native polypeptides is glycosylated. The multivalent polypeptides or multivalent antibodies produced by the transformed host can be purified according to any suitable methods known in the art. Produced multivalent polypeptides or multivalent antibodies can be isolated from inclusion bodies generated in bacteria such as E. coli, or from conditioned medium from either mammalian or yeast cultures producing a given multivalent polypeptide or multivalent antibody using cation exchange, gel filtration, and or reverse phase liquid chromatography.

In addition or alternatively, another exemplary method of constructing a DNA sequence encoding the multivalent polypeptides or multivalent antibodies of the disclosure is by chemical synthesis. This includes direct synthesis of a peptide by chemical means of the protein sequence encoding for a multivalent polypeptide or multivalent antibody exhibiting the properties described. This method can incorporate both natural and unnatural amino acids at positions that affect the binding affinity of the multivalent polypeptide or multivalent antibody with the target protein. Alternatively, a gene which encodes the desired multivalent polypeptide or multivalent antibody can be synthesized by chemical means using an oligonucleotide synthesizer. Such oligonucleotides are designed based on the amino acid sequence of the desired multivalent polypeptide or multivalent antibody, and generally selecting those codons that are favored in the host cell in which the recombinant multivalent polypeptide or multivalent antibody will be produced. In this regard, it is well recognized in the art that the genetic code is degenerate-that an amino acid may be coded for by more than one codon. For example, Phe (F) is coded for by two codons, TIC or TTT, Tyr (Y) is coded for by TAC or TAT and his (H) is coded for by CAC or CAT. Trp (W) is coded for by a single codon, TGG. Accordingly, it will be appreciated by those skilled in the art that for a given DNA sequence encoding a particular multivalent polypeptide or multivalent antibody, there will be many DNA degenerate sequences that will code for that multivalent polypeptide or multivalent antibody. For example, it will be appreciated that in addition to the DNA sequences for multivalent polypeptides or multivalent antibodies provided in the Sequence Listing, there will be many degenerate DNA sequences that code for the multivalent polypeptides or multivalent antibodies disclosed herein. These degenerate DNA sequences are considered within the scope of this disclosure. Therefore, “degenerate variants thereof” in the context of this disclosure means all DNA sequences that code for and thereby enable expression of a particular multivalent polypeptide or multivalent antibody.

The DNA sequence encoding the subject multivalent polypeptide or multivalent antibody, whether prepared by site directed mutagenesis, chemical synthesis or other methods, can also include DNA sequences that encode a signal sequence. Such signal sequence, if present, should be one recognized by the cell chosen for expression of the multivalent polypeptide or multivalent antibody. It can be prokaryotic, eukaryotic or a combination of the two. In general, the inclusion of a signal sequence depends on whether it is desired to secrete the multivalent polypeptide or multivalent antibody as disclosed herein from the recombinant cells in which it is made. If the chosen cells are prokaryotic, the DNA sequence generally does not encode a signal sequence. If the chosen cells are eukaryotic, a signal sequence is generally included.

The nucleic acid molecules provided can contain naturally occurring sequences, or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides; some or all of the non-coding sequences that lie upstream or downstream from a coding sequence (e.g., the coding sequence of SIRPα or a RIPR-SIRPα molecule of the disclosure) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

Exemplary nucleic acid molecules of the present disclosure can include fragments not found as such in the natural state. Thus, this disclosure encompasses recombinant nucleic acid molecules, such as those in which a nucleic acid sequence (for example, a sequence encoding a RIPR-SIRPα molecule of the disclosure) is incorporated into a vector (e.g., a plasmid or viral vector) or into the genome of a heterologous cell (or the genome of a homologous cell, at a position other than the natural chromosomal location).

### Recombinant Cells and Cell Cultures

The multivalent polypeptides and recombinant nucleic acids of the present disclosure can be introduced into a cell, such as, for example, a human phagocytic cell, to produce a recombinant cell, e.g., an engineered cell. In some embodiments, the cell is in vivo. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell.

For example, a multivalent polypeptide and/or recombinant nucleic acid as disclosed herein can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). In some embodiments, the recombinant cell is a phagocytic cell, e.g., phagocyte. Both professional phagocytes and non-professional phagocytes are suitable. In some embodiments, the phagocytic cell is a professional phagocyte. In some embodiments, the phagocytic cell is a non-professional phagocyte. In some embodiments, the phagocytic cell is selected from the group consisting of macrophages, dendritic cells, mast cells, monocytes, neutrophils, microglia, and astrocytes. In some embodiments, the phagocytic cell is a dendritic cell. In some embodiments, the phagocytic cell is a bone marrow-derived macrophage (BMDM) or a bone marrow-derived dendritic cell (BMDC). These cells are available from many sources, including the American Type Culture Collection (Manassas, Va.).

Accordingly, some embodiments of the disclosure relate to methods for making a recombinant cell, including (a) providing a host cell capable of protein expression; and transducing the provided host cell with a recombinant nucleic acid molecule of the disclosure to produce a recombinant cell. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be introduced into a host cell by viral or non-viral delivery vehicles known in the art to produce a recombinant cell. For example, the nucleic acid molecule can be stably integrated in the recombinant cell’s genome, or can be episomally replicating, or present in the recombinant cell as a mini-circle expression vector for transient expression. Accordingly, in some embodiments, the nucleic acid molecule is maintained and replicated in the recombinant host cell as an episomal unit. In some embodiments, the nucleic acid molecule is present in the recombinant cell as a mini-circle expression vector for transient expression. In some embodiments, the nucleic acid molecule is stably integrated into the genome of the recombinant cell. Stable integration can be achieved using classical random genomic recombination techniques or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases).

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, baculoviral virus or adeno-associated virus (AAV) can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present disclosure that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In another aspect, provided herein are cell cultures including at least one recombinant cell as disclosed herein, and a culture medium. Generally, the culture medium can be any suitable culture medium for culturing the cells described herein. As discusses above, techniques for transforming a wide variety of the above-mentioned cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Methods For Promoting Maturation of Immature Dendritic Cells (DCs)

As discussed in greater detail below, some embodiments of the disclosure relate to methods for promoting the maturation of immature dendritic cells (DCs) in vitro. DCs are specialized for presenting antigens to naive or quiescent T cells. Consequently, DCs play a central role in modulating immunity in vivo. Immunization using DCs loaded with selected antigens represents a powerful method of inducing immunity against pathogens or tumors. Under appropriate conditions, DCs can also tolerize T cells and hence suppress an immune response against specific antigens.

The ability of DCs to induce an immune response requires antigen uptake, which occurs principally in non-lymphoid organs, followed by antigen presentation and activation of T cells in the lymph system. These separate functional roles are performed by immature DCs and mature DCs, respectively.

DCs are among the most powerful antigen-presenting cells for priming both CD8⁺ cytotoxic T-cells (CTL) and CD4+ T-helper (Th1) responses. They are capable of capturing and processing antigens and migrating to the regional lymph nodes to induce CD8⁺ T-cell responses. They have the capacity to cross-present exogenous antigens in the context of MHC class I molecules present on the cell surface. These features taken together enable the dendritic cells to present antigen in a manner which is capable of priming both CD8⁺ and CD4⁺ T-cell responses, providing a rationale for the use of DCs as a cellular vaccine.

In some embodiments, provided herein are methods for promoting the maturation of immature dendritic cells (DCs) in vitro, the methods include: (a) exposing immature DCs to an antigen; and (b) culturing the immature DCs in the presence of a multivalent polypeptide of the disclosure to induce the maturation of immature DCs into mature DCs.

The immature DCs may be exposed to the antigen for sufficient time to induce the dendritic cells to capture and process the antigen. In some embodiments, the mature DCs have elevated CD86 expression levels compared to a reference mature DC cultured in the absence of the multivalent polypeptide of the disclosure. In some embodiments, the immature DCs are cultured from peripheral blood mononuclear cells isolated from a mammal, such as a mouse, a human, or a non-human primate. In some embodiments, the exposing of immature DCs to an antigen produce antigen-presenting immature DCs. In some embodiments, the culturing the produced antigen-presenting immature DCs in the presence of a multivalent polypeptide of the disclosure promotes the maturation of the antigen-presenting immature DCs to produce mature antigen-presenting DCs.

The antigen can generally be any antigen, for example, a cancer antigen, e.g., a tumor-associated antigen. The antigen can alternatively be an antigen derived from a human parasite, virus or microorganism. Accordingly, in some embodiments, the methods include exposing immature DCs to an antigen wherein the antigen is selected from the group consisting of human parasite antigens, animal parasite antigens, human virus antigens, animal virus antigens, human microorganism antigens, and microorganism antigens. In some embodiments, the antigen is a cancer-associated antigen. In some embodiments, the antigen is a cancer-specific antigen. Accordingly, mature DCs prepared by a method of the disclosure are also within the scope of this disclosure.

In another aspect, provided are methods for manufacturing a vaccine, the methods include: (a) exposing immature DCs to an antigen in vitro to produce a sufficient number of antigen-presenting immature DCs; and (b) promoting the maturation of the antigen-presenting immature DCs in the presence of a multivalent polypeptide of the disclosure to produce mature antigen-presenting DCs. In some embodiments, the antigen is selected from the group consisting of human parasite antigens, animal parasite antigens, human virus antigens, animal virus antigens, human microorganism antigens, and animal microorganism antigens. In some embodiments, the antigen is a cancer-associated antigen. In some embodiments, the antigen is a cancer-specific antigen. The mature antigen-presenting DCs produced as described herein are suitable for use in a vaccine. For example, a vaccine for stimulating the cellular immune response in a subject diagnosed with health condition, e.g., cancer, can be produced. In this exemplary method of producing the vaccine, immature DCs are exposed to the subject’s tumor-associated antigens to produce tumor antigen presenting immature DCs. The antigen presenting dendritic cells are then matured in the presence of a multivalent polypeptide according to a method described herein, and then included in a pharmaceutical formulation in the form of a vaccine. The vaccine can then be injected into the subject, whereupon it is expected that the mature DCs will migrate to the subject’s regional lymph nodes to induce CTL (cytotoxic CD8+ T-cell lymphocytes) response.

Accordingly, vaccines manufactured by a method of manufacturing vaccines as disclosed herein are also within the scope of the present disclosure. In some embodiments, the vaccines may be for use in a method of preventing or treating a subject with a health condition such as a proliferative disease (e.g., cancer) or diagnosed with a microbial infection (e.g., virus, micro-fungus, or bacterium) or a parasitic infection. In some embodiments, the vaccines of the disclosure further include one or more suitable a diluent, an excipient or auxiliary. In some embodiments, the vaccines of the disclosure further include one or more suitable bacterial adjuvant, and a systemic adjuvant. In some embodiments, the vaccines of the disclosure further include one or more of the following: a diluent, an excipient, an auxiliary adjuvant, a bacterial adjuvant, and a systemic adjuvant.

### Compositions and Pharmaceutical Compositions

In some embodiments, the multivalent polypeptides, multivalent antibodies, nucleic acids, mature DCs, and vaccines of the present disclosure can be incorporated into compositions, including pharmaceutical compositions. Such compositions generally include one or more the multivalent polypeptides, multivalent antibodies, nucleic acids, mature DCs, and vaccines of the disclosure. In some embodiments, the compositions are pharmaceutical compositions. In some embodiments, the pharmaceutical compositions of the disclosure include a pharmaceutically acceptable excipient and one or more the following: multivalent polypeptides, multivalent antibodies, nucleic acids, mature DCs, and vaccines of the disclosure.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL®. (BASF, Parsippany, N.J.) or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the common methods of preparation are vacuum drying and freeze-drying which yields a powder of the active ingredient plus any additional desired ingredient from a previously sterile-filtered solution thereof.

Oral compositions, if used, generally include an inert diluent or an edible carrier. For the purpose of oral therapeutic administration, the active compound (e.g., multivalent polypeptides, multivalent antibodies, nucleic acid molecules, and/or vaccines of the disclosure) can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel®, or com starch; a lubricant such as magnesium stearate or Sterotes®; a glidant such as colloidal silicon dioxide; a sweetening agent such as sucrose or saccharin; or a flavoring agent such as peppermint, methyl salicylate, or orange flavoring.

In the event of administration by inhalation, the subject multivalent polypeptides, multivalent antibodies, nucleic acids, mature DCs, and vaccines of the disclosure are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration of the subject multivalent polypeptides, multivalent antibodies, nucleic acids, mature DCs, and vaccines of the disclosure can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, the multivalent polypeptides, multivalent antibodies, nucleic acids, mature DCs, and vaccines of the disclosure can also be prepared in the form of suppositories (e.g., with conventional suppository bases such as cocoa butter and other glycerides) or retention enemas for rectal delivery.

In some embodiments, the multivalent polypeptides, multivalent antibodies, nucleic acids, mature DCs, and vaccines of the disclosure can also be administered by transfection or infection using methods known in the art, including but not limited to the methods described in McCaffrey et al. (Nature 418:6893, 2002), Xia et al. (Nature Biotechnol. 20: 1006-1010, 2002), or Putnam (Am. J. Health Syst. Pharm. 53: 151-160, 1996, erratum at Am. J. Health Syst. Pharm. 53:325, 1996).

In some embodiments, the subject multivalent polypeptides, multivalent antibodies, nucleic acids, mature DCs, and vaccines of the disclosure are prepared with carriers that will protect the multivalent polypeptides, multivalent antibodies, nucleic acids, mature DCs, and vaccines against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

As described in greater detail below, the multivalent polypeptides and multivalent antibodies of the present disclosure may also be modified to achieve extended duration of action such as by PEGylation, acylation, Fc fusions, linkage to molecules such as albumin, etc. In some embodiments, the multivalent polypeptides or multivalent antibodies can be further modified to prolong their half-life in vivo and/or ex vivo. Non-limiting examples of known strategies and methodologies suitable for modifying the multivalent polypeptides or multivalent antibodies of the disclosure include (1) chemical modification of a multivalent polypeptide or multivalent antibody described herein with highly soluble macromolecules such as polyethylene glycol (PEG) which prevents the multivalent polypeptide or multivalent antibody from contacting with proteases; and (2) covalently linking or conjugating a multivalent polypeptide or multivalent antibody described herein with a stable protein such as, for example, albumin. Accordingly, in some embodiments, the multivalent polypeptide or multivalent antibody of the disclosure can be fused to a stable protein, such as, albumin. For example, human albumin is known as one of the most effective proteins for enhancing the stability of polypeptides fused thereto and there are many such fusion proteins reported.

In some embodiments, the pharmaceutical compositions of the disclosure include one or more PEGylation reagents. As used herein, the term “PEGylation” refers to modifying a protein by covalently attaching polyethylene glycol (PEG) to the protein, with “PEGylated” referring to a protein having a PEG attached. A range of PEG, or PEG derivative sizes with optional ranges of from about 10,000 Daltons to about 40,000 Daltons may be attached to the multivalent polypeptides or multivalent antibodies of the disclosure using a variety of chemistries. In some embodiments, the PEGylation reagent is selected from methoxy polyethylene glycol-succinimidyl propionate (mPEG-SPA), mPEG-succinimidyl butyrate (mPEG-SBA), mPEG-succinimidyl succinate (mPEG-SS), mPEG-succinimidyl carbonate (mPEG-SC), mPEG-Succinimidyl Glutarate (mPEG-SG), mPEG-N-hydroxyl-succinimide (mPEG-NHS), mPEG-tresylate and mPEG-aldehyde. In some embodiments, the PEGylation reagent is polyethylene glycol; for example said pegylation reagent is polyethylene glycol with an average molecular weight of 20,000 Daltons covalently bound to the N-terminal methionine residue of the multivalent polypeptides and multivalent antibodies of the disclosure.

Accordingly, in some embodiments, the multivalent polypeptides and multivalent antibodies of the disclosure are chemically modified with one or more polyethylene glycol moieties, e.g., PEGylated; or with similar modifications, e.g. PASylated. In some embodiments, the PEG molecule or PAS molecule is conjugated to one or more amino acid side chains of the multivalent polypeptide or multivalent antibody. In some embodiments, the PEGylated or PASylated multivalent polypeptide or multivalent antibody contains a PEG or PAS moiety on only one amino acid. In other embodiments, the PEGylated or PASylated multivalent polypeptide or multivalent antibody contains a PEG or PAS moiety on two or more amino acids, e.g., attached to two or more, five or more, ten or more, fifteen or more, or twenty or more different amino acid residues. In some embodiments, the PEG or PAS chain is 2000, greater than 2000, 5000, greater than 5,000, 10,000, greater than 10,000, greater than 10,000, 20,000, greater than 20,000, and 30,000 Da. The PASylated multivalent polypeptide or multivalent antibody may be coupled directly to PEG or PAS (e.g., without a linking group) through an amino group, a sulfhydryl group, a hydroxyl group, or a carboxyl group. In some embodiments, the multivalent polypeptide or multivalent antibody of the disclosure is covalently bound to a polyethylene glycol with an average molecular weight of 20,000 Daltons.

In some embodiments, the multivalent polypeptides or multivalent antibodies of the disclosure can be further modified to prolong their half-life in vivo and/or ex vivo. Non-limiting examples of known strategies and methodologies suitable for modifying the multivalent polypeptides or multivalent antibodies of the disclosure include (1) chemical modification of a multivalent polypeptide or multivalent antibody described herein with highly soluble macromolecules such as polyethylene glycol (“PEG”) which prevents the multivalent polypeptide or multivalent antibody from contacting with proteases; and (2) covalently linking or conjugating a multivalent polypeptide or multivalent antibody described herein with a stable protein such as, for example, albumin. Accordingly, in some embodiments, the multivalent polypeptide or multivalent antibody of the disclosure can be fused to a stable protein, such as, albumin. For example, human albumin is known as one of the most effective proteins for enhancing the stability of polypeptides fused thereto and there are many such fusion proteins reported.

### Methods of the Disclosure

Administration of any one of the therapeutic compositions described herein, e.g., multivalent polypeptides, multivalent antibodies, nucleic acids, recombinant cells, cell cultures, vaccines, and pharmaceutical compositions, can be used in the prevention or treatment of relevant health conditions, such as proliferative diseases (e.g., cancers), autoimmune diseases, and microbial infections (e.g., bacterial infections or viral infections). In some embodiments, the infection is chronic infection. In some embodiments, the multivalent polypeptides, multivalent antibodies, nucleic acids, recombinant cells, cell cultures, vaccines, and/or pharmaceutical compositions as described herein can be incorporated into therapeutic agents for use in methods of treating an individual who has, who is suspected of having, or who may be at high risk for developing one or more health conditions or diseases associated with cell signaling mediated by CD47 and/or SIRPα. Exemplary health conditions or diseases can include, without limitation, cancers and chronic infection. In some embodiments, the individual is a patient under the care of a physician.

Accordingly, in one aspect, some embodiments of the disclosure relate to methods for modulating cell signaling mediated by CD47 and/or SIRPα, the method includes administering to the subject a composition including one or more of: (i) a multivalent polypeptide of the disclosure, (ii) a multivalent antibody of the disclosure, (iii) a recombinant nucleic acid molecule of the disclosure, (iv) a recombinant cell of the disclosure, (v) a mature DC of the disclosure; and (vi) a vaccine of the disclosure. In another aspect, some embodiments of the disclosure relate to methods for the prevention or treatment of a health condition in a subject in need thereof, the method including administering to the subject a composition including one or more of: (i) a multivalent polypeptide of the disclosure, (ii) a multivalent antibody of the disclosure, (iii) a recombinant nucleic acid molecule of the disclosure, (iv) a recombinant cell of the disclosure, (v) a mature DC of the disclosure; and (vi) a vaccine of the disclosure. In some embodiments, the methods include administering a therapeutically effective amount of (i) a multivalent polypeptide of the disclosure, (ii) a multivalent antibody of the disclosure, (iii) a recombinant nucleic acid molecule of the disclosure, (iv) a recombinant cell of the disclosure, (v) a mature DC of the disclosure; and (vi) a vaccine of the disclosure.

Non-limiting exemplary embodiments of the methods of prevention or treating a health condition described herein can include one or more of the following features. In some embodiments, the health condition is a proliferative disease or an infection. Exemplary proliferative diseases can include, without limitation, angiogenic diseases, a metastatic diseases, tumorigenic diseases, neoplastic diseases and cancers. In some embodiments, the proliferative disease is a cancer. In some embodiments, the cancer is a pediatric cancer. In some embodiments, the cancer is a pancreatic cancer, a colon cancer, an ovarian cancer, a prostate cancer, a lung cancer, mesothelioma, a breast cancer, a urothelial cancer, a liver cancer, a head and neck cancer, a sarcoma, a cervical cancer, a stomach cancer, a gastric cancer, a melanoma, a uveal melanoma, a cholangiocarcinoma, multiple myeloma, leukemia, lymphoma, and glioblastoma.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the subject inhibits metastasis of the cancer in the subject. In some embodiments, the administered composition inhibits tumor growth in the subject.

Exemplary proliferative diseases can include, without limitation, angiogenic diseases, a metastatic diseases, tumorigenic diseases, neoplastic diseases and cancers. In some embodiments, the proliferative disease is a cancer. The term “cancer” generally refers to a disease characterized by the rapid and uncontrolled growth of aberrant cells. The aberrant cells may form solid tumors or constitute a hematological malignancy. Cancer cells can spread locally or through the bloodstream and lymphatic system to other parts of the body. There are no specific limitations with respect to the cancers which can be treated by the compositions and methods of the present disclosure. Non-limiting examples of suitable cancers include ovarian cancer, renal cancer, breast cancer, prostate cancer, liver cancer, brain cancer, lymphoma, leukemia, cervical cancer, skin cancer, pancreatic cancer, colorectal cancer, lung cancer and the like. In some embodiments, the cancer is a lung cancer. In some embodiments, the lung cancer is small-cell lung cancer (SCLC). In some embodiments, the SCLC is a KP1 small-cell lung cancer. In some embodiments, the SCLC is a KP2 small-cell lung cancer.

Other cancers that can be suitable treated with the compositions and methods of the present disclosure include, but are not limited to, acute myeloblastic leukemia (AML), acute lymphoblastic leukemia (ALL), chronic myelocytic leukemia (CML), adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, colon and rectum cancer, endometrial cancer, esophagus cancer, Ewing’s family of tumors (e.g. Ewing’s sarcoma), eye cancer, transitional cell carcinoma, vaginal cancer, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, gallbladder cancer, gastrointestinal carcinoid tumors, gastrointestinal stromal tumors, gestational trophoblastic disease, Non-Hodgkin’s lymphoma, Hodgkin’s lymphoma, childhood Non-Hodgkin’s lymphoma, Kaposi’s sarcoma, kidney cancer, laryngeal and hypopharyngeal cancer, liver cancer, lung cancer, lung carcinoid tumors, malignant mesothelioma, multiple myeloma, myelodysplastic syndrome, rhabdomyosarcoma, salivary gland cancer, sarcomas, melanoma skin cancer, non-melanoma skin cancers, stomach cancer, testicular cancer, thymus cancer, thyroid cancer, uterine cancer (e.g., uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom’s macroglobulinemia.

Particularly suitable cancers include, but are not limited to, breast cancer, ovarian cancer, lung cancer, pancreatic cancer, mesothelioma, leukemia, lymphoma, brain cancer, prostate cancer, multiple myeloma, melanoma, bladder cancer, bone sarcomas, soft tissue sarcomas, retinoblastoma, renal tumors, neuroblastoma, and carcinomas.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the subject inhibits metastasis of the cancer in the subject. For example, in some embodiments, the composition administered to the subject can reduce metastatic nodules in the subject. In some embodiments, the administered composition inhibits tumor growth in the subject.

In some embodiments, the proliferative disease is an autoimmune disease. In some embodiments, the autoimmune disease is selected from the group consisting of rheumatoid arthritis, insulin-dependent diabetes mellitus, hemolytic anemias, rheumatic fever, thyroiditis, Crohn’s disease, myasthenia gravis, glomerulonephritis, autoimmune hepatitis, multiple sclerosis, alopecia areata, psoriasis, vitiligo, dystrophic epidermolysis bullosa, systemic lupus erythematosus, moderate to severe plaque psoriasis, psoriatic arthritis, ulcerative colitis, graft vs. host disease, and diabetic foot ulcer.

In some embodiments, the administered composition inhibits proliferation of a target cancer cell, and/or inhibits tumor growth of the cancer in the subject. For example, the target cell may be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

In some embodiments, the disclosed therapeutic composition is formulated to be compatible with its intended route of administration. For example, the multivalent polypeptides multivalent antibodies, and vaccines of the disclosure may be given orally or by inhalation, but it is more likely that they will be administered through a parenteral route. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as mono- and/or dibasic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject multivalent polypeptides and multivalent antibodies of the disclosure can be determined by standard pharmaceutical procedures in cell cultures or experimental animals, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are generally suitable. While compounds that exhibit toxic side effects may be used, care should be taken to design a delivery system that targets such compounds to the site of affected tissue in order to minimize potential damage to uninfected cells and, thereby, reduce side effects.

For example, the data obtained from the cell culture assays and animal studies can be used in formulating a range of dosage for use in humans. The dosage of such compounds lies generally within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

The therapeutic compositions described herein, e.g., multivalent polypeptides, multivalent antibodies, nucleic acids, recombinant cells, cell cultures, vaccines and pharmaceutical compositions, can be administered one from one or more times per day to one or more times per week; including once every other day. The skilled artisan will appreciate that certain factors may influence the dosage and timing required to effectively treat a subject, including but not limited to the severity of the disease, previous treatments, the general health and/or age of the subject, and other diseases present. Moreover, treatment of a subject with a therapeutically effective amount of the subject multivalent polypeptides and multivalent antibodies of the disclosure can include a single treatment or, can include a series of treatments. In some embodiments, the compositions are administered every 8 hours for five days, followed by a rest period of 2 to 14 days, e.g., 9 days, followed by an additional five days of administration every 8 hours. With regard to multivalent polypeptides or multivalent antibodies, the therapeutically effective amount of a multivalent polypeptide or multivalent antibody of the disclosure (e.g., an effective dosage) depends on the multivalent polypeptide or multivalent antibody selected. For instance, single dose amounts in the range of approximately 0.001 to 0.1 mg/kg of patient body weight can be administered; in some embodiments, about 0.005, 0.01, 0.05 mg/kg may be administered.

As discussed above, some embodiments of the disclosure relate to methods for modulating cell signaling mediated by CD47 and/or SIRPα. The method is performed by administering to the subject a composition including one or more of: (i) a multivalent polypeptide of the disclosure, (ii) a multivalent antibody of the disclosure, (iii) a recombinant nucleic acid molecule of the disclosure, and (iv) and/or a recombinant cell of the disclosure. In another aspect, some embodiments of the disclosure relate to methods for the treatment of a health condition in a subject in need thereof. The method is performed by administering to the subject a composition including one or more of: (i) a multivalent polypeptide of the disclosure, (ii) a multivalent antibody of the disclosure, (iii) a recombinant nucleic acid molecule of the disclosure, and (iv) and/or a recombinant cell of the disclosure. In some embodiments, the methods are performed by administering to the subject an effective amount of therapeutic composition as disclosed herein.

As discussed supra, a therapeutically effective amount includes an amount of a therapeutic composition that is sufficient to promote a particular effect when administered to a subject, such as one who has, is suspected of having, or is at risk for a health condition, e.g., a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The efficacy of a treatment including a disclosed therapeutic composition for the treatment of disease can be determined by the skilled clinician. However, a treatment is considered effective treatment if at least any one or all of the signs or symptoms of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

In some embodiments of the disclosed methods, the administered composition, e.g., multivalent polypeptide or multivalent antibody of the disclosure or nucleic acid encoding the same, recruits a RPTP activity into spatial proximity of a SIRPα molecule present on the surface of a cell, eliciting phosphatase activity that reduces the phosphorylation level of the SIRPα molecule. In some embodiments, the administered multivalent polypeptide recruits the RPTP into spatial proximity of a SIRPα molecule present on the surface of the same cell as the RPTP, e.g., the distance between the intracellular domain of the RPTP and the intracellular domain of the SIRPα molecule, in cis (e.g., the RPTP and the SIRPα molecule, are present in the same cell), is less than about 500 angstroms, such as e.g., a distance of about 5 angstroms to about 500 angstroms. In some embodiments, the spatial proximity amounts to less than about 5 angstroms, less than about 20 angstroms, less than about 50 angstroms, less than about 75 angstroms, less than about 100 angstroms, less than about 150 angstroms, less than about 250 angstroms, less than about 300 angstroms, less than about 350 angstroms, less than about 400 angstroms, less than about 450 angstroms, or less than about 500 angstroms. In some embodiments, the spatial proximity amounts to less than about 100 angstroms. In some embodiments, the spatial proximity amounts to less than about 50 angstroms. In some embodiments, the spatial proximity amounts to less than about 20 angstroms. In some embodiments, the spatial proximity amounts to less than about 10 angstroms. In some embodiments, the spatial proximity ranges from about 10 to 100 angstroms, from about 50 to 150 angstroms, from about 100 to 200 angstroms, from about 150 to 250 angstroms, from about 200 to 300 angstroms, from about 250 to 350 angstroms, from about 300 to 400 angstroms, from about 350 to 450 angstroms, or about 400 to 500 angstroms. In some embodiments, the administered multivalent polypeptide or the multivalent antibody recruits the RPTP into spatial proximity such that the RPTP is about 10 to 100 angstroms from the SIRPα molecule. In some embodiments, the spatial proximity amounts to less than about 100 angstroms. In some embodiments, the distance between the intracellular domain RPTP and the intracellular domain of the SIRPα molecule, in cis, is less than about 250 angstroms, alternatively less than about 200 angstroms, alternatively less than about 150 angstroms, alternatively less than about 120 angstroms, alternatively less than about 100 angstroms, alternatively less than about 80 angstroms, alternatively less than about 70 angstroms, or alternatively less than about 50 angstroms.

The term “modulating”, in relation to the cell signaling pathway mediated by CD47 and/or SIRPα refers to a change in the cell signaling pathway. Modulation includes both increase (e.g., promote, enhance, induce, stimulate) and decrease (e.g., reduce, inhibit, suppress), or otherwise affecting the cell signaling pathway. In some embodiments of the disclosed methods, the administered composition, e.g., multivalent polypeptide or multivalent antibody of the disclosure or nucleic acid encoding the same, recruits the RPTP activity to a spatial proximity of SIRPα, potentiates dephosphorylation of SIRPα, reduces SIRPα-mediated signaling, promotes macrophage phagocytosis, and/or promotes dendritic cell maturation.

In some embodiments, when the RPTP molecule and the SIRPα molecule are brought into a spatial proximity of one to another, the phosphorylation level of the SIRPα molecule can be reduced by at least, or at least about, 10%, 15%, 20%, 25%, 30%, 35%, 40%, 45%, 50%, 55%, 60%, 65%, 70%, 75%, 80%, 85%, 90%, 95%, or 100%, or a range of any two of the proceeding values, for example from about 20% to about 60% (inclusive of values in between these percentages), as compared to the phosphorylation level of the SIRPα molecule in an untreated subject under similar conditions.

In some embodiments, the administration of a composition of the disclosure (e.g., multivalent polypeptide or multivalent antibody or nucleic acid encoding the same) confers a reduced activity of SIRPα-mediated signaling in the subject. The reduction in activity of SIRPα-mediated signaling can be reduced by at least, or at least about, 10%, 15%, 20%, 25%, 30%, 35%, 40%, 45%, 50%, 55%, 60%, 65%, 70%, 75%, 80%, 85%, 90%, 95%, or 100%, or a range of any two of the proceeding values, for example from about 20% to about 60% (inclusive of values in between these percentages), as compared to the activity of SIRPα-mediated signaling in an untreated subject under similar conditions.

In some embodiments of the disclosed methods, the administration of the multivalent polypeptide or the multivalent antibody confers an enhancement in macrophage activity in the subject. The macrophage activity can be enhanced by at least, or at least about, 10%, 15%, 20%, 25%, 30%, 35%, 40%, 45%, 50%, 55%, 60%, 65%, 70%, 75%, 80%, 85%, 90%, 95%, or 100%, or a range of any two of the proceeding values, for example from about 20% to about 60% (inclusive of values in between these percentages), as compared to the macrophage activity in an untreated subject under similar conditions.

In some embodiments of the disclosed methods, the subject is a mammal. In some embodiments, the mammal is human. In some embodiments, the subject has or is suspected of having a health condition associated with inhibition of cell signaling mediated by CD47 and/or SIRPα. The health condition suitable for being treated by the compositions and methods of the disclosure include, but are not limited to, cancers, autoimmune diseases, inflammatory diseases, and infectious diseases. In some embodiments, the disease is a cancer or a chronic infection.

According to yet a further aspect of the disclosure, there is provided a method of preventing or treating a health condition, e.g., cancer in a subject, the method including the steps of: (i) culturing immature DCs with a cancer-associated antigen so as to produce tumor antigen-presenting dendritic cells; (ii) maturing the dendritic cells according to a process substantially as described above to produce mature DCs; and (iii) administering the subject with the mature antigen-presenting dendritic cells. In some embodiments, the dendritic cell is a bone marrow-derived dendritic cell (BMDC).

According to yet a further aspect of the disclosure, there is provided a method of preventing or treating an infection by a parasite, virus, micro-fungus, bacterium in a subject, the method including the steps of: (i) culturing immature DCs with a parasite-, virus-, microfungus-, bacterium-associated antigen so as to produce antigen-presenting dendritic cells; (ii) maturing the dendritic cells according to a process substantially as described above to produce mature DCs; and (iii) administering the subject with the mature antigen-presenting dendritic cells. In some embodiments, the dendritic cell is a bone marrow-derived dendritic cell (BMDC).

### Additional Therapies

As discussed supra, any one of the compositions disclosed herein, e.g., multivalent polypeptides, multivalent antibodies, nucleic acids, recombinant cells, cell cultures, and/or pharmaceutical compositions described herein can be administered to a subject in need thereof as a single therapy (e.g., monotherapy). In addition or alternatively, in some embodiments of the disclosure, the multivalent polypeptides, multivalent antibodies, nucleic acids, recombinant cells, cell cultures, and/or pharmaceutical compositions described herein can be administered to the subject in combination with one or more additional therapies, e.g., at least one, two, three, four, or five additional therapies. Suitable therapies to be administered in combination with the compositions of the disclosure include, but are not limited to chemotherapy, radiotherapy, immunotherapy, hormonal therapy, toxin therapy, targeted therapy, and surgery. Other suitable therapies include therapeutic agents such as chemotherapeutics, anti-cancer agents, and anti-cancer therapies.

Administration “in combination with” one or more additional therapies includes simultaneous (concurrent) and consecutive administration in any order. In some embodiments, the one or more additional therapies is selected from the group consisting of chemotherapy, radiotherapy, immunotherapy, hormonal therapy, toxin therapy, and surgery. The term chemotherapy as used herein encompasses anti-cancer agents. Various classes of anti-cancer agents can be suitably used for the methods disclosed herein. Non-limiting examples of anti-cancer agents include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

Topoisomerase inhibitors are also another class of anti-cancer agents that can be used herein. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins such as irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

Antineoplastics include the immunosuppressant dactinomycin, doxorubicin, epirubicin, bleomycin, mechlorethamine, cyclophosphamide, chlorambucil, ifosfamide. The antineoplastic compounds generally work by chemically modifying a cell’s DNA.

Alkylating agents can alkylate many nucleophilic functional groups under conditions present in cells. Cisplatin and carboplatin, and oxaliplatin are alkylating agents. They impair cell function by forming covalent bonds with the amino, carboxyl, sulfhydryl, and phosphate groups in biologically important molecules.

Vinca alkaloids bind to specific sites on tubulin, inhibiting the assembly of tubulin into microtubules (M phase of the cell cycle). The vinca alkaloids include: vincristine, vinblastine, vinorelbine, and vindesine.

Anti-metabolites resemble purines (azathioprine, mercaptopurine) or pyrimidine and prevent these substances from becoming incorporated in to DNA during the “S” phase of the cell cycle, stopping normal development and division. Anti-metabolites also affect RNA synthesis.

Plant alkaloids and terpenoids are obtained from plants and block cell division by preventing microtubule function. Since microtubules are vital for cell division, without them, cell division cannot occur. The main examples are vinca alkaloids and taxanes.

Podophyllotoxin is a plant-derived compound which has been reported to help with digestion as well as used to produce two other cytostatic drugs, etoposide and teniposide. They prevent the cell from entering the G1 phase (the start of DNA replication) and the replication of DNA (the S phase).

Taxanes as a group includes paclitaxel and docetaxel. Paclitaxel is a natural product, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

In some embodiments, the anti-cancer agents can be selected from remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, gefitinib (Iressa®), taxol, taxotere, fluorouracil, leucovorin, irinotecan, xeloda, CPT-11, interferon alpha, PEGylated interferon alpha (e.g., PEG INTRON-A), capecitabine, cisplatin, thiotepa, fludarabine, carboplatin, liposomal daunorubicin, cytarabine, doxetaxol, paclitaxel, vinblastine, interleukin 2 (IL-2), Granulocyte-macrophage colony-stimulating factor (GM-CSF), dacarbazine, vinorelbine, zoledronic acid, palmitronate, biaxin, busulphan, prednisone, bortezomib (Velcade®), bisphosphonate, arsenic trioxide, vincristine, doxorubicin (Doxil®), paclitaxel, ganciclovir, adriamycin, estrainustine sodium phosphate (Emcyt®), sulindac, etoposide, and combinations of any thereof.

In other embodiments, the anti-cancer agent can be selected from bortezomib, cyclophosphamide, dexamethasone, doxorubicin, interferon-alpha, lenalidomide, melphalan, pegylated interferon-alpha, prednisone, thalidomide, or vincristine.

In some embodiments, the methods of treatment as described herein further include an immunotherapy. In some embodiments, the immunotherapy includes administration of one or more checkpoint inhibitors. Accordingly, some embodiments of the methods of treatment described herein include further administration of a compound that inhibits one or more immune checkpoint molecules. Non-limiting examples of immune checkpoint molecules include CTLA4, PD-1, PD-L1, A2AR, B7-H3, B7-H4, TIM3, and combinations of any thereof. In some embodiments, the compound that inhibits the one or more immune checkpoint molecules includes an antagonistic antibody. Examples of antagonistic antibodies suitable for the compositions and methods disclosed herein include, but are not limited to, ipilimumab, nivolumab, pembrolizumab, durvalumab, atezolizumab, tremelimumab, and avelumab.

In some aspects, the one or more anti-cancer therapy is radiation therapy. In some embodiments, the radiation therapy can include the administration of radiation to kill cancerous cells. Radiation interacts with molecules in the cell such as DNA to induce cell death. Radiation can also damage the cellular and nuclear membranes and other organelles. Depending on the radiation type, the mechanism of DNA damage may vary as does the relative biologic effectiveness. For example, heavy particles (i.e. protons, neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray. Radiation also contemplated herein includes, for example, the directed delivery of radioisotopes to cancer cells. Other forms of DNA damaging factors are also contemplated herein such as microwaves and UV irradiation.

Radiation may be given in a single dose or in a series of small doses in a dose-fractionated schedule. The amount of radiation contemplated herein ranges from about 1 to about 100 Gy, including, for example, about 5 to about 80, about 10 to about 50 Gy, or about 10 Gy. The total dose may be applied in a fractioned regime. For example, the regime may include fractionated individual doses of 2 Gy. Dosage ranges for radioisotopes vary widely, and depends on the half-life of the isotope and the strength and type of radiation emitted. When the radiation includes use of radioactive isotopes, the isotope may be conjugated to a targeting agent, such as a therapeutic antibody, which carries the radionucleotide to the target tissue (e.g., tumor tissue).

Surgery described herein includes resection in which all or part of a cancerous tissue is physically removed, exercised, and/or destroyed. Tumor resection refers to physical removal of at least part of a tumor. In addition to tumor resection, treatment by surgery includes laser surgery, cryosurgery, electrosurgery, and microscopically controlled surgery (Mohs surgery). Removal of pre-cancers or normal tissues is also contemplated herein.

Accordingly, in some embodiments, the methods of the disclosure include administration of a composition disclosed herein to a subject individually as a single therapy (e.g., monotherapy). In some embodiments, a composition of the disclosure is administered to a subject as a first therapy in combination with a second therapy. In some embodiments, the second therapy is selected from the group consisting of chemotherapy, radiotherapy, immunotherapy, hormonal therapy, toxin therapy, and surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### KITS

Also provided herein are kits for the practice of a method described herein. A kit can include instructions for use thereof and one or more of the multivalent polypeptides, multivalent antibodies, nucleic acids, recombinant cells, and pharmaceutical compositions disclosed herein as described and provided herein. For examples, provided herein, in some embodiments, are kits that include one or more multivalent polypeptides and/or multivalent antibodies of the disclosure, and instructions for use thereof. In some embodiments, provided herein are kits that include one or more nucleic acids, recombinant cells, and/or pharmaceutical compositions of the disclosure; and instructions for use thereof. In some embodiments, the kits of disclosure further include written instructions for preparing the multivalent polypeptides, multivalent antibodies, nucleic acids, recombinant cells, and pharmaceutical compositions of the disclosure and using the same.

In some embodiments, the kits of the disclosure further include one or more syringes (including pre-filled syringes) and/or catheters (including pre-filled syringes) used to administer one any of the provided immune cells, nucleic acids, and pharmaceutical compositions to a subject in need thereof. In some embodiments, a kit can have one or more additional therapeutic agents that can be administered simultaneously or sequentially with the other kit components for a desired purpose, e.g., for modulating cell signaling mediated by CD47 and/or SIRPα, or preventing or treating a health condition in a subject in need thereof.

For example, any of the above-described kits can further include one or more additional reagents, where such additional reagents can be selected from: dilution buffers; reconstitution solutions, wash buffers, control reagents, control expression vectors, negative control T-cell populations, positive control T-cell populations, reagents for ex vivo production of the T-cell populations.

In some embodiments, the components of a kit can be in separate containers. In some other embodiments, the components of a kit can be combined in a single container. For example, in some embodiments of the disclosure, the kit includes one or more of the multivalent polypeptides, multivalent antibodies, nucleic acids, recombinant cells, and pharmaceutical compositions disclosed herein in one container (e.g., in a sterile glass or plastic vial) and a further therapeutic agent in another container (e.g., in a sterile glass or plastic vial).

In some embodiments, a kit can further include instructions for using the components of the kit to practice a method described herein. For example, the kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and intellectual property information.

In some embodiments, a kit can further include instructions for using the components of the kit to practice the methods. The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

Additional embodiments are disclosed in further detail in the following examples, which are provided by way of illustration and are not in any way intended to limit the scope of this disclosure or the claims.

### Example 1. General Experimental Procedures

### Cell Lines

Cell lines were kept in a humidified incubator at 37° C. with 5% CO2 unless otherwise denoted. HEK293T (LentiX) cells (female derived kidney cell line) were grown in DMEM complete media (Thermo Fisher) supplemented with 10% FBS, 2 mM L-glutamine, and 50 U/ml of penicillin and streptomycin. MC38 were purchased from Kerafast, and cultured in DMEM complete media containing 10% FBS, 2 mM L-glutamine, 0.1 mM NEAA, 1 mM NaPyr, 10 mM HEPES, 50 U/ml P/S, 50 µg/ml gentamycin sulfate.

### Animals

All animals were housed at Stanford University according to protocol and guidelines approved by the Administrative Panel on Lab Animal Care (APLAC). C57BL/6J mice were purchased from Jackson Labs (Cat 000664). B6.Cg-Foxp3tm2Tch/J (B6.FoxP3GFP, Cat. #006772), and B6.Cg-Thy1a/Cy Tg(TcraTcrb)8Rest/J (pmel-1 TCR tg mice, Cat. #005023) were purchased from Jackson Labs and bred in house.

### Protein Expression

Insect Tni cells (Expression Systems, cat. #94-002S) were grown in Insect X-press media (Lonza) or ESF 921 media (Expression Systems) with a final concentration of 10 mg/L of gentamicin sulfate (Thermo Fisher) at 27° C. and atmospheric CO₂. SF9 cells (Thermo Fisher Scientific) were grown in SF900-III or -II serum-free media (Thermo Fisher) with 10% FBS and final concentration 10 mg/L of gentamicin sulfate and 2 mM Glutamax at 27° C. and atmospheric CO₂. P1 or P2 virus was used to infect volumes of 1-3 L of Hi5 cells at ~2 × 10⁶ cells/ml. New P1 or P2 preps were made from fresh P0 batches routinely. Supernatant was harvested 2-3 days post-infection and spun down at 8000 rpm for 15 minutes. The supernatant containing expressed protein was treated to 100 mM Tris pH 8.0, 2 mM NiCl₂, and 10 mM CaCl₂ to precipitate contaminants. The supernatant and precipitate mixture was spun down at 8000 rpm for 20 min at 4° C. to remove precipitate. The supernatant was incubated with Ni-NTA resin (QIAGEN) for > 3 hours at room temperature. Ni-NTA beads were collected and washed in a Buchner funnel with 20 mM imidazole in 1×HBS pH 7.2 and eluted with 200 mM imidazole in 1 × HBS pH 7.2. Protein was concentrated in a 10 kDa filter (Millipore, UFC903024) to ~1 mL or until 10 mg/ml. When appropriate, proteins were biotinylated with BirA ligase, 100 µM biotin, 40 mM Bicine pH 8.3, 10 mM ATP, and 10 mM Magnesium Acetate at 4° C. overnight. All proteins were further purified by size-exclusion chromatography using Superdex Increase S200 or S75, as appropriate (GE Healthcare). All proteins for in vivo studies were cleared of endotoxin. Final endotoxin levels were determined using a chromogenic endotoxin quantitation kit (Thermo Fisher) and never greater than 1 Endotoxin Unit/mg of purified protein. RIPR proteins were kept at 4° C. for up to two weeks to prevent freeze/thawing cycles.

### PBMC Stimulation

PBMCs were obtained from the Stanford Blood Bank. Cells in de-identified leukoreduction chambers from healthy platelet donors were processed as soon as possible and no later than 18 hours after plateletpheresis. PBMCs were stimulated with plate bound OKT3 and CD28 (as described above) or with 20 µM of CEFX Ultra SuperStim Pool, from the PepMix® Peptide Pool series (JPT Peptide Technologies GmbH, Germany). For peptide stimulations, cells were treated with a second dose of 20 µM of CEFX Ultra SuperStim Pool 24 hours after the first incubation. At this time point, cells were incubated with antibodies or RIPR or CD45 diabodies or appropriate controls. Cells and supernatant were collected 24, 48 and 72 hours post incubation with antibodies, RIPR or appropriate molecules.

### Example 2. RIPR-SIRPα Design and Expression

Two RIPR-SIRPα molecules were developed. First generation RIPR-SIRPα was composed from an anti-CD45 scFv (clone #4, as described previously in WO2005/026210) fused to “Velcro”, a high affinity CD47 variant molecule (see, e.g., FIGS. 2A-2C). High affinity variant Velcro-CD47 has been reported to bind the two most prominent human SIRPα alleles with greatly increased affinity relative to wild-type CD47 and potently antagonized CD47 binding to SIRPα on human macrophages (Ho C.C. et al., 2015). In these constructs, a Gly-Ser linker sequence or a GGSLEVLFQGPGSGS (SEQ ID NO: 10) encoding a 3C cleavage site was inserted in between the anti-CD45 scFv sequence and the Velcro.

The second generation RIPR-SIRPα was composed from the same anti-CD45 scFv fused to an anti-SIRPα scFv, which is clone AB21 described in Sim J. et al. MAbs, 2019, Vol. No. 6, pp. 1036-1052. As shown in FIG. 2B, a 3C cleavage site was inserted in the linker region connecting the anti-CD45 and anti-SIRPα arms. RIPR-SIRPα proteins were produced in Tni cells as described previously in WO2019222547A1. Velcro was produced as described previously (Ho C.C. et al., supra, 2015). The sequences corresponding to the AB21 Fab were ordered as gblocks, cloned in the appropriate plasmids for protein expression in Expi293 cells.

All proteins were purified using Ni-NTA and the fractions corresponding to a monodisperse peak after SEC were pooled and concentrated. Protein integrity was further confirmed by reduced and non-reducing SDS-PAGE electrophoresis followed by Coomassie blue staining. Protein was kept at 4° C. for immediate use or stored frozen at -80° C.

### Example 3. SIRPα Phosphorylation and Phagocytosis Assay

To reconstitute the SIRPα phosphorylation, approximately 4×10⁶ HEK293 cells were transiently transfected with plasmids encoding full length human Lck, CD45, CD45Dead or SIRPα at an optimized ratio. Cells were treated with RIPR-SIRPα 24 hours after transfection for 30 min at 37° C. As a control, RIPR-SIRPα was treated with 3C enzyme (100 µg/mL) for 14 hours at 4° C. Cleavage was analyzed by Coomassie blue staining after SDS-PAGE electrophoresis. Cleaved RIPR-SIRPα was added to the cells for 30 min at 37° C. After treatment, cells were harvested and cell lysates were incubated with anti-HA magnetic beads (Pierce, Thermo Fisher Scientific) for immunoprecipitation. Cells lysates were analyzed by Western blot for HA, SIRPα and phosphotyrosine. To quantify endogenous SIRPα phosphorylation levels, approximately 1×10⁷ THP-1 macrophages were incubated with 100 nM of AB21 Fab or RIPR-SIRPα for 30 minutes at 37° C. after which cells were harvested and cell lysates were incubated with 5 µg of anti-SIRPα antibody for 1 hour at 4° C. and incubated overnight with 30 µL of Protein A/G magnetic beads (Pierce, Thermo Fisher Scientific). All cells were lysed in Pierce IP Lysis Buffer (Pierce Thermo Fisher Scientific, #87787) supplemented with 2X Phosphatase Inhibitor Cocktail (Abcam and Promega), Protease Inhibitor Cocktail Tablet (Roche), Orthovanadate (NEB), 2 mM EDTA and 1% (w/v) of n-Dodecyl β-D-maltoside (Anatrace). After immunoprecipitation, SIRPα phosphorylation was analyzed by Western blot. For the phagocytosis assay, approximately 5×10⁴ human PBMC macrophages were pretreated with or without human RIPR-SIRPα, Velcro or anti-SIRPα Fab, clone AB21 for 30 min at 37° C., and ~1×10⁵ human tumor Raji B cells were pretreated with varying Rituximab (from 0 to 5 µg/mL) for 30 min at 37° C. After incubation, the macrophage cells were co-cultured with 1×10⁴ CFSE labelled Raji B cells for 2 hours at 37° C. Cells were harvested and stained with the macrophage marker CD11b for 20 min at 4° C. and analyzed by a CytoFLEX Flow Cytometer.

### Example 4. “Vecro” SIRPα-RIPR Can Robustly Reduce SIRPα Tyrosine Phosphorylation

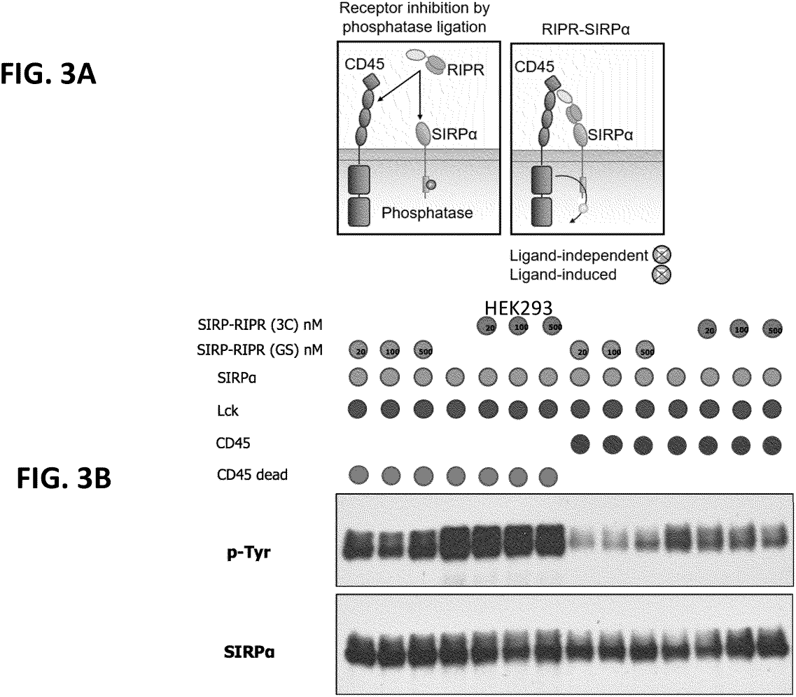

This Example describes the results of experiments performed to demonstrate that a “Vecro” SIRPα-RIPR multivalent polypeptide designed in accordance with some embodiments of the disclosure can robustly reduce SIRPα tyrosine phosphorylation. As shown in FIG. 3B, in these experiments, HEK293 cells were transiently transfected with target receptor human HA-SIRPα, Lck and human CD45, 24 hours after transfection, cells were left untreated (lane 4) or incubated for 20 min at 37° C. with SIRPα-RIPR(GS) (lane 1, 2 and 3) or SIRPα-RIPR(3C) (lane 5, 6 and 7) to induce in cis recruitment of the CD45 intracellular domain to the intracellular domains of SIRPα (e.g., the RPTP and the SIRPα molecule are present in the same cell). A CD45 phosphatase-deficient group was included for control purposes (CD45 dead; C853S). After lysis, chimeric receptors were immunoprecipitated with anti-HA antibody directly conjugated to magnetic beads. Samples were probed for phosphotyrosine (pTyr) and SIRPα by western blot. Data are representative of three independent biological repeats.

### Example 5. Human SIRPα-RIPR Ligands Enhance Rituximab Mediated ADCP

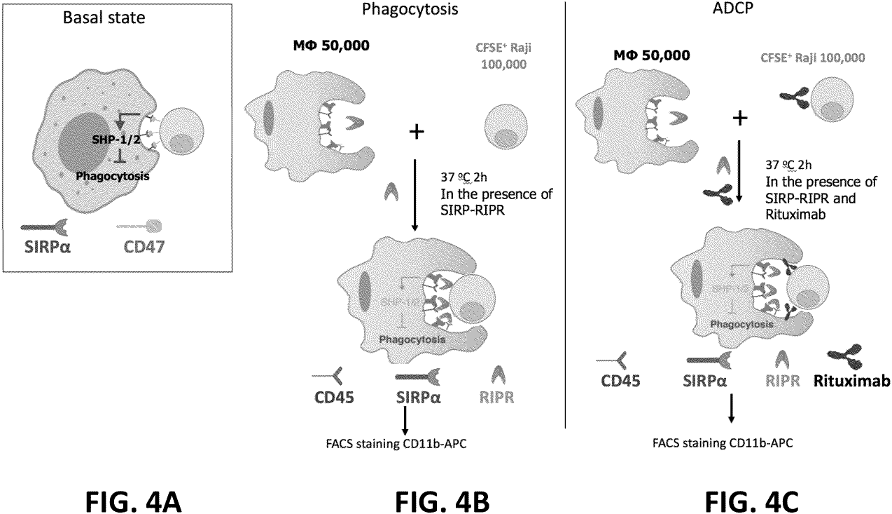

This Example describes experiments performed to demonstrate that SIRPα-RIPR multivalent polypeptide designed in accordance with some embodiments of the disclosure can antibody-dependent cell-mediated cytotoxicity (ADCP). Non-limiting phagocytosis assays were designed to test SIRPα-RIPR function on phagocytosis and antibody-dependent cell-mediated cytotoxicity (ADCP) (FIGS. 4A-4C). FIG. 4A shows schematic depiction of CD47-SIRPα “don’t eat me” signal axis in macrophage. In the basal state, recruitment of CD47 to tumor cells with SIRPα on macrophages results in SHP1 and 2 recruitment and activation and inhibits phagocytosis. FIG. 4B shows schematics of phagocytosis assay for testing the effect of SIRPα-RIPR. SIRPα-RIPR silencing the SIRP signaling by recruiting CD45, thereby disinhibiting phagocytosis. FIG. 4C shows schematics of antibody dependent cellular phagocytosis assay for testing the effect of SIRPα-RIPR. SIRPα-RIPR silencing the SIRPα signaling by recruiting CD45.

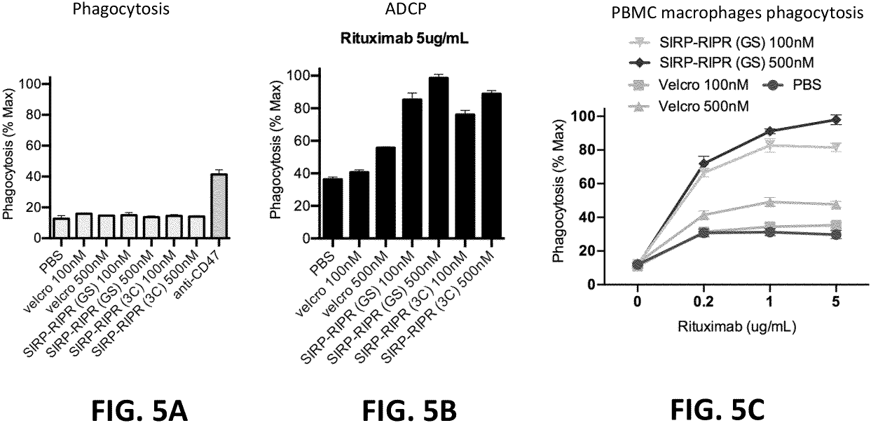

The results of experiments performed to demonstrate that human SIRPα-RIPR ligands enhance Rituximab-mediated ADCP are shown in FIGS. 5A-5C. Human SIRPα-RIPR ligands enhance Rituximab mediated ADCP. Phagocytosis assay for testing SIRPα-RIPR. 5×10⁴ human macrophages were pretreated with ‘Velcro’, human SIRPα-RIPR(GS) or SIRPα-RIPR(3C) for 30 min at 37° C., the macrophages were co-cultured with 1×10⁵ Raji cells (CFSE labelled) for 2 hours at 37° C. Anti CD47 was included for a positive control (FIG. 5A). ADCP assay for testing SIRPα-RIPR is shown in FIG. 5B. 5×10⁴ human macrophages were pretreated with ‘Velcro’, human SIRPα-RIPR(GS) or SIRPα-RIPR(3C) for 30 min at 37° C., 1×10⁵ Raji cells were pretreated with or without 5 µg/mL anti-CD20 antibody (Rituximab) for 30 min at 37° C., the macrophages were co-cultured with Raji cells for 2 hours at 37° C. The cells were harvested and stained with CD11b for 20 min at 4° C. Phagocytosis was quantified by flow cytometry. Data are mean ± SD from n = 2 biological replicates from 1 representative of 2 independent experiments. In FIG. 5C, human macrophages were pretreated with or without human SIRPα-RIPR for 30 min at 37° C., Raji cells were pretreated with varying Rituximab (from 0 to 5 µg/mL) for 30 min at 37° C., the macrophages were co-cultured with Raji cells for 2 hours at 37° C. Phagocytosis was quantified by flow cytometry. Data are mean ± SD from n = 2 biological replicates from 1 representative of 2 independent experiments.

### Example 6. SIRPα-RIPR Bispecific Antibody Potentiates Dephosphorylation of Human SIRPα

This Example describes experiments performed to demonstrate that an exemplary SIRPα-RIPR bispecific antibody in accordance with some embodiments of the disclosure can potentiates dephosphorylation of human SIRPα.

A non-limiting example of a bispecific antibody SIRPα-RIPR design in accordance with some embodiments of the disclosure is shown in FIG. 6B. FIG. 6B also shows schematic representation of AB21 and AB21 based human SIRPα-RIPR molecules and the amino acid sequences of AB21, SIRPα-RIPR with a GS linker (e.g., GGGGTGGS; SEQ ID NO: 9), SIRPα-RIPR with a 3C linker (LEVLFQGP; SEQ ID NO: 11). Affinity binding of antibody AB21 to human SIRPα and mouse SIRPα from different mouse strains (PCT Publication No. WO2018057669A1) is shown in FIG. 6A.

The results of experiments performed to demonstrate that AB21 SIRPα-RIPR potentiates dephosphorylation of human SIRPα. AB21 SIRPα-RIPR potentiates dephosphorylation of human SIRPα are shown in FIGS. 7A-7C. Schematic depiction of SIRPα-RIPR mechanism is shown in FIG. 7A. Schematic depiction of bispecific diabody to CD45 and SIRPα is shown in FIG. 7B. HEK293 cells were transiently transfected with human HA-SIRPα, Lck and human CD45, 24 hours after transfection, cells were left untreated (lane 1) or incubated for 20 min at 37° C. with Ab21 (lane 2 and 3) or SIRPα-RIPR(GS) (lane 4, 5) to induce in cis recruitment of the CD45 phosphatase to the intracellular domains of SIRPα. A CD45 dead group was included for control purposes. After lysis, chimeric receptors were immunoprecipitated with anti-HA antibody directly conjugated to magnetic beads. Samples were probed for pTyr and SIRPα by western blot (FIG. 7C). Data are representative of three independent biological repeats.

### Example 7. SIRPα-RIPR Reduces SIRPα Tonic Signaling and Enhances ADCP of Human Macrophages

This Example describes the results of experiments performed to demonstrate that SIRPα-RIPR reduces SIRPα signaling and enhances ADCP of human macrophages. SIRPα phosphorylation after immunoprecipitation from resting THP1 macrophages were detected (FIG. 8A). THP1 macrophages were incubated with 500 nM AB21, or SIRPα-RIPR for 30 min at 37° C. prior to SIRPα IP. In vitro phagocytosis assay (ADCP) was performed using macrophages isolated from human PBMCs and incubated with Raji cells pretreated with Rituximab at the indicated concentrations (FIG. 8B) or at 1 µg/mL (FIG. 8C). Macrophage cells were incubated with target cells for 2 hours at 37° C. Phagocytosis was quantified by flow cytometry. Data are mean ± SD from n = 2 biological replicates from 1 representative of 2 independent experiments.

### Example 8. SIRPα-RIPR Reduces SIRPα Tonic Signaling and Enhances ADCP of Murine Macrophages

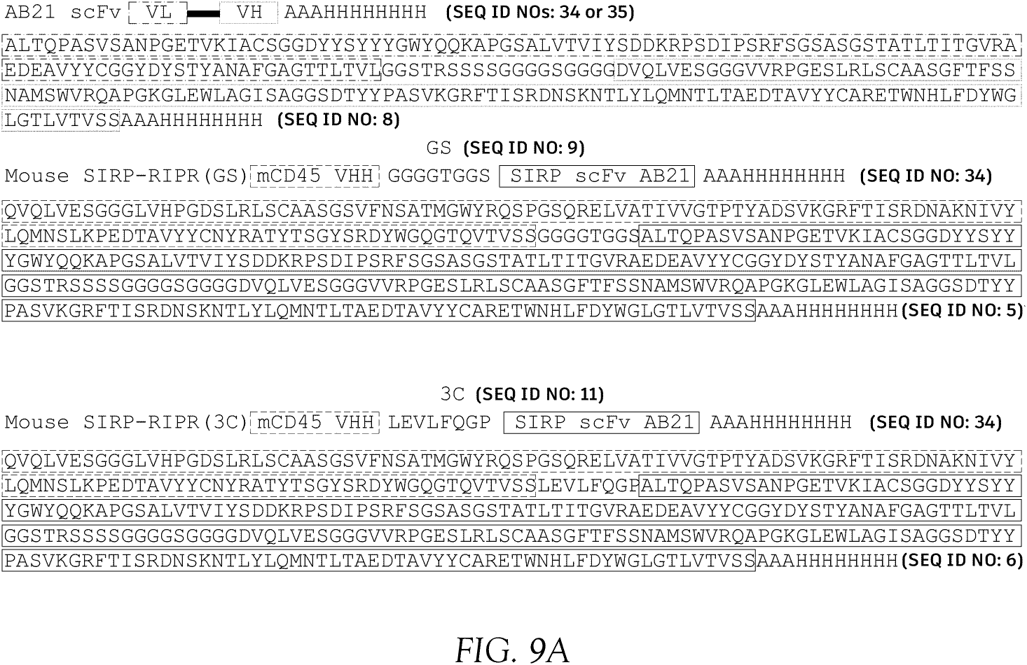

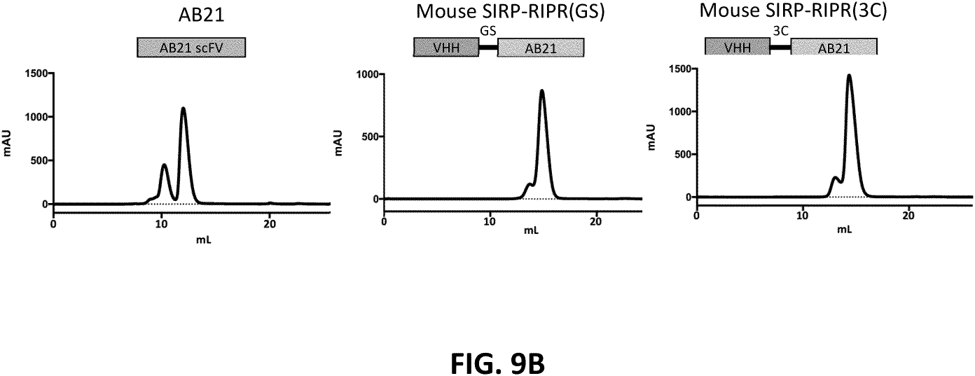

This Example describes the results of experiments performed to demonstrate that SIRPα-RIPR reduces SIRPα tonic signaling and enhances ADCP of murine macrophages. Another non-limiting example of a bispecific antibody SIRPα-RIPR design in accordance with some embodiments of the disclosure are illustrated in FIGS. 9A-9B. Schematic representation of AB21 and AB21 based mouse SIRPα-RIPR molecules and the amino acid sequence of AB21, SIRPα-RIPR with a GS linker, SIRPα-RIPR with a 3C linker (LEVLFQGP; SEQ ID NO: 11) are shown in FIG. 9A. AB21 and SIRPα-RIPRs were expressed in Hi5 cells. The proteins were analyzed by size-exclusion chromatography (FIG. 9B).

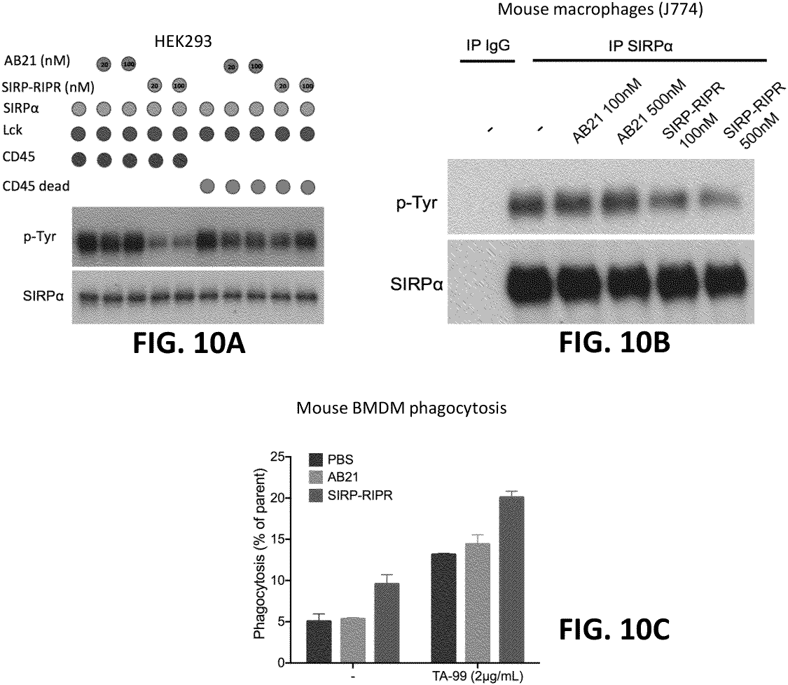

The results of experiments performed to demonstrate that the bispecific antibody SIRPα-RIPR described in FIGS. 9A-9B above can reduce SIRPα tonic signaling and enhances ADCP of mouse macrophages are summarized in FIGS. 10A-10C. As shown in FIGS. 10A-10C, SIRPα-RIPR reduced SIRPα signaling and enhanced ADCP of mouse macrophages. HEK293 cells were transiently transfected with mouse HA-SIRPα, Lck and mouse CD45, 24 hours after transfection, cells were left untreated (lane 1) or incubated for 30 min at 37° C. with Ab21 (lane 2 and 3) or SIRPα-RIPR(GS) (lane 4, 5) to induce in cis recruitment of the CD45 intracellular domain to the intracellular domains of SIRPα. A CD45 dead group was included for control purposes. After lysis, chimeric receptors were immunoprecipitated with anti-HA antibody directly conjugated to magnetic beads. Samples were probed for pTyr and SIRPα by western blot. Data are representative of three independent biological repeats (FIG. 10A). Detection of SIRPα phosphorylation after immunoprecipitation from resting J774 macrophages was detected. J774 macrophages were incubated with AB21, or mouse SIRPα-RIPR for 30 min at 37° C. prior to SIRPα IP (FIG. 10B). Mouse bone marrow derived macrophage (BMDM) cells were incubated with B16F10 (CFSE labeled) pretreated with or without 2 µg/mL anti TRP-1 mAb (TA99) for 2 hours at 37° C. Mouse CD47 nanobody A4 was included for control purposes. Phagocytosis was quantified by flow cytometry. Data are mean ± SD from n = 2 biological replicates from 1 representative of 2 independent experiments (FIG. 10C).

### Example 9. SIRPα-RIPR Enhances Maturation of Bone Marrow Derived Dendritic Cells (BMDCs)

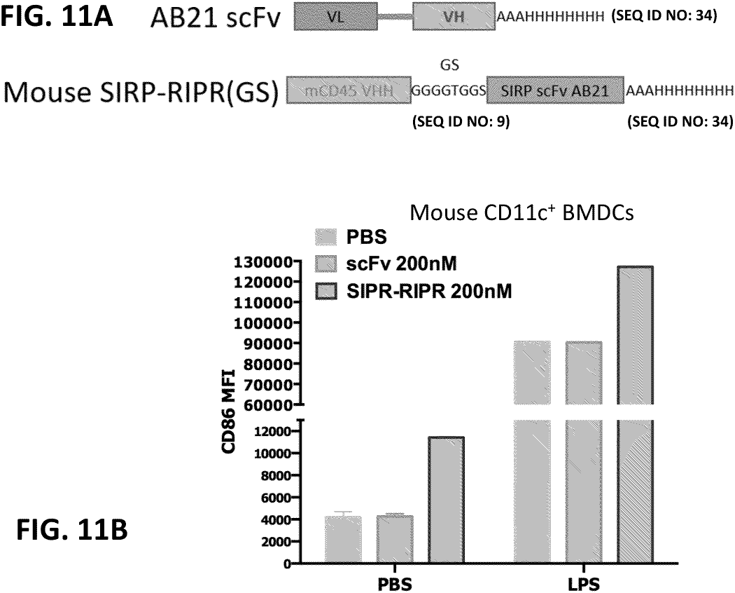

This Examples describes the results of experiments performed to demonstrate that SIRPα-RIPR promotes maturation of bone marrow dendritic cells (BMDCs). In these experiments, murine BMDCs were cultured in complete RPMI1640 medium supplemented with 20 ng/mL GM-CSF. The medium was half changed at Day 3, entirely changed at Day 6. The cells were harvested and were treated with SIRPα-RIPR ligands at Day 7. In these experiments, a bispecific antibody SIRPα-RIPR described in Example 8 and FIGS. 9A-9B was used. As shown in FIG. 11B, the BMDC cells were stimulated with 200 nM AB21 scFv or SIRPα-RIPR for 24 hours at 37° C. CD86 was analyzed on CD11c⁺ population by flow cytometry. A control group treated with lipopolysaccharide (LPS, 1 µg/mL) was also included for control purposes.

CD86 is known to be up-regulated in BMDCs exposed to LPS. CD86 interacts with CD28 present in T cells and potentiates T cell responses. Efficient maturation of BMDCs is correlated with enhanced T cell cytolytic activity. CD86 up-regulation is part of the BMDC maturation process, often also referred to as BMDC “priming.” In these experiments, it was observed that CD86 was also highly upregulated in BMDCs treated with SIRPα-RIPR, indicating that SIRPα-RIPR promotes maturation of the treated BMDCs.

### Example 10. SIRPα-RIPR Enhances the Maturation of Mouse Bone Marrow Dendritic Cells (BMDCs)

This Example describes results of experiments performed to demonstrate that mouse SIRPα-RIPR enhances the maturation of mouse bone marrow dendritic cells (BMDCs). Dendritic cells (DCs) are professional antigen-presenting cells (APCs) whose primary role is to process and present antigens to T lymphocytes to induce adaptive immunity. DCs are heterogeneous. Human DC subtypes include conventional DCs (cDCs), plasmacytoid DCs (pDCs), and monocyte-derived DCs (moDC), which all arise from separate hematopoietic precursors. BMDC and moDC were usually used for studying DC at inflammatory state. pDCs, cDC1 and cDC2 are three subsets of steady-state DC population in both human and mouse.

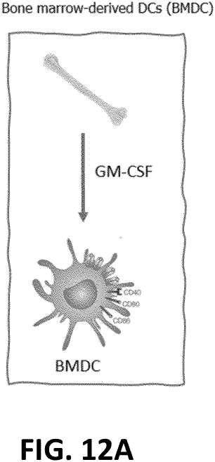

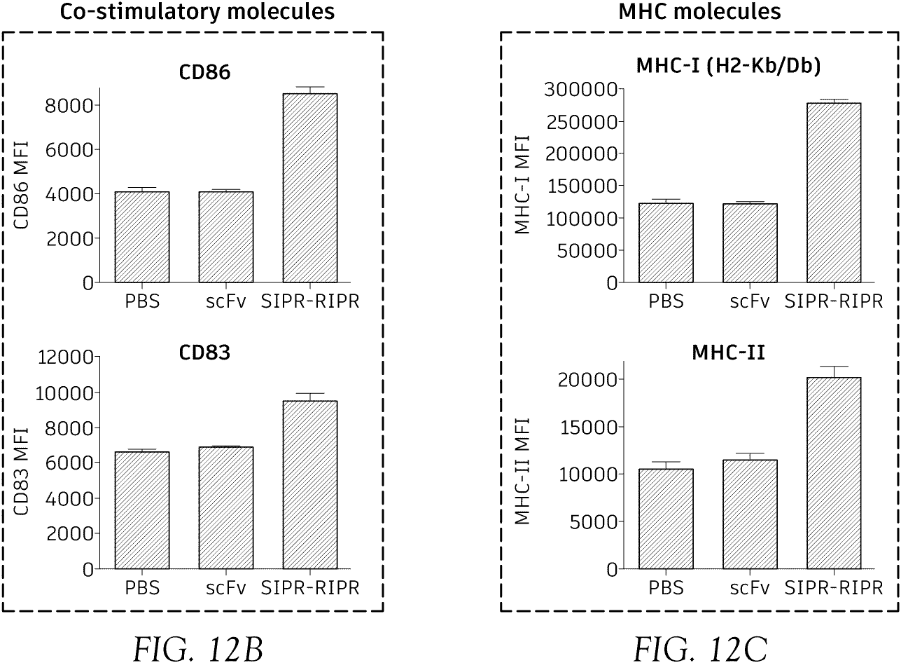

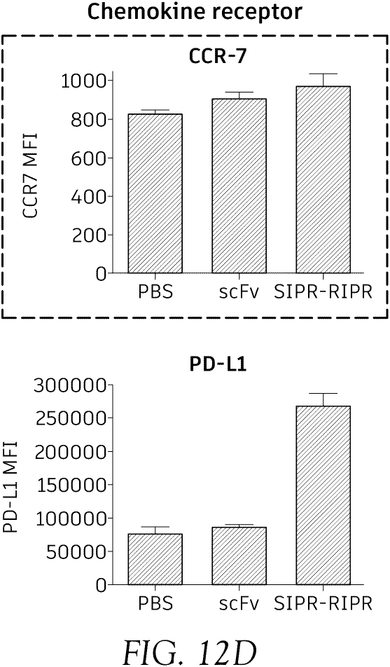

FIG. 12A shows a schematic depiction of BMDC differentiation. Analysis of surface expression of co-stimulatory molecules (CD83, CD86), MHC molecules (MHC-I, MHC-II), chemokine receptor CCR-7 and PD-L1 on CD11c⁺ population are shown in FIGS. 12B-12D. In these experiments, BMDC cells were stimulated with 200 nM AB21 scFv or SIRPα-RIPR for 24 hours at 37° C. Generally, murine BMDCs were cultured in complete RPMI1640 medium supplemented with 20 ng/mL GM-CSF. The medium was half changed at Day 3, entirely changed at Day 6. The cells were harvested and were treated with SIRPα-RIPR ligands at Day 7

Surface expression of co-stimulatory molecules (CD83, CD86), MHC molecules (MHC-I, MHC-II), chemokine receptor CCR-7 and PD-L1 was analyzed on CD11c⁺ population by flow cytometry. Generally, the BMDC cells were stained with antibodies against CD83, CD86, MHC-I, MHC-II, CCR-7 and PD-L1 and measured by FACS, the results were analyzed by FlowJo.

### Example 11. SIRPα-RIPR Potentiates the Maturation of cDC2 Cells

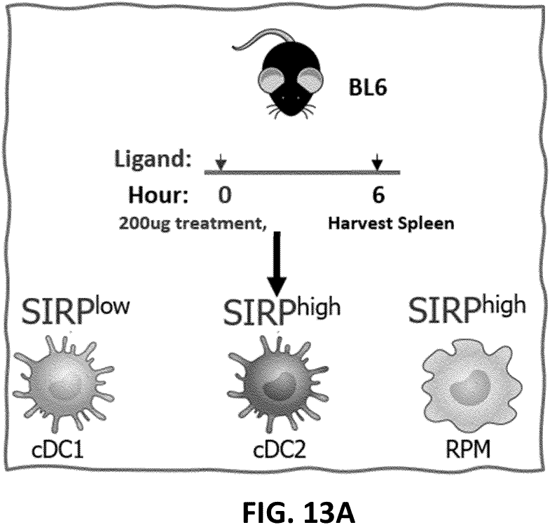

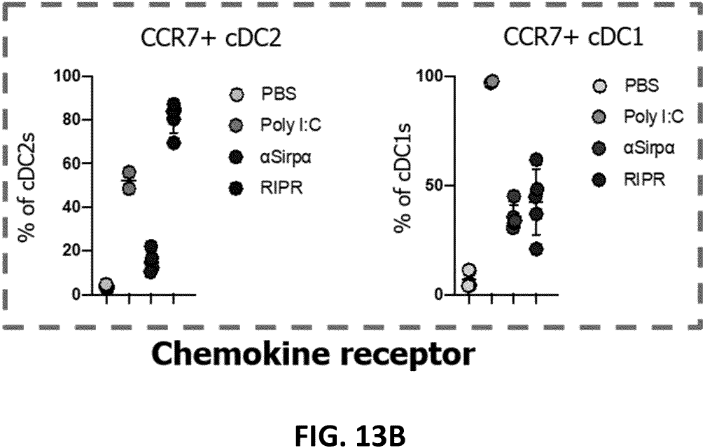

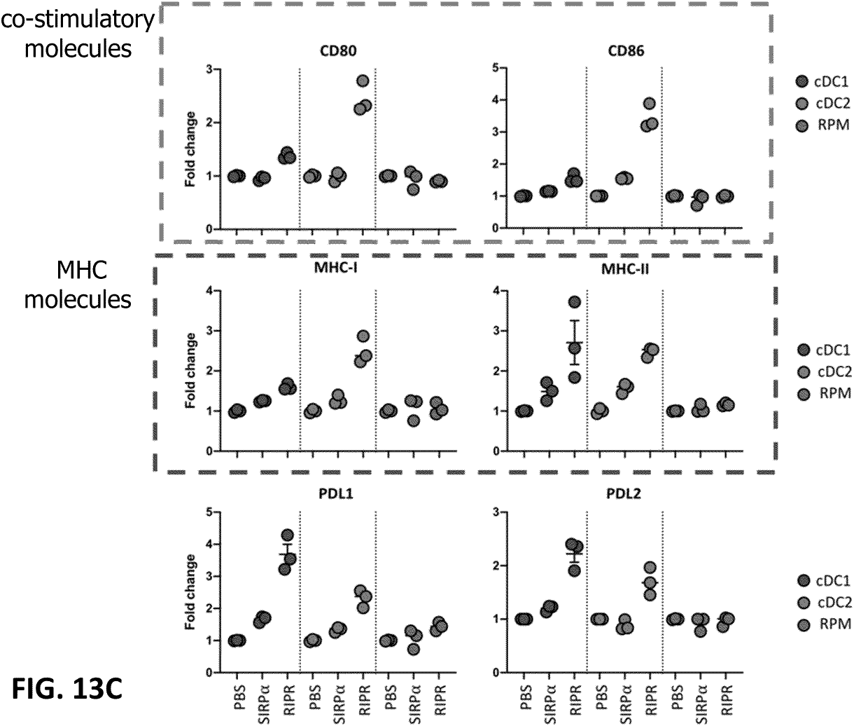

This Example describes results of experiments performed to demonstrate that SIRPα-RIPR potentiates the maturation of cDC2 cells. A general workflow of testing SIRPα-RIPR on conventional dendritic cells and red pulp macrophages (RPM) in C57BL/6 spleen is shown in FIG. 13A. It was observed that cDC2 and RPM expressed much higher level of SIRPα than cDC1. In thes experiments, cDC1 and RPM served as controls for studying cDC2. C57BL/6 mice were intraperitoneally treated with 200 µg AB21 scFv or SIRPα-RIPR for 6 hours. Splenocytes were isolated. Surface expressions of CD80, CD86, MHC-I, MHC-II, CCR-7, PD-L1 and PD-L2 were analyzed on cDC1, cDC2 or RPM by flow cytometry and the results are shown in FIG. 13B-13B. Generally, the isolated splenocytes cells were stained with antibodies against CD83, CD86, MHC-I, MHC-II, CCR-7, PD-L1, CD11b, CD11c, and/or XCR-1. CDC1 cells were gated as CD11c+, CD11b+ XCR1+. CDC2 cells were gated as CD11c+, CD11b+ XCR1-. After measuring by FACS, the results were analyzed by FlowJo.

### Example 12. SIRPα-RIPR Potentiates Proinflammatory Cytokines Production in BMDCs

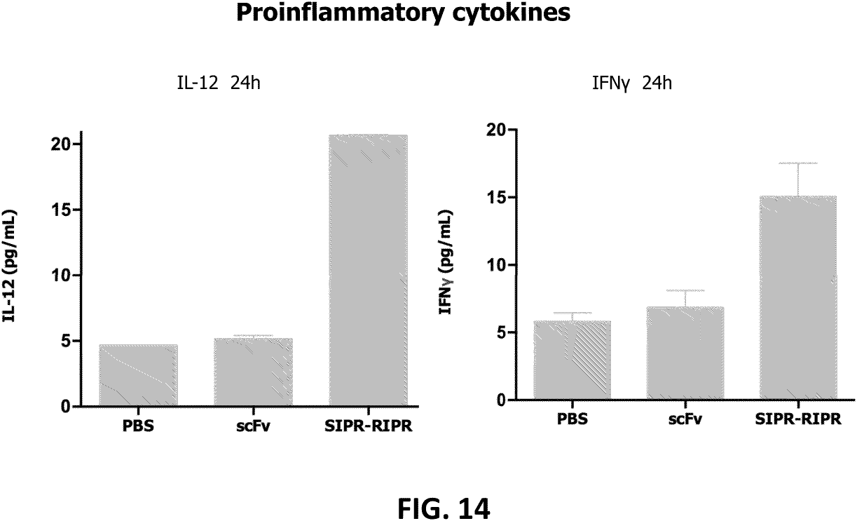

This Example describes results of experiments performed to demonstrate that SIRPα-RIPR potentiates proinflammatory cytokines production in BMDCs. As shown FIG. 14, 100,000 BMDC cells were stimulated with 200 nM AB21 scFv or SIRPα-RIPR for 24 hours at 37° C. IL-12 and IFNγ in supernatant were quantified by ELISA. Generally, murine BMDCs were cultured in complete RPMI1640 medium supplemented with 20 ng/mL GM-CSF. The medium was half changed at Day 3, entirely changed at Day 6. The cultured cells were harvested and were treated with SIRP α—RIPR ligands at Day 7. About 100,000 BMDC cells were seeded in 96 well plates and stimulated with 200 nM AB21 scFv or SIRPα-RIPR for 24 hours at 37° C. After centrifugation at 1600 rpm for 4 minutes, the supernatents were collected. The levels of IL-12, IFNγ in supernatant samples were determined by IL-12 and IFNy ELISA in accordance the manufacturer’s instructions (ELISA kits, BioLegend). The results were measured in a SpectraMax i3x plate reader and plotted in Graphpad Prism.

The experimental results described in this Example indicate that IL-12 is a proinflammatory cytokine that is important for the induction of Th1 cells. Production of IFNγ also reflects the activation of DC cells. In particular, in this study, it was observed that SIRPα-RIPR induces production of IL-12 or IFNy for 3-4 folds higher than AB21. SIRPα-RIPR mediated SIRP silencing results in DC activation.

### Example 13. SIRPα-RIPR Potentiates Cross Presentation of DCs

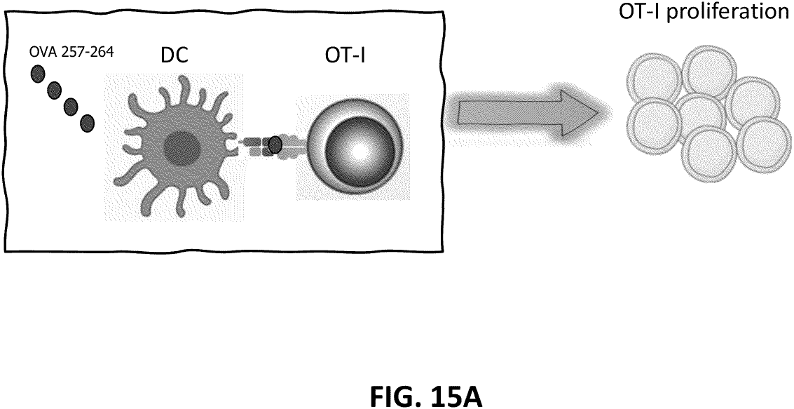

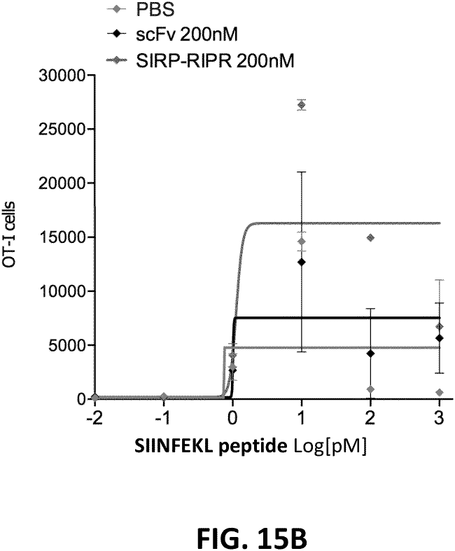

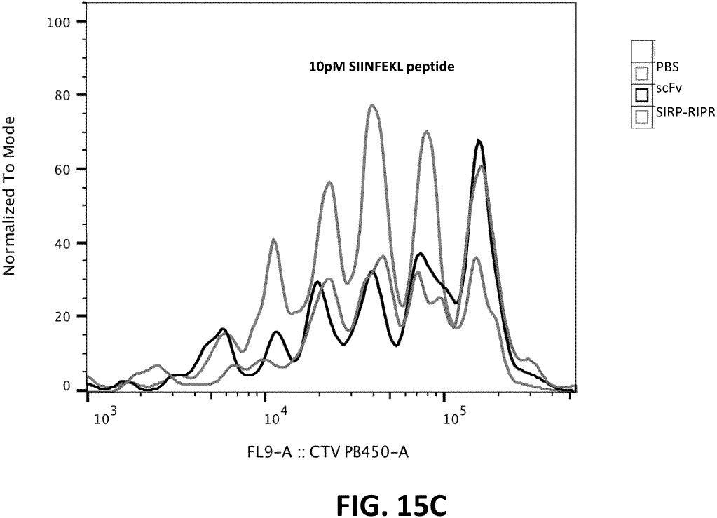

This Example describes results of experiments performed to demonstrate that SIRPα-RIPR potentiates cross presentation of DCs. A schematic depiction of cross presentation of ovalbumin peptide 257-264 (SIINFEKL; SEQ ID NO: 32) is shown in FIG. 15A. The dose response effect of OVA257-264 peptide on OT-I cells proliferation was investigated. OT-I cells were isolated from lymph nodes of OT-I mice and purified by CD8 MACS kit. BMDC cells were pulsed with 10 pM OVA257-264 peptides for 3 hours at 37° C. 50,000 APC cells were co-cultured 1:1 with CTV+ OT-I cells in the presence of 200 nM AB21 scFv or SIRPα-RIPR for 5 days. The dose response effect of OVA257-264 peptide on OT-I cells proliferation were quantified by FACS (FIG. 15B). Dilution of Cell Trace Violet (CTV) in OT-I cells was analyzed by flow cytometry and gated on CD3⁺ CD8⁺ population (FIG. 15C).

IL-12 is produced by DC cells in response to infection by viruses or bacteria pathogens, bridges DC maturation and cytotoxic T cell responses. High level of IL-12 was detected in SIRPα-RIPR treated DCs, which indicates that SIRPα-RIPR might promote antigen- specific cytotoxic T lymphocyte (CTL) responses. Therefore, experiments described in this Example were performed to evaluate CD8+ OT-I T cell proliferative response to ovalbumin (OVA)-pulsed DCs by CFSE labeling in vitro. As expected, both AB21 and SIRPα-RIPR were found to promote the proliferation of CD8+ T cells. Surprisingly, SIRPα-RIPR induces 2-3 folds higher of CD8+ T cells as compared to AB21. Without being bound to any particular theory, these results indicate that SIRPα-RIPR mediated SIRP silencing is potent in enhancing CTL responses, which could be an effective means of inducing antitumor response.

### Example 14. SIRPα-RIPR Potentiates the BMDC Capacity to Induce OT-II Cell Proliferation

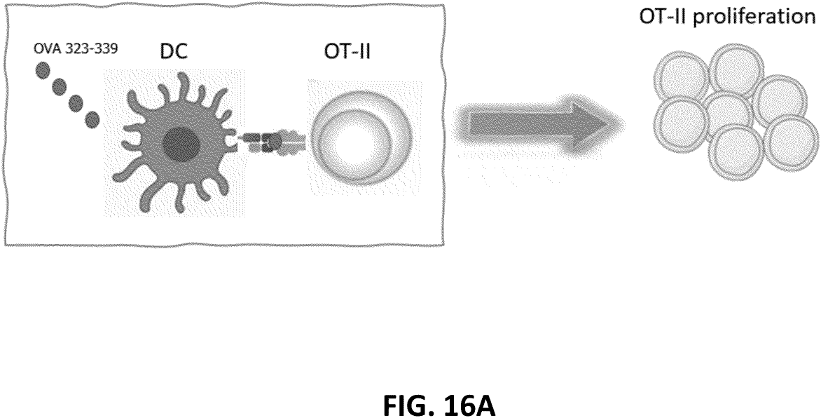

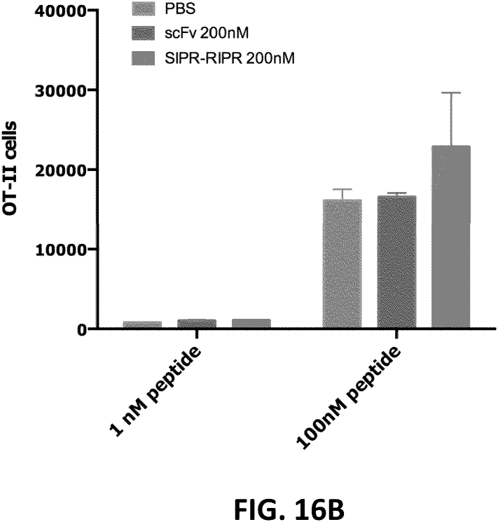

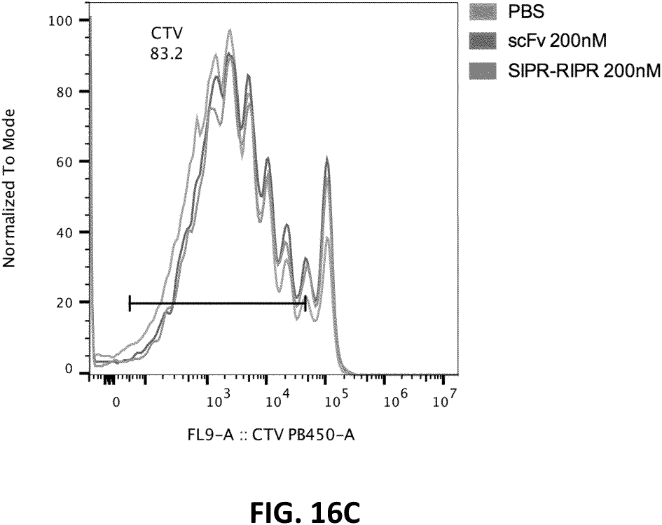

This Example describes results of experiments performed to demonstrate that SIRPα-RIPR potentiates the capacity of BMDCs to induce OT-II cells proliferation. A schematic depiction of antigen presentation of ovalbumin peptide 323-239 (ISQAVHAAHAEINEAGR; SEQ ID NO: 33) is shown in FIG. 16A. The dose response effect of OVA323-339 peptide on OT-II cells proliferation was investigated. OT-II cells were isolated from lymph nodes of OT-II mice. BMDC cells were pulsed with 1 nM or 100 nM ovalbumin (323-339) peptide for 4 hours at 37° C. 50,000 APC cells were co-cultured 1:1 with CTV+ OT-II cells in the presence of 200 nM AB21 scFv or SIRPα-RIPR for 5 days. OT-II cells were counted by FACS (FIG. 16B). Dilution of CTV in OT-II cells was analyzed by flow cytometry and gated on CD3⁺ CD4⁺ population (FIG. 16C).

It was reported that cDC2 is responsible for priming CD4 T cells. Therefore, the enhanced activation of cDC2 by SIRPα-RIPR led to the hypothesis that SIRPα-RIPR might enhance the OT-II T cells activation. Therefore, experiments described in this Example were performed to evaluate the antigen specific CD4+ OT-II proliferation by co-culturing with SIRPα-RIPR or Ab control treated BMDC cells. It was observed that SIRPα-RIPR induces higher level of OT-II proliferation as compared to AB21. This result indicates that SIRP signaling negatively regulated the CD4 T cells priming activity in DCs. Accordingly, reduction of SIRP signaling by SIRPα-RIPR can potentiate the CD4 T cells priming.

### Example 15. SIRPα-RIPR Enhances the BMDC Capacity to Induce Allogeneic T Cell Proliferation

This Example describes results of experiments performed to demonstrate that SIRPα-RIPR enhances the capacity of BMDCs to induce proliferation of allogeneic T cells. In these experiments, an allogeneic mixed lymphocyte reaction (MLR) was performed to evaluate whether silencing of SIRP signaling by SIRPα-RIPR in BMDCs affects interactions with T lymphocytes by assessing the function and proliferative ability of T lymphocytes. T cells could be distinguished by labeling cells in MLR culture with CTV. By analyzing the T cell proliferation, more T cell proliferation was observed in SIRPα-RIPR treatment than AB21.

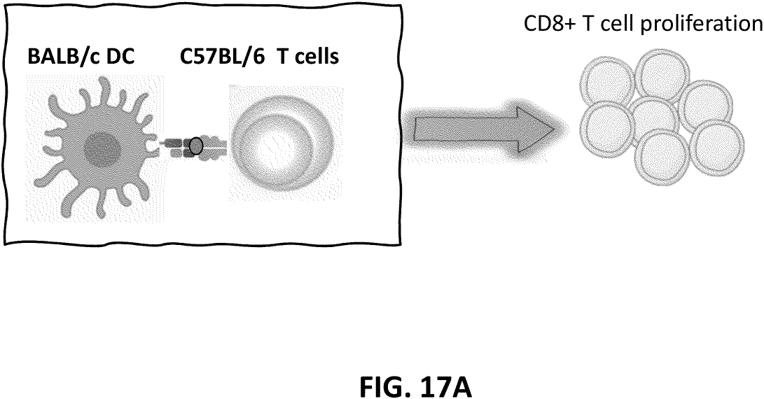

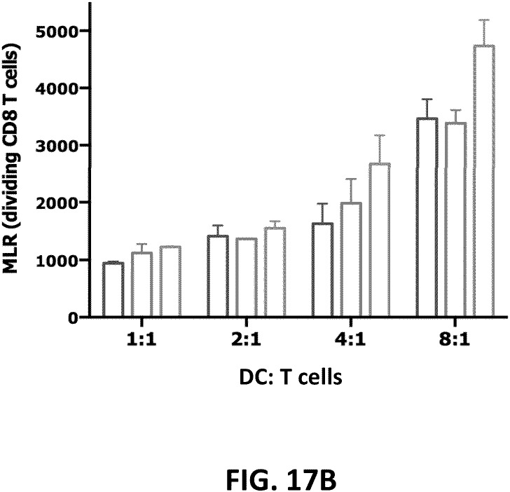

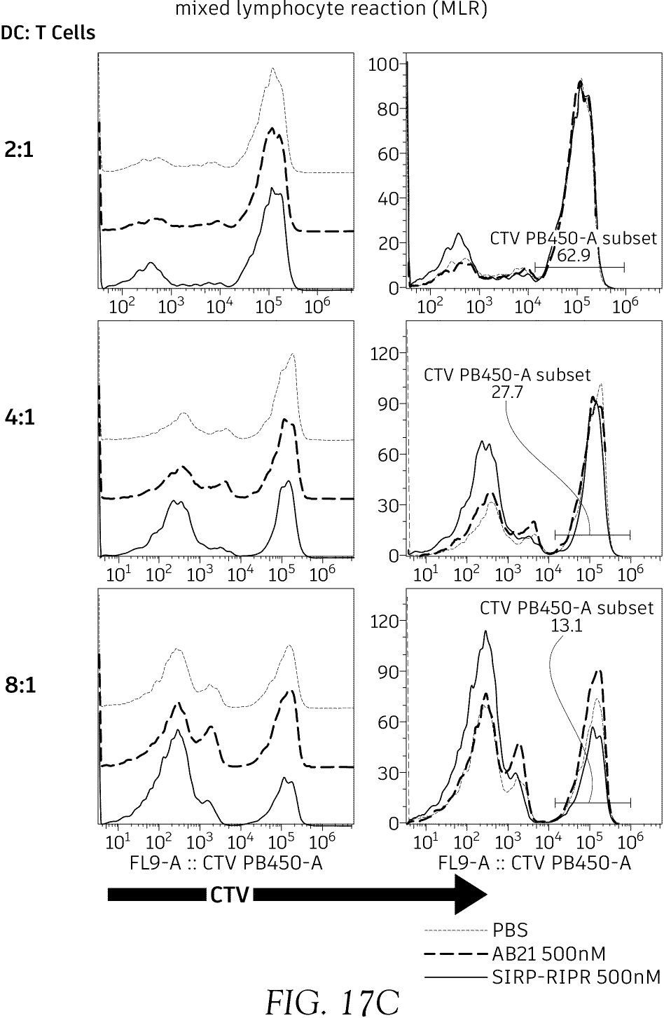

A schematic depiction of mixed-lymphocyte reaction (MLR) is illustrated in FIG. 17A. BMDCs from BALB/c mice were incubated with 50,000 allogeneic spleen T cells from C57BL/6 at different ratios in the presence of 500 nM AB21 scFv or SIRPα-RIPR. CD8+ T cells were counted by FACS. The results of MLR are shown in FIG. 17B. Dilution of CTV in CD8+ T cells was analyzed by flow cytometry and gated on CD3⁺ CD8⁺ population (FIG. 17C).

### Example 16. SIRPα-RIPR Potentiates the Antitumor Response

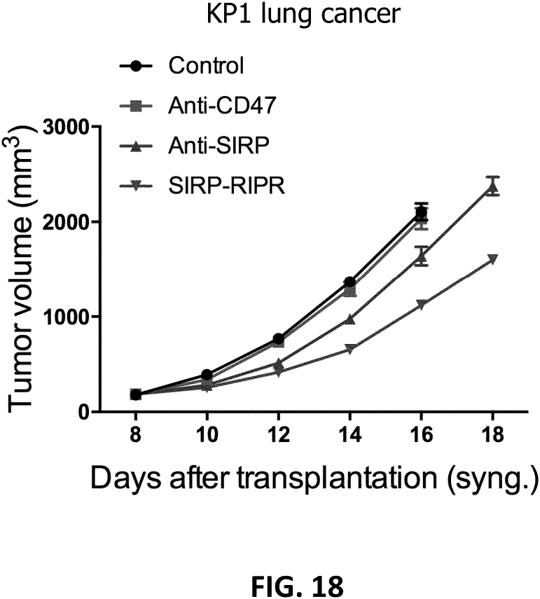

This Example describes results of experiments performed to demonstrate that SIRPα-RIPR potentiates the antitumor response in KP1 lung cancer. F1 mice were implanted with 1×10⁶ KP1 cells and then treated with 200 µg AB21 scFv (n=5), SIRPα-RIPR (n=5) or anti-CD47 (n=5) every other day starting from Day 9. Tumor size was measured starting from Day 8. As shown in FIG. 18, SIRPα-RIPR potentiated the antitumor response in KP1 lung cancer.

### Example 17. SIRPα-RIPR Enhances the Infiltration of Tumor Associated Macrophages

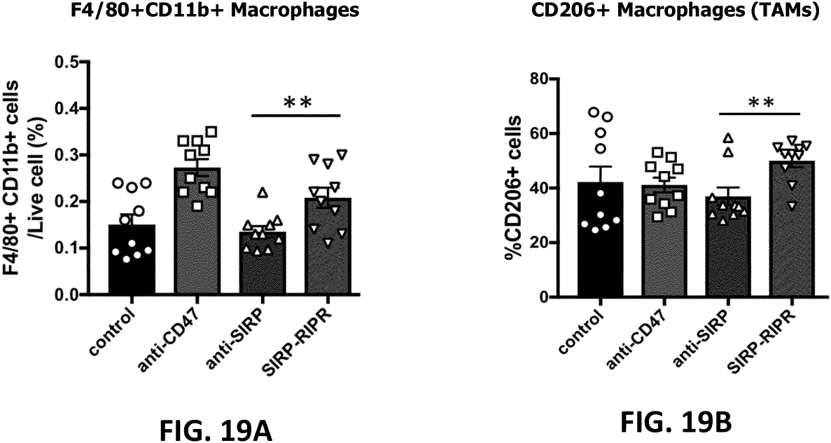

This Example describes results of experiments performed to demonstrate that SIRPα-RIPR enhances the infiltration of tumor associated macrophages in KP1 tumor. Tumor infiltrating lymphocytes were isolated by Ficoll and stained for pan macrophage markers F4/80 and CD11b (n=10) (FIG. 19A). CD206⁺ tumor associated macrophages were quantified by FACS (n=10) (FIG. 19B). In these experiments, the tumors are described in from FIG. 18 at Day 5 post SIRPα-RIPR treatment. For mouse tumors, mouse war first euthanized according to Stanford University-approved protocol, and the tumor area was sprayed with 70% ethanol. The tumor was excised using sterile scissors and forceps. Mouse tumors were excised and minced. The cell suspension was passed through a cell strainer. 30 ml of cell suspension was mixed with 20 ml Ficoll-Paque media, and centrifuged at 1025 x g for 20 min at 20° C. with slow acceleration and brakes turned off. The layer of mononuclear cells was transferred to a sterile tube. The cells were then washed and stained with antibodies against CD45, CD11b, CD206, CD86, CD11c, F4/80, CD19, NK1.1, CD3, CD19 and Live/Dead. After measuring by FACS, the results were analyzed by FlowJo. The experimental results described in FIGS. 19A-19B together illustrate that SIRPα-RIPR enhanced the infiltration of tumor associated macrophages in KP1 tumor.

### Example 18. SIRPα-RIPR Enhances the DC Maturation

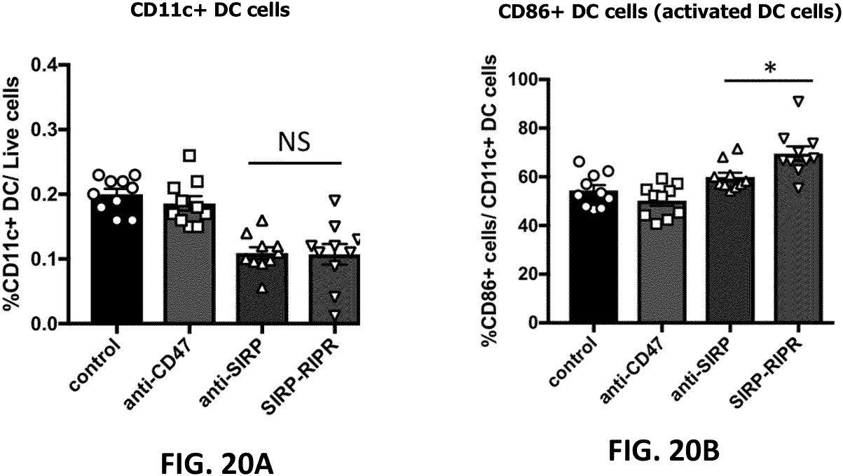

This Example describes results of experiments performed to demonstrate that SIRPα-RIPR enhances the DC maturation in KP1 tumor. Tumor infiltrating lymphocytes were stained for DC marker CD11c (n=10) (FIG. 20A). CD86⁺ DCs were quantified by FACS (n=10) (FIG. 20B).

In these experiments, the tumors are described in from FIG. 18 at Day 5 post SIRPα-RIPR treatment. For mouse tumors, mouse was first euthanized according to Stanford University-approved protocol, and the tumor area was sprayed with 70% ethanol. The tumor was excised using sterile scissors and forceps. Mouse tumors were excised and minced. The cell suspension was passed through a cell strainer. 30 ml of cell suspension was mixed with 20 ml Ficoll-Paque media, and centrifuged at 1025 x g for 20 min at 20° C. with slow acceleration and brakes turned off. The layer of mononuclear cells was transferred to a sterile tube. The cells were then washed and stained with antibodies against CD45, CD11b, CD206, CD86, CD11c, F4/80, CD19, NK1.1, CD3, CD19 and Live/Dead. After measuring by FACS, the results were analyzed by FlowJo. The experimental results described in FIGS. 20A-20B together illustrates that SIRPα-RIPR enhanced the DC maturation in KP1 tumor.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### REFERENCES

1. Wong, H. S. & Germain, R. N. Robust control of the adaptive immune system. Semin. Immunol. 36, 17-27 (2018).

2. Smith-Garvin, J. E., Koretzky, G. A. & Jordan, M. S. T cell activation. Annu. Rev. Immunol. 27, 591-619 (2009).

3. Dushek, O., Goyette, J. & van der Merwe, P. A. Non-catalytic tyrosine-phosphorylated receptors. Immunol. Rev. 250, 258-276 (2012).

4. Lim, W. A. & Pawson, T. Phosphotyrosine signaling: evolving a new cellular communication system. Cell 142, 661-7 (2010).

5. van der Merwe, P. A. & Dushek, O. Mechanisms for T cell receptor triggering. Nat. Rev. Immunol. 11, 47-55 (2011).

6. Pingel, J. T. & Thomas, M. L. Evidence that the leukocyte-common antigen is required for antigen-induced T lymphocyte proliferation. Cell 58, 1055-65 (1989).

7. Freeman, S. A. et al. Integrins Form an Expanding Diffusional Barrier that Coordinates Phagocytosis. Cell 164, 128-140 (2016).

8. Hermiston, M. L., Xu, Z. & Weiss, A. CD45: a critical regulator of signaling thresholds in immune cells. Annu. Rev. Immunol. 21, 107-37 (2003).

9. Tonks, N. K. Protein tyrosine phosphatases: From genes, to function, to disease. Nature Reviews Molecular Cell Biology 7, 833-846 (2006).

10. Barr, A. J. et al. Large-Scale Structural Analysis of the Classical Human Protein Tyrosine Phosphatome. Cell 136, 352-363 (2009).

11. Myers, D. R., Zikherman, J. & Roose, J. P. Tonic Signals: Why Do Lymphocytes Bother? Trends in Immunology 38, 844-857 (2017).

12. Mahtani-Patching, J. et al. PreTCR and TCR{gamma} {delta} Signal Initiation in Thymocyte Progenitors Does Not Require Domains Implicated in Receptor Oligomerization. Sci. Signal. 4, ra47 (2011).

13. Irving, B. A., Alt, F. W. & Killeen, N. Thymocyte development in the absence of pre-T cell receptor extracellular immunoglobulin domains. Science 280, 905-8 (1998).

14. Chang, V. T. et al. Initiation of T cell signaling by CD45 segregation at ‘close contacts’. Nat. Immunol. 17, 574-82 (2016).

15. Stefanová, I., Dorfman, J. R. & Germain, R. N. Self-recognition promotes the foreign antigen sensitivity of naive T lymphocytes. Nature 420, 429-434 (2002).

16. Delon, J., Bercovici, N., Raposo, G., Liblau, R. & Trautmann, A. Antigen-dependent and -independent Ca2+ responses triggered in T cells by dendritic cells compared with B cells. J. Exp. Med. 188, 1473-84 (1998).

17. Monroe, J. G. ITAM-mediated tonic signalling through pre-BCR and BCR complexes. Nat. Rev. Immunol. 6, 283-294 (2006).

18. Bezbradica, J. S., Rosenstein, R. K., Demarco, R. A., Brodsky, I. & Medzhitov, R. A role for the ITAM signaling module in specifying cytokine-receptor functions. Nat. Immunol. 15, 333-342 (2014).

19. Shah, K. M. et al. The EBV-encoded latent membrane proteins, LMP2A and LMP2B, limit the actions of interferon by targeting interferon receptors for degradation. Oncogene 28, 3903-14 (2009).

20. Lee, B.-S., Connole, M., Tang, Z., Harris, N. L. & Jung, J. U. Structural analysis of the Kaposi’s sarcoma-associated herpesvirus K1 protein. J. Virol. 77, 8072-86 (2003).

21. Bian, Z. et al. Cd47-Sirpα interaction and IL-10 constrain inflammation-induced macrophage phagocytosis of healthy self-cells. Proc. Natl. Acad. Sci. U. S. A. 113, E5434-43 (2016).

22. Vijayakrishnan, L. et al. An Autoimmune Disease-Associated CTLA-4 Splice Variant Lacking the B7 Binding Domain Signals Negatively in T Cells. Immunity 20, 563-575 (2004).

23. Hanawa, H. et al. A novel costimulatory signaling in human T lymphocytes by a splice variant of CD28. Blood 99, 2138-45 (2002).

24. Wei, F. et al. Strength of PD-1 signaling differentially affects T-cell effector functions. Proc. Natl. Acad. Sci. 110, E2480-E2489 (2013).

25. Wang, S.-F. et al. Early T Cell Signalling Is Reversibly Altered in PD-1+ T Lymphocytes Infiltrating Human Tumors. PLoS One 6, e17621 (2011).

26. Bardhan, K. et al. Phosphorylation of PD-1-Y248 is a marker of PD-1-mediated inhibitory function in human T cells. Sci. Rep. 9, (2019).

27. Baumeister, S. H., Freeman, G. J., Dranoff, G. & Sharpe, A. H. Coinhibitory Pathways in Immunotherapy for Cancer. Annu. Rev. Immunol. 34, 539-573 (2016).

28. Fritz, J. M. & Lenardo, M. J. Development of immune checkpoint therapy for cancer. J. Exp. Med. 216, 1244-1254 (2019).

29. Okazaki, T. & Honjo, T. PD-1 and PD-1 ligands: from discovery to clinical application. Int. Immunol. 19, 813-824 (2007).

30. Francisco, L. M., Sage, P. T. & Sharpe, A. H. The PD-1 Pathway in Tolerance and Autoimmunity. Immunol. Rev. 236, 219 (2010).

31. Riley, J. L. PD-1 signaling in primary T cells. Immunol. Rev. 229, 114-25 (2009).

32. Chemnitz, J. M., Parry, R. V, Nichols, K. E., June, C. H. & Riley, J. L. SHP-1 and SHP-2 associate with immunoreceptor tyrosine-based switch motif of programmed death 1 upon primary human T cell stimulation, but only receptor ligation prevents T cell activation. J. Immunol. 173, 945-54 (2004).

33. Riley, J. L. & June, C. H. The CD28 family: A T-cell rheostat for therapeutic control of T-cell activation. Blood 105, 13-21 (2005).

34. Mizuno, R. et al. PD-1 Primarily Targets TCR Signal in the Inhibition of Functional T Cell Activation. Front. Immunol. 10, 630 (2019).

35. Yokosuka, T. et al. Programmed cell death 1 forms negative costimulatory microclusters that directly inhibit T cell receptor signaling by recruiting phosphatase SHP2. J. Exp. Med. 209, 1201-1217 (2012).

36. Hamid, O. et al. Safety and Tumor Responses with Lambrolizumab (Anti-PD-1) in Melanoma. N. Engl. J. Med. 369, 134-144 (2013).

37. Ribas, A. et al. Association of Pembrolizumab With Tumor Response and Survival Among Patients With Advanced Melanoma. JAMA 315, 1600 (2016).

38. Gong, J., Chehrazi-Raffle, A., Reddi, S. & Salgia, R. Development of PD-1 and PD-L1 inhibitors as a form of cancer immunotherapy: a comprehensive review of registration trials and future considerations. J. Immunother. cancer 6, 8 (2018).

39. Brahmer, J. et al. Nivolumab versus Docetaxel in Advanced Squamous-Cell Non-Small-Cell Lung Cancer. N. Engl. J. Med. 373, 123-135 (2015).

40. Marasco, M. et al. Molecular mechanism of SHP2 activation by PD-1 stimulation. Sci. Adv. 6, eaay4458 (2020).

41. Beane, J. D. et al. Clinical Scale Zinc Finger Nuclease-mediated Gene Editing of PD-1 in Tumor Infiltrating Lymphocytes for the Treatment of Metastatic Melanoma. Mol. Ther. 23, 1380-1390 (2015).

42. Nam, H.-J., Poy, F., Saito, H. & Frederick, C. A. Structural basis for the function and regulation of the receptor protein tyrosine phosphatase CD45. J. Exp. Med. 201, 441-52 (2005).

43. O’Shea, J. J., McVicar, D. W., Bailey, T. L., Burns, C. & Smyth, M. J. Activation of human peripheral blood T lymphocytes by pharmacological induction of protein-tyrosine phosphorylation. Proc. Natl. Acad. Sci. U.S.A. 89, 10306-10 (1992).

44. Hui, E. et al. T cell costimulatory receptor CD28 is a primary target for PD-1-mediated inhibition. Science (80-.). 355, 1428-1433 (2017).

45. Wang, C. et al. In vitro characterization of the anti-PD-1 antibody nivolumab, BMS-936558, and in vivo toxicology in non-human primates. Cancer Immunol. Res. 2, 846-856 (2014).

46. Selby, M. J. et al. Preclinical Development of Ipilimumab and Nivolumab Combination Immunotherapy: Mouse Tumor Models, In Vitro Functional Studies, and Cynomolgus Macaque Toxicology. PLoS One 11, e0161779 (2016).

47. Long, A. H. et al. 4-1BB costimulation ameliorates T cell exhaustion induced by tonic signaling of chimeric antigen receptors. Nat. Med. 21, 581-590 (2015).

48. Neal, J. T., Li, X., Zheng, G. X. Y., Davis, M. M. & Kuo Correspondence, C. J. Organoid Modeling of the Tumor Immune Microenvironment. Cell 175, (2018).

49. Bunn, P. A. et al. Small Cell Lung Cancer: Can Recent Advances in Biology and Molecular Biology Be Translated into Improved Outcomes? J. Thorac. Oncol. 11, 453-474 (2016).

50. Sabari, J. K., Lok, B. H., Laird, J. H., Poirier, J. T. & Rudin, C. M. Unravelling the biology of SCLC: implications for therapy. Nat. Rev. Clin. Oncol. 14, 549-561 (2017).

51. Hellmann, M. D. et al. Tumor Mutational Burden and Efficacy of Nivolumab Monotherapy and in Combination with Ipilimumab in Small-Cell Lung Cancer. Cancer Cell 33, 853-861.e4 (2018).

52. Zimmerman, S. et al. 2017-2018 Scientific Advances in Thoracic Oncology: Small Cell Lung Cancer. J. Thorac. Oncol. 14, 768-783 (2019).

53. Horn, L. et al. First-Line Atezolizumab plus Chemotherapy in Extensive-Stage Small-Cell Lung Cancer. N. Engl. J. Med. 379, 2220-2229 (2018).

54. Meuwissen, R. et al. Induction of small cell lung cancer by somatic inactivation of both Trp53 and Rb1 in a conditional mouse model. Cancer Cell 4, 181-9 (2003).

55. George, J. et al. Comprehensive genomic profiles of small cell lung cancer. Nature 524, 47-53 (2015).

56. Gattinoni, L. et al. Wnt signaling arrests effector T cell differentiation and generates CD8+ memory stem cells. Nat. Med. 15, 808-13 (2009).

57. Schilham, M. W. et al. Critical involvement of Tcf-1 in expansion of thymocytes. J. Immunol. 161, 3984-91 (1998).

58. Woo, S. R. et al. Immune inhibitory molecules LAG-3 and PD-1 synergistically regulate T-cell function to promote tumoral immune escape. Cancer Res. 72, 917-927 (2012).

59. Khan, O. et al. TOX transcriptionally and epigenetically programs CD8+ T cell exhaustion. Nature 571, 211-218 (2019).

60. Scott, A. C. et al. TOX is a critical regulator of tumour-specific T cell differentiation. Nature 571, 270-274 (2019).

61. Weiskopf, K. et al. Engineered SIRPα variants as immunotherapeutic adjuvants to anticancer antibodies. Science (80-.). 341, 88-91 (2013).

62. Ho, C. C. M. et al. ‘Velcro’ engineering of high affinity CD47 Ectodomain as signal regulatory protein α (SIRPα) antagonists that enhance antibody-dependent cellular phagocytosis. J. Biol. Chem. 290, 12650-12663 (2015).

63. Davis, S. J. & van der Merwe, P. A. The kinetic-segregation model: TCR triggering and beyond. Nat. Immunol. 7, 803-9 (2006).

64. Rossotti, M. et al. Streamlined method for parallel identification of single domain antibodies to membrane receptors on whole cells. Biochim. Biophys. Acta - Gen. Subj. 1850, 1397-1404 (2015).

65. Zhao, P. et al. Depletion of PD-1-positive cells ameliorates autoimmune disease. Nat. Biomed. Eng. 3, 292-305 (2019).

66. Ingram, J. R. et al. PD-L1 is an activation-independent marker of brown adipocytes. Nat. Commun. 8, (2017).

67. Dumoulin, M. et al. A camelid antibody fragment inhibits the formation of amyloid fibrils by human lysozyme. Nature 424, 783-788 (2003).

68. Dumoulin, M. et al. Single-domain antibody fragments with high conformational stability. Protein Sci. 11, 500-515 (2009).

69. Liu Q. et al. Inhibition of SIRPα in dendritic cells potentiates potent antitumor immunity. Oncoimmunology. 2016 Aug 23;5(9):e1183850. doi: 10.1080/2162402X.2016.1183850.

70. Sim J. et al., Discovery of high affinity, pan-allelic, and pan-mammalian reactive antibodiesagainst the myeloid checkpoint receptor SIRPα. MAbs. 2019, Vol. No. 6, pp. 1036-1052.

What is claimed is:

1. A multivalent polypeptide comprising:
a first amino acid sequence comprising a first polypeptide module capable of binding to a signal regulatory protein α (SIRPα); and
a second amino acid sequence comprising a second polypeptide module capable of binding to one or more receptor protein-tyrosine phosphatases (RPTP) of R1/R6 subfamily.

2. The multivalent polypeptide of a claim 1, wherein the one or more RPTPs comprises CD45 or a functional variant thereof.

3. The multivalent polypeptide of any one of claims 1-2, wherein at least one of the first and second polypeptide modules comprises an amino acid sequence for a protein-binding ligand or an antigen-binding moiety.

4. The multivalent polypeptide of claim 3, wherein the antigen-binding moiety is selected from the group consisting of a single-chain variable fragment (scFv), an antigen-binding fragment (Fab), a nanobody, a V_{H} domain, a V_{L} domain, a single domain antibody (dAb), a V_{NAR} domain, and a V_{H}H domain, a diabody, or a functional fragment of any thereof.

5. The multivalent polypeptide of claim 3, wherein the protein-binding ligand comprises an extracellular domain (ECD) of a cell surface receptor, or an ECD of a RPTP, or a functional variant of any thereof.

6. The multivalent polypeptide of claim 5, wherein the protein-binding ligand comprises an ECD of CD47 or a functional variant thereof.

7. The multivalent polypeptide of any one of claims 1-6, wherein the first polypeptide module is operably linked to the second polypeptide module via a polypeptide linker sequence.

8. The multivalent polypeptide of claim 7, wherein the polypeptide linker sequence comprises a glycine-serine (GS) linker or a 3C linker.

9. The multivalent polypeptide of any one of claims 1-8, comprising:
(a) (i) a CD47 ECD, (ii) a polypeptide linker, and (iii) a CD45 scFv;
(b) (i) a SIRPα scFv, (ii) a polypeptide linker; and (iii) a CD45 scFv; or
(c) (i) a CD45 V_{H}H, (ii) a polypeptide linker, and (iii) a SIRPα scFv.

10. The multivalent polypeptide of claim 9, comprising, in N-terminus to C-terminus direction:
(a) (i) a CD47 ECD, (ii) a GS linker, and (iii) a CD45 scFv; or
(b) (i) a CD47 ECD, (ii) a C3 linker, and (iii) a CD45 scFv.

11. The multivalent polypeptide of claim 9, comprising, in N-terminus to C-terminus direction:
(a) (i) a SIRPα scFv, (ii) a GS linker, and (iii) a CD45 scFv; or
(b) (i) a SIRPα scFv, (ii) a C3 linker, and (iii) a CD45 scFv.

12. The multivalent polypeptide of claim 9, comprising, in N-terminus to C-terminus direction:
(a) (i) a CD45 V_{H}H, (ii) a GS linker, and (iii) a SIRPα scFv; or
(b) (i) a CD45 V_{H}H, (ii) a C3 linker, and (iii) a SIRPα scFv.

13. The multivalent polypeptide of any one of claims 1-12, wherein the multivalent polypeptide comprises an amino acid sequence that has at least 80% sequence identity to an amino acid sequence selected from the group consisting of SEQ ID NOS: 1-6.

14. A recombinant nucleic acid molecule comprising a nucleotide sequence encoding a multivalent polypeptide according to any one of claims 1-13.

15. The recombinant nucleic acid molecule of claim 14, wherein the nucleotide sequence encodes an amino acid sequence having at least 80% sequence identity to an amino acid sequence selected from the group consisting of SEQ ID NOS: 1-6.

16. A recombinant cell comprising:
(a) a multivalent polypeptide according to any one of claims 1-13, and/or
(b) a recombinant nucleic acid molecule of any one of claims 14-15.

17. The recombinant cell of claim 16, wherein the recombinant cell is a phagocytic cell.

18. The recombinant cell of claim 17, wherein the phagocytic cell is a dendritic cell.

19. A method for promoting the maturation of immature dendritic cells (DCs) in vitro, the method comprising:
(a) exposing immature DCs to an antigen; and
(b) culturing the immature DCs in the presence of a multivalent polypeptide according to any one of claims 1-13 to induce the maturation of immature DCs into mature DCs.

20. A mature dendritic cell prepared by the method of claim 19.

21. A method for manufacturing a vaccine, the method comprising:
(a) exposing immature DCs to an antigen in vitro to produce a sufficient number of antigen-presenting immature DCs; and
(b) promoting the maturation of the antigen-presenting immature DCs in the presence of a multivalent polypeptide according to any one of claims 1-13 to produce mature antigen-presenting DCs.

22. A vaccine manufactured by the method of claim 21.

23. The vaccine of claim 22, further comprises a diluent, an excipient, an auxiliary adjuvant, a bacterial adjuvant, and/or a systemic adjuvant.

24. A pharmaceutical composition comprising a pharmaceutical acceptable excipient, and:
(a) a multivalent polypeptide according to any one of claims 1-13;
(b) a recombinant nucleic acid molecule according to any one of claims 14-15; and/or
(c) a recombinant cell according to any one of claims 16-17;
(d) a mature dendritic cell according to claim 20; and /or
(e) a vaccine according to any one of claims 22-23.

25. A method for modulating cell signaling mediated by CD47 and/or SIRPα in a subject, the method comprising administering to the subject a composition comprising:
(a) a multivalent polypeptide according to any one of claims 1-13;
(b) a recombinant nucleic acid molecule according to any one of claims 14-15;
(c) a recombinant cell according to any one of claims 16-17;
(d) a mature dendritic cell according to claim 20;
(e) a vaccine according to any one of claims 22-23; and/or
(f) a pharmaceutical composition of claim 24.

26. A method for preventing or treating a health condition in a subject in need thereof, the method comprising administering to the subject a composition comprising:
(a) a multivalent polypeptide according to any one of claims 1-13;
(b) a recombinant nucleic acid molecule according to any one of claims 14-15;
(c) a recombinant cell according to any one of claims 16-17;
(d) a mature dendritic cell according to claim 20;
(e) a vaccine according to any one of claims 22-23; and/or
(f) a pharmaceutical composition of claim 24.

27. The method of any one of claims 25-26, wherein the administered composition recruits the RPTP activity to a spatial proximity of SIRPα, potentiates dephosphorylation of SIRPα, reduces SIRPα-mediated signaling, promotes dendritic cell maturation, and/or promotes macrophage phagocytosis.

28. The method of any one of claims 25-27, wherein the administered composition confers an enhancement in macrophage-mediated phagocytosis.

29. The method of any one of claims 25-28, wherein the subject has or is suspected of having a health condition associated with CD47 and/or SIRPα.

30. The method of any one of claims 25-29, wherein the health condition is a cancer or a chronic infection.

31. A method for preventing or treating a cancer in a subject in need thereof, the method comprising:
culturing immature DCs to an antigen in vitro with a cancer-associated antigen or infection-associated antigen to produce antigen-presenting immature DCs;
promoting the maturation of antigen-presenting immature DCs in the presence of a multivalent polypeptide according to any one of claims 1-13 to produce mature antigen-presenting DCs; and
administering the subject with the produced mature antigen-presenting DCs.

32. A method of preventing or treating a subject infected or suspected of being suspected with a parasite, a virus, a microfungus, or a bacterium, the method comprising:
culturing immature DCs with an antigen derived from a parasite, a virus, a microfungus, or a bacterium to produce antigen-presenting dendritic cells;
promoting the maturation of dendritic cells in the presence of a multivalent polypeptide according to any one of claims 1-13 to produce mature antigen-presenting DCs; and
administering the subject with the produced mature antigen-presenting DCs.

33. The method of any one of claims 25-32, wherein the subject is a mammalian subject.

34. The method of claim 33, the mammalian subject is a human.

35. The method of any one of claim 26-34, the composition is administered to the subject individually or as a first therapy in combination with a second therapy.

36. The method of claim 35, wherein the second therapy is selected from the group consisting of chemotherapy, radiotherapy, immunotherapy, hormonal therapy, toxin therapy, or surgery further comprising administering to the subject a second therapy.

37. A kit for modulating cell signaling in a subject or for preventing or for treating a health condition in a subject in need thereof, the kit comprising instructions for use thereof and one or more of the following:
(a) a multivalent polypeptide according to any one of claims 1-13;
(b) a recombinant nucleic acid molecule according to any one of claims 14-15;
(c) a recombinant cell according to any one of claims 16-17;
(d) a mature dendritic cell according to claim 20;
(e) a vaccine according to any one of claims 22-23; and
(f) a pharmaceutical composition of claim 24.


SEQUENCE LISTING (34 sequences)

SEQ ID NO: 1 - protein, 387 aa, Artificial sequence
  Synthetic construct
  MISC_FEATURE: Velcro SIRP-RIPR(GS)
WQPPLLFNKTKSVEFTFGNDTVVIPCFVTNMEAQNTTEVYVKWKFKGRDIYTFDGQANKSTVPTDFSSAKIEVSQLLKGDASLKMDKSDAVSHTGNYTCEVTELTREGETIIELKYRVVSWGGGGTGGSEVQLQVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGSTRSSSSGGGGSGGGGDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKAAAHHHHHHHH

SEQ ID NO: 2 - protein, 395 aa, Artificial sequence
  Synthetic construct
  MISC_FEATURE: Velcro SIRP-RIPR(3C)
WQPPLLFNKTKSVEFTFGNDTVVIPCFVTNMEAQNTTEVYVKWKFKGRDIYTFDGQANKSTVPTDFSSAKIEVSQLLKGDASLKMDKSDAVSHTGNYTCEVTELTREGETIIELKYRVVSWGGGSLEVLFQGPGSGSEVQLQVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGSTRSSSSGGGGSGGGGDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKAAAHHHHHHHH

SEQ ID NO: 3 - protein, 509 aa, Artificial sequence
  Synthetic construct
  MISC_FEATURE: Human SIRP-RIPR(GS)
ALTQPASVSANPGETVKIACSGGDYYSYYYGWYQQKAPGSALVTVIYSDDKRPSDIPSRFSGSASGSTATLTITGVRAEDEAVYYCGGYDYSTYANAFGAGTTLTVLGGSTRSSSSGGGGSGGGGDVQLVESGGGVVRPGESLRLSCAASGFTFSSNAMSWVRQAPGKGLEWLAGISAGGSDTYYPASVKGRFTISRDNSKNTLYLQMNTLTAEDTAVYYCARETWNHLFDYWGLGTLVTVSSGGGGTGGSEVQLQVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGSTRSSSSGGGGSGGGGDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKAAAHHHHHHHH

SEQ ID NO: 4 - protein, 509 aa, Artificial sequence
  Synthetic construct
  MISC_FEATURE: Human SIRP-RIPR(3C)
ALTQPASVSANPGETVKIACSGGDYYSYYYGWYQQKAPGSALVTVIYSDDKRPSDIPSRFSGSASGSTATLTITGVRAEDEAVYYCGGYDYSTYANAFGAGTTLTVLGGSTRSSSSGGGGSGGGGDVQLVESGGGVVRPGESLRLSCAASGFTFSSNAMSWVRQAPGKGLEWLAGISAGGSDTYYPASVKGRFTISRDNSKNTLYLQMNTLTAEDTAVYYCARETWNHLFDYWGLGTLVTVSSLEVLFQGPEVQLQVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGSTRSSSSGGGGSGGGGDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKAAAHHHHHHHH

SEQ ID NO: 5 - protein, 381 aa, Artificial sequence
  Synthetic construct
  MISC_FEATURE: Murine SIRP-RIPR(GS)
QVQLVESGGGLVHPGDSLRLSCAASGSVFNSATMGWYRQSPGSQRELVATIVVGTPTYADSVKGRFTISRDNAKNIVYLQMNSLKPEDTAVYYCNYRATYTSGYSRDYWGQGTQVTVSSGGGGTGGSALTQPASVSANPGETVKIACSGGDYYSYYYGWYQQKAPGSALVTVIYSDDKRPSDIPSRFSGSASGSTATLTITGVRAEDEAVYYCGGYDYSTYANAFGAGTTLTVLGGSTRSSSSGGGGSGGGGDVQLVESGGGVVRPGESLRLSCAASGFTFSSNAMSWVRQAPGKGLEWLAGISAGGSDTYYPASVKGRFTISRDNSKNTLYLQMNTLTAEDTAVYYCARETWNHLFDYWGLGTLVTVSSAAAHHHHHHHH

SEQ ID NO: 6 - protein, 381 aa, Artificial sequence
  Synthetic construct
  MISC_FEATURE: Murine SIRP-RIPR(3C)
QVQLVESGGGLVHPGDSLRLSCAASGSVFNSATMGWYRQSPGSQRELVATIVVGTPTYADSVKGRFTISRDNAKNIVYLQMNSLKPEDTAVYYCNYRATYTSGYSRDYWGQGTQVTVSSLEVLFQGPALTQPASVSANPGETVKIACSGGDYYSYYYGWYQQKAPGSALVTVIYSDDKRPSDIPSRFSGSASGSTATLTITGVRAEDEAVYYCGGYDYSTYANAFGAGTTLTVLGGSTRSSSSGGGGSGGGGDVQLVESGGGVVRPGESLRLSCAASGFTFSSNAMSWVRQAPGKGLEWLAGISAGGSDTYYPASVKGRFTISRDNSKNTLYLQMNTLTAEDTAVYYCARETWNHLFDYWGLGTLVTVSSAAAHHHHHHHH

SEQ ID NO: 7 - protein, 132 aa, Artificial sequence
  Synthetic construct
  MISC_FEATURE: Velcro
WQPPLLFNKTKSVEFTFGNDTVVIPCFVTNMEAQNTTEVYVKWKFKGRDIYTFDGQANKSTVPTDFSSAKIEVSQLLKGDASLKMDKSDAVSHTGNYTCEVTELTREGETIIELKYRVVSWAAAHHHHHHHH

SEQ ID NO: 8 - protein, 254 aa, Artificial sequence
  Synthetic construct
  MISC_FEATURE: AB21 scFv
ALTQPASVSANPGETVKIACSGGDYYSYYYGWYQQKAPGSALVTVIYSDDKRPSDIPSRFSGSASGSTATLTITGVRAEDEAVYYCGGYDYSTYANAFGAGTTLTVLGGSTRSSSSGGGGSGGGGDVQLVESGGGVVRPGESLRLSCAASGFTFSSNAMSWVRQAPGKGLEWLAGISAGGSDTYYPASVKGRFTISRDNSKNTLYLQMNTLTAEDTAVYYCARETWNHLFDYWGLGTLVTVSSAAAHHHHHHHH

SEQ ID NO: 9 - protein, 8 aa, Artificial sequence
  Synthetic construct
  MISC_FEATURE: GS linker
GGGGTGGS

SEQ ID NO: 10 - protein, 16 aa, Artificial sequence
  Synthetic construct
  MISC_FEATURE: 3C linker
GGGSLEVLFQGPGSGS

SEQ ID NO: 11 - protein, 8 aa, Artificial sequence
  Synthetic construct
  MISC_FEATURE: 3C linker
LEVLFQGP

SEQ ID NO: 12 - protein, 4 aa, Artificial sequence
  Synthetic construct
GGGS

SEQ ID NO: 13 - protein, 4 aa, Artificial sequence
  Synthetic construct
SGGG

SEQ ID NO: 14 - protein, 5 aa, Artificial sequence
  Synthetic construct
GGGGS

SEQ ID NO: 15 - protein, 5 aa, Artificial sequence
  Synthetic construct
SGGGG

SEQ ID NO: 16 - protein, 6 aa, Artificial sequence
  Synthetic construct
GGGGGS

SEQ ID NO: 17 - protein, 6 aa, Artificial sequence
  Synthetic construct
SGGGGG

SEQ ID NO: 18 - protein, 7 aa, Artificial sequence
  Synthetic construct
GGGGGGS

SEQ ID NO: 19 - protein, 7 aa, Artificial sequence
  Synthetic construct
SGGGGGG

SEQ ID NO: 20 - protein, 250 aa, Artificial sequence
  Synthetic construct
  MISC_FEATURE: Bases 1-250 may be present or absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 21 - protein, 250 aa, Artificial sequence
  Synthetic construct
  MISC_FEATURE: Bases 1-250 may be present or absent
SGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGG

SEQ ID NO: 22 - protein, 25 aa, Artificial sequence
  Synthetic construct
  MISC_FEATURE: Bases 1-20 may be present or absent
  MISC_FEATURE (1)..(22): X is any amino acid that can be inserted into the sequence and not result in a polypeptide comprising the sequence GSG
GGGXXGGGXXGGGXXGGGXXGGGGS

SEQ ID NO: 23 - protein, 25 aa, Artificial sequence
  Synthetic construct
  MISC_FEATURE: Bases 6-25 may be present or absent
  MISC_FEATURE (1)..(25): X is any amino acid that can be inserted into the sequence and not result in a polypeptide comprising the sequence GSG
GGGGSXGGGSXGGGSXGGGSXGGGS

SEQ ID NO: 24 - protein, 25 aa, Artificial sequence
  Synthetic construct
  MISC_FEATURE: Bases 1-20 may be present or absent
  MISC_FEATURE (4)..(4): Xaa is proline
  MISC_FEATURE (5)..(5): Xaa is serine
  MISC_FEATURE (9)..(9): Xaa is proline
  MISC_FEATURE (10)..(10): Xaa is serine
  MISC_FEATURE (14)..(14): Xaa is proline
  MISC_FEATURE (15)..(15): Xaa is serine
  MISC_FEATURE (19)..(19): Xaa is proline
  MISC_FEATURE (20)..(20): Xaa is serine
GGGXXGGGXXGGGXXGGGXXGGGGS

SEQ ID NO: 25 - protein, 25 aa, Artificial sequence
  Synthetic construct
  MISC_FEATURE: Bases 1-20 may be present or absent
  MISC_FEATURE (4)..(4): Xaa is glycine
  MISC_FEATURE (5)..(5): Xaa is glutamine
  MISC_FEATURE (9)..(9): Xaa is glycine
  MISC_FEATURE (10)..(10): Xaa is glutamine
  MISC_FEATURE (14)..(14): Xaa is glycine
  MISC_FEATURE (15)..(15): Xaa is glutamine
  MISC_FEATURE (19)..(19): Xaa is glycine
  MISC_FEATURE (20)..(20): Xaa is glutamine
GGGXXGGGXXGGGXXGGGXXGGGGS

SEQ ID NO: 26 - protein, 25 aa, Artificial sequence
  Synthetic construct
  MISC_FEATURE: Bases 1-20 may be present or absent
  MISC_FEATURE (4)..(4): Xaa is glycine
  MISC_FEATURE (5)..(5): Xaa is alanine
  MISC_FEATURE (9)..(9): Xaa is glycine
  MISC_FEATURE (10)..(10): Xaa is alanine
  MISC_FEATURE (14)..(14): Xaa is glycine
  MISC_FEATURE (15)..(15): Xaa is alanine
  MISC_FEATURE (19)..(19): Xaa is glycine
  MISC_FEATURE (20)..(20): Xaa is alanine
GGGXXGGGXXGGGXXGGGXXGGGGS

SEQ ID NO: 27 - protein, 25 aa, Artificial sequence
  Synthetic construct
  MISC_FEATURE: Bases 6-25 may be present or absent
  MISC_FEATURE (6)..(21): Xaa is proline
GGGGSXGGGSXGGGSXGGGSXGGGS

SEQ ID NO: 28 - protein, 15 aa, Artificial sequence
  Synthetic construct
GGGGAGGGGAGGGGS

SEQ ID NO: 29 - protein, 15 aa, Artificial sequence
  Synthetic construct
GGGGQGGGGQGGGGS

SEQ ID NO: 30 - protein, 15 aa, Artificial sequence
  Synthetic construct
GGGPSGGGPSGGGGS

SEQ ID NO: 31 - protein, 15 aa, Artificial sequence
  Synthetic construct
GGGGSPGGGSPGGGS

SEQ ID NO: 32 - protein, 8 aa, Artificial sequence
  Synthetic construct
  MISC_FEATURE: Ovalbumin peptide 257-264
SIINFEKL

SEQ ID NO: 33 - protein, 17 aa, Artificial sequence
  Synthetic construct
  MISC_FEATURE: Ovalbumin peptide 323-239
ISQAVHAAHAEINEAGR

SEQ ID NO: 34 - protein, 11 aa, Artificial sequence
  Synthetic construct
AAAHHHHHHHH